(19) United States
(12) Patent Application Publication
(10) Pub. No.: US 2008/0139564 A1
(43) Pub. Date: Jun. 12, 2008

(54) SUBSTITUTED PHENYL PROPYL AMINES AS HISTAMINE H3 RECEPTOR AND SEROTONIN TRANSPORTER MODULATORS

(76) Inventors: John M. Keith, San Diego, CA (US); Jennifer M.B. Miller, Kenmore, WA (US); Emily M. Stocking, Encinitas, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/939,881

(22) Filed: Nov. 14, 2007

Related U.S. Application Data
(60) Provisional application No. 60/866,112, filed on Nov. 16, 2006.

(51) Int. Cl. A61K 31/5375; A61P 25/28; C07D 211/06; A61K 31/445; C07D 295/027; A61K 31/496

(52) U.S. Cl. 514/237.8; 546/217; 514/327; 544/383; 514/252.12; 544/162

(57) ABSTRACT

Certain substituted phenyl propyl amine compounds are histamine H₃ receptor and/or serotonin transporter modulators useful in the treatment of histamine H₃ receptor- and/or serotonin-mediated diseases.

### CROSS-REFERENCE TO RELATED APPLICATIONS

This application claims the benefit of U.S. Provisional Application 60/866,112, filed Nov. 16, 2006, which is hereby incorporated by reference in its entirety.

### FIELD OF THE INVENTION

The present invention relates to certain substituted phenyl propyl amine compounds, pharmaceutical compositions containing them, and methods of using them for the treatment of disease states, disorders, and conditions mediated by histamine H₃ receptor and/or serotonin transporter activity.

### BACKGROUND OF THE INVENTION

The histamine H₃ receptor is primarily expressed in the mammalian central nervous system (CNS), with some minimal expression in peripheral tissues such as vascular smooth muscle. Several indications for histamine H₃ antagonists and inverse agonists have been proposed based on animal pharmacology and other experiments with known histamine H₃ antagonists (e.g. thioperamide). (See: Krause et al. and Phillips et al. in “The Histamine H₃ Receptor—A Target for New Drugs”, Leurs, R. and Timmerman, H., (Eds.), Elsevier, 1998, pp. 175-196 and 197-222; Morisset, S. et al., Nature 2000, 408, 860-864; Bonaventure et al., Biochem. Pharm. 2007, 73, 1084-1096; Letavic et al., Prog. Med. Chem. 2006, 44, 181-206.) These include conditions such as cognitive disorders, sleep disorders, psychiatric disorders, and other disorders.

Compounds that possess histamine H₃ receptor activity and serotonin transporter (SERT) activity may be useful in the treatment of SERT-mediated disorders such as substance abuse disorders and sexual dysfunction (including premature ejaculation), and particularly beneficial in the treatment of depression. Activation of the H₃ receptor on neurons by histamine or an agonist decreases the release of several neurotransmitters including noradrenaline and serotonin, key neurotransmitters involved in depression (Hill, S. J. et al. Pharmacol. Rev. 1997, 49(3), 253-278). Although H₃ receptor antagonists alone may not be capable of increasing serotonin levels in vivo to those required for antidepressant effects, concomitant blockade of the SERT will simultaneously decrease the neuronal reuptake of these neurotransmitter molecules, leading to enhanced concentrations of serotonin in the synaptic cleft and an enhanced therapeutic effect and a potentially reduced side effect profile as compared to a compound with SERT activity alone.

Histamine H₃ antagonists have been shown to have pharmacological activity relevant to several key symptoms of depression, including sleep disorders (e.g. sleep disturbances, fatigue, and lethargy) and cognitive difficulties (e.g. memory and concentration impairment), as described above. Therefore, a combined H₃/SERT modulating compound would provide symptomatic relief for the sleep disorders, fatigue, and cognitive problems during the first weeks of treatment, before the mood-elevating effect of the SERT modulation is noticed.

Compounds that have H₃ receptor activity and SERT activity have been disclosed in U.S. Patent Appl. Publ. US 2006/0194837 (Aug. 31, 2006), U.S. Patent Appl. Publ. US 2006/0293316, and U.S. Patent Appl. Publ. US 2006/0287292, which are each hereby incorporated by reference. Phenoxyphenyl diamines were described by Stocking et al. (Bioorg. Med. Chem. Lett. 2007, 17, 3130-3135). Propanolamines were described as calcium channel blockers in European Pat. Appl. EP 0576766.

However, there remains a need for potent histamine H₃ receptor and/or serotonin transporter modulators with desirable pharmaceutical properties.

### SUMMARY OF THE INVENTION

Certain substituted phenyl propyl amine derivatives have now been found to have histamine H₃ receptor and/or serotonin transporter modulating activity. Thus, the invention is directed to the general and preferred embodiments defined, respectively, by the independent and dependent claims appended hereto, which are incorporated by reference herein.

In one general aspect the invention relates to a compound of the following Formula (I):

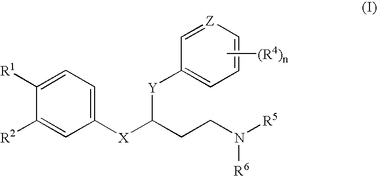

wherein:
one of X and Y is O, S, NH, or CH₂, and the other is a bond;
Z is CH or N, with the proviso that Z is N only when Y is O;
one of R¹ and R² is -Q and the other is —H;
   -Q is —OCH(R^{a})(CH₂)₂NR^{b}R^{c}, —C≡C(CH₂)₂NR^{b}R^{c}, —(CH₂)₄NR^{b}R^{c}, —CH₂NR^{b}R^{c}, or —C(O)NR^{b}R^{c};
   wherein R^{a} is —H or is taken together with R^{b} to form ethylene; and
   R^{b} and R^{c} are —H or —C₁₋₆alkyl, or R^{b} and R^{c} taken together with their nitrogen of attachment form a heterocycloalkyl ring, unsubstituted or substituted with C₁₋₆alkyl, C₃₋₆cycloalkyl, fluoro, or —CN;
each R⁴ substituent is independently selected from the group consisting of halo, —C₁₋₆alkyl, —CHF₂, —CF₃, —OH, —OC₁₋₆alkyl, —OCHF₂, —OCF₃, —CN, —N(R^{k})R^{l}, —NO₂, —SC₁₋₆alkyl, —SCF₃, and —S(O)₀₋₂—C₁₋₆alkyl; or, alternatively, only when Y is O, two adjacent R⁴ substituents taken together with the phenyl to which they are attached form indol-5-yl;
   wherein R^{k} and R^{l} are each independently —H or —C₁₋₆alkyl;
n is 0, 1, 2, or 3;
R⁵ is —H or —C₁₋₄alkyl; and R⁶ is —C₁₋₄alkyl, or, alternatively, R⁵ and R⁶ taken together with their nitrogen of attachment form a heterocycloalkyl ring;
   or a pharmaceutically acceptable salt, pharmaceutically acceptable prodrug, or pharmaceutically active metabolite of such compound.

In a further general aspect, the invention relates to pharmaceutical compositions each comprising: (a) an effective amount of a compound of Formula (I), or a pharmaceutically acceptable salt, pharmaceutically acceptable prodrug, or pharmaceutically active metabolite thereof; and (b) a pharmaceutically acceptable excipient.

In another general aspect, the invention is directed to a method of treating a subject suffering from or diagnosed with a disease, disorder, or medical condition mediated by histamine H₃ receptor and/or serotonin transporter activity, comprising administering to the subject in need of such treatment an effective amount of a compound of Formula (I), or a pharmaceutically acceptable salt, pharmaceutically acceptable prodrug, or pharmaceutically active metabolite thereof.

In certain preferred embodiments of the inventive method, the disease, disorder, or medical condition is selected from: cognitive disorders, sleep disorders, psychiatric disorders, and other disorders.

Additional embodiments, features, and advantages of the invention will be apparent from the following detailed description and through practice of the invention.

### DETAILED DESCRIPTION OF INVENTION AND ITS PREFERRED EMBODIMENTS

The invention may be more fully appreciated by reference to the following description, including the following glossary of terms and the concluding examples. For the sake of brevity, the disclosures of the publications, including patents, cited in this specification are herein incorporated by reference.

As used herein, the terms “including”, “containing” and “comprising” are used herein in their open, non-limiting sense.

The term “alkyl” refers to a straight- or branched-chain alkyl group having from 1 to 12 carbon atoms in the chain. Examples of alkyl groups include methyl (Me, which also may be structurally depicted by a/symbol), ethyl (Et), n-propyl (Pr), isopropyl (iPr), butyl (Bu), isobutyl (iBu), sec-butyl (sBu), tert-butyl (tBu), pentyl, isopentyl, tert-pentyl, hexyl, isohexyl, and groups that in light of the ordinary skill in the art and the teachings provided herein would be considered equivalent to any one of the foregoing examples.

The term “alkylene” refers to a straight- or branched-chain alkyl group having from 1 to 12 carbon atoms in the chain, where two hydrogen atoms are removed to for a diradical. Examples of alkylene groups include methylene (—CH₂—), ethylene, n-propylene, isopropylene, butylene, and groups that in light of the ordinary skill in the art and the teachings provided herein would be considered equivalent to any one of the foregoing examples.

The term “alkenyl” refers to a straight- or branched-chain alkenyl group having from 2 to 12 carbon atoms in the chain. (The double bond of the alkenyl group is formed by two sp² hybridized carbon atoms.) Illustrative alkenyl groups include prop-2-enyl, but-2-enyl, but-3-enyl, 2-methylprop-2-enyl, hex-2-enyl, and groups that in light of the ordinary skill in the art and the teachings provided herein would be considered equivalent to any one of the foregoing examples.

The term “alkynyl” refers to a straight- or branched-chain alkynyl group having from 2 to 12 carbon atoms in the chain. (The triple bond of the alkynyl group is formed by two sp hybridized carbon atoms.) Illustrative alkynyl groups include ethynyl, propynyl, butynyl, hexynyl, and groups that in light of the ordinary skill in the art and the teachings provided herein would be considered equivalent to any one of the foregoing examples.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

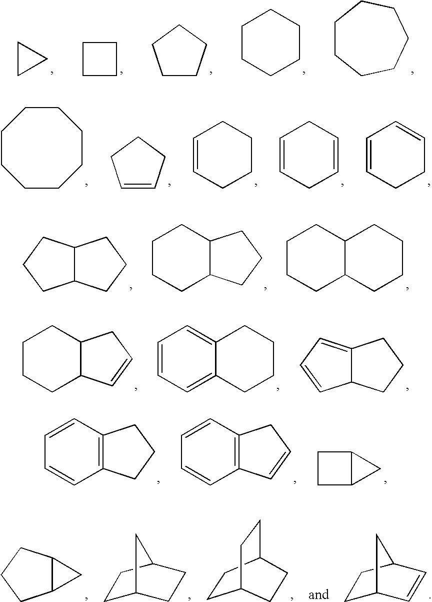

A “heterocycloalkyl” refers to a monocyclic ring structure that is saturated or partially saturated and has from 4 to 7 ring atoms per ring structure selected from carbon atoms and up to two heteroatoms selected from nitrogen, oxygen, and sulfur. The ring structure may optionally contain up to two oxo groups on sulfur ring members. Illustrative entities, in the form of properly bonded moieties, include:

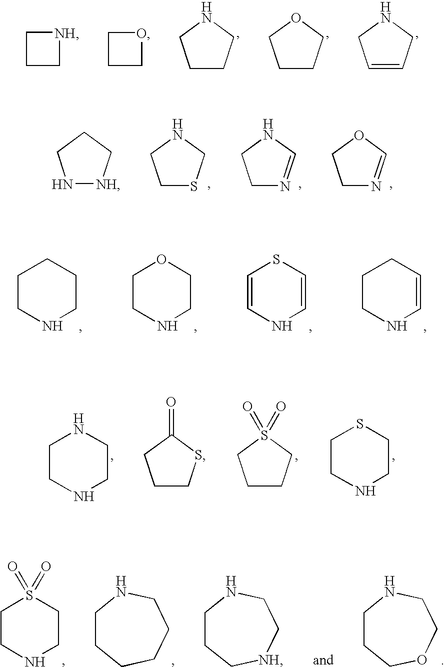

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

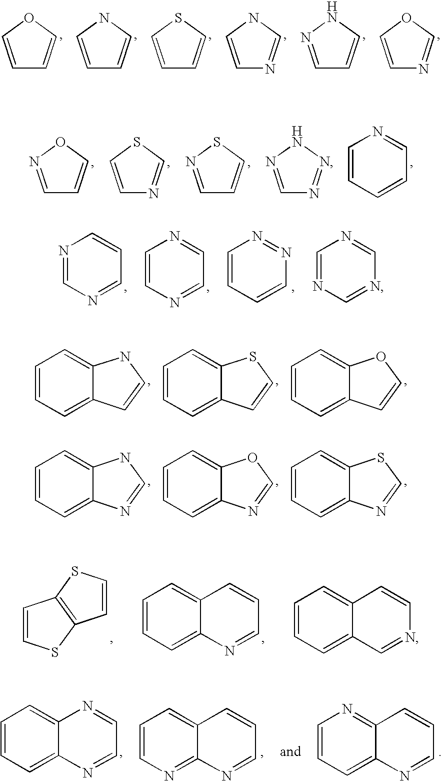

Those skilled in the art will recognize that the species of heteroaryl, cycloalkyl, and heterocycloalkyl groups listed or illustrated above are not exhaustive, and that additional species within the scope of these defined terms may also be selected.

The term “halogen” represents chlorine, fluorine, bromine or iodine. The term “halo” represents chloro, fluoro, bromo or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to embrace hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula.

In preferred embodiments of Formula (I), X is O, S, NH, or CH₂ and Y is a bond. In other embodiments, Y is O, S, NH, or CH₂ and X is a bond. In further preferred embodiments, X is a bond and Y is O.

In preferred embodiments, Z is CH.

In preferred embodiments, R² is -Q and R¹ is —H. In other embodiments, R¹ is -Q and R² is —H.

In preferred embodiments, -Q is —O(CH₂)₃NR^{b}R^{c}. In other preferred embodiments, -Q is —C≡C(CH₂)₂NR^{b}R^{c} or —(CH₂)₄NR^{b}R^{c}. In still other preferred embodiments, -Q is —CH₂NR^{b}R^{c} or —C(O)NR^{b}R^{c}. In further preferred embodiments, R¹ is -Q, R² is —H, and -Q is —O(CH₂)₃NR^{b}R^{c}.

In preferred embodiments, R^{a} is —H.

In preferred embodiments, R^{b} and R^{c} are each independently —H, methyl, ethyl, propyl, isopropyl, butyl, isobutyl, tert-butyl, pentyl, or hexyl. In further preferred embodiments, R^{b} and R^{c} taken together with their nitrogen of attachment form a 5- to 7-membered heterocycloalkyl ring, unsubstituted or substituted with methyl, ethyl, isopropyl, butyl, isobutyl, sec-butyl, isopentyl, cyclopropyl, cyclobutyl, cyclopentyl, cyclohexyl, fluoro, or cyano. In still further preferred embodiments, R^{b} and R^{c} taken together with their nitrogen of attachment form diazepine, piperidine, piperazine, morpholine, or thiomorpholine, each unsubstituted or substituted with methyl, ethyl, isopropyl, butyl, isobutyl, sec-butyl, isopentyl, cyclopropyl, cyclobutyl, cyclopentyl, cyclohexyl, fluoro, or cyano. In still further preferred embodiments, R^{b} and R^{c} taken together with their nitrogen of attachment form piperidin-1-yl, 4-fluoro-piperidin-1-yl, 4-cyano-piperidin-1-yl, 4-isopropyl-piperazin-1-yl, morpholinyl, 3-methyl-morpholin-1-yl, thiomorpholinyl, 4-butyl-piperazin-1-yl, 4-cyclopropylpiperazin-1-yl, 4-sec-butyl-piperazin-1-yl, 4-(1-ethyl-propyl)-piperazin-1-yl, 4-cyclopentyl-piperazin-1-yl, or 4-cyclohexyl-piperazin-1-yl.

In preferred embodiments, each R⁴ substituent is independently selected from the group consisting of chloro, bromo, methyl, —CF₃, methoxy, —NO₂, and methanesulfanyl.

In preferred embodiments, n is 0, 1, or 2. In further preferred embodiments, n is 1 or 2.

In preferred embodiments, R⁵ and R⁶ are each independently —H or methyl. In preferred embodiments, R⁵ and R⁶ are both methyl. In other preferred embodiments, R⁵ and R⁶ taken together with their nitrogen of attachment form azetidinyl, pyrrolidinyl, or piperidinyl.

In some embodiments, compounds of Formula (I) are selected from compounds of Formula (II):

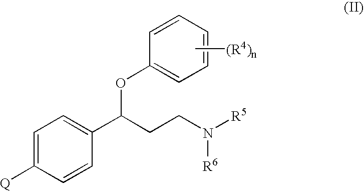

wherein:
-Q is —OCH(R^{a})(CH₂)₂NR^{b}R^{c}, —C≡C(CH₂)₂NR^{b}R^{c}, —(CH₂)₄NR^{b}R^{c}, —CH₂NR^{b}R^{c}, or —C(O)NR^{b}R^{c};
   wherein R^{a} is —H or is taken together with R^{b} to form ethylene; and
   R^{b} and R^{c} are —H or —C₁₋₆alkyl, or R^{b} and R^{c} taken together with their nitrogen of attachment form a heterocycloalkyl ring, unsubstituted or substituted with C₁₋₆alkyl, C₃₋₆cycloalkyl, fluoro, or —CN;
each R⁴ substituent is independently selected from the group consisting of halo, —C₁₋₆alkyl, —CHF₂, —CF₃, —OH, —OC₁₋₆alkyl, —OCHF₂, —OCF₃, —CN, —N(R^{k})R^{l}, —NO₂, —SC₁₋₆alkyl, —SCF₃, and —S(O)₀₋₂—C₁₋₆alkyl; or, alternatively, two adjacent R⁴ substituents taken together with the phenyl to which they are attached form indol-5-yl;
   wherein R^{k} and R^{l} are each independently —H or —C₁₋₆alkyl;
n is 0, 1, 2, or 3;
R⁵ is —H or —C₁₋₄alkyl; and R⁶ is —C₁₋₄alkyl, or, alternatively, R⁵ and R⁶ taken together with their nitrogen of attachment form a heterocycloalkyl ring;
   or a pharmaceutically acceptable salt, pharmaceutically acceptable prodrug, or pharmaceutically active metabolite of such compound.

In other embodiments of Formula (II), -Q is —OCH(R^{a})(CH₂)₂NR^{b}R^{c}.

In certain preferred embodiments, the compound of Formula (I) is selected from the group consisting of:

| Ex. | Compound Name |
|---|---|
| 1 | Dimethyl-{3-phenoxy-3-[4-(3-piperidin-1-yl-propoxy)-phenyl]-propyl}- |
| | amine; |
| 2 | Dimethyl-[3-[4-(3-piperidin-1-yl-propoxy)-phenyl]-3-(4-trifluoromethyl- |
| | phenoxy)-propyl]-amine; |
| 3 | Dimethyl-{3-phenoxy-3-[3-(3-piperidin-1-yl-propoxy)-phenyl]-propyl}- |
| | amine; |
| 4 | Dimethyl-{3-(4-methylsulfanyl-phenoxy)-3-[4-(3-piperidin-1-yl-propoxy)- |
| | phenyl]-propyl}-amine; |
| 5 | 4-{3-Dimethylamino-1-[4-(3-piperidin-1-yl-propoxy)-phenyl]-propoxy}- |
| | benzonitrile; |
| 6 | Dimethyl-{3-[4-(3-piperidin-1-yl-propoxy)-phenyl]-3-p-tolyloxy-propyl}- |
| | amine; |
| 7 | {3-(4-Methoxy-phenoxy)-3-[4-(3-piperidin-1-yl-propoxy)-phenyl]-propyl}- |
| | dimethyl-amine; |
| 8 | {3-(4-Chloro-phenoxy)-3-[4-(3-piperidin-1-yl-propoxy)-phenyl]-propyl}- |
| | dimethyl-amine; |
| 9 | {3-(3,4-Dichloro-phenoxy)-3-[4-(3-piperidin-1-yl-propoxy)-phenyl]- |
| | propyl}-dimethyl-amine; |
| 10 | {3-(4-Fluoro-phenoxy)-3-[4-(3-piperidin-1-yl-propoxy)-phenyl]-propyl}- |
| | dimethyl-amine; |
| 11 | {3-(4-Bromo-phenoxy)-3-[4-(3-piperidin-1-yl-propoxy)-phenyl]-propyl}- |
| | dimethyl-amine; |
| 12 | Dimethyl-{3-(4-nitro-phenoxy)-3-[4-(3-piperidin-1-yl-propoxy)-phenyl]- |
| | propyl}-amine; |
| 13 | {3-(3-Methoxy-phenoxy)-3-[4-(3-piperidin-1-yl-propoxy)-phenyl]-propyl}- |
| | dimethyl-amine; |
| 14 | {3-(3-Chloro-phenoxy)-3-[4-(3-piperidin-1-yl-propoxy)-phenyl]-propyl}- |
| | dimethyl-amine; |
| 15 | {3-(3-Bromo-phenoxy)-3-[4-(3-piperidin-1-yl-propoxy)-phenyl]-propyl}- |
| | dimethyl-amine; |
| 16 | {3-(2,4-Dichloro-phenoxy)-3-[4-(3-piperidin-1-yl-propoxy)-phenyl]- |
| | propyl}-dimethyl-amine; |
| 17 | {3-(2-Chloro-phenoxy)-3-[4-(3-piperidin-1-yl-propoxy)-phenyl]-propyl}- |
| | dimethyl-amine; |
| 18 | Dimethyl-[3-[4-(3-piperidin-1-yl-propoxy)-phenyl]-3-(pyridin-3-yloxy)- |
| | propyl]-amine; |
| 19 | {3-(1H-Indol-5-yloxy)-3-[4-(3-piperidin-1-yl-propoxy)-phenyl]-propyl}- |
| | dimethyl-amine; |
| 20 | Dimethyl-[3-[4-(4-piperidin-1-yl-but-1-ynyl)-phenyl]-3-(4-trifluoromethyl- |
| | phenoxy)-propyl]-amine; |
| 21 | Dimethyl-{3-phenoxy-3-[4-(4-piperidin-1-yl-but-1-ynyl)-phenyl]-propyl}- |
| | amine; |
| 22 | (3-{4-[4-(4-Isopropyl-piperazin-1-yl)-but-1-ynyl]-phenyl}-3-phenoxy- |
| | propyl)-dimethyl-amine; |
| 23 | Dimethyl-{3-[4-(4-morpholin-4-yl-but-1-ynyl)-phenyl]-3-phenoxy-propyl}- |
| | amine; |
| 24 | Dimethyl-{3-phenoxy-3-[4-(4-thiomorpholin-4-yl-but-1-ynyl)-phenyl]- |
| | propyl}-amine; |
| 25 | {3-(4-Chloro-phenoxy)-3-[4-(4-piperidin-1-yl-but-1-ynyl)-phenyl]-propyl}- |
| | dimethyl-amine; |
| 26 | {3-(4-Methoxy-phenoxy)-3-[4-(4-thiomorpholin-4-yl-but-1-ynyl)-phenyl]- |
| | propyl}-dimethyl-amine; |
| 27 | Dimethyl-[3-[4-(4-morpholin-4-yl-but-1-ynyl)-phenyl]-3-(4-trifluoromethyl- |
| | phenoxy)-propyl]-amine; |
| 28 | Dimethyl-[3-[4-(4-thiomorpholin-4-yl-but-1-ynyl)-phenyl]-3-(4- |
| | trifluoromethyl-phenoxy)-propyl]-amine; |
| 29 | [3-{4-[4-(4-Isopropyl-piperazin-1-yl)-but-1-ynyl]-phenyl}-3-(4- |
| | trifluoromethyl-phenoxy)-propyl]-dimethyl-amine; |
| 30 | {3-(3,4-Dichloro-phenoxy)-3-[4-(4-piperidin-1-yl-but-1-ynyl)-phenyl]- |
| | propyl}-dimethyl-amine; |
| 31 | Dimethyl-{3-phenoxy-3-[3-(4-piperidin-1-yl-but-1-ynyl)-phenyl]-propyl}- |
| | amine; |
| 32 | Dimethyl-{3-phenoxy-3-[4-(4-piperidin-1-yl-butyl)-phenyl]-propyl}-amine; |
| 33 | Dimethyl-[3-[4-(4-piperidin-1-yl-butyl)-phenyl]-3-(4-trifluoromethyl- |
| | phenoxy)-propyl]-amine; |
| 34 | [3-{4-[4-(4-Isopropyl-piperazin-1-yl)-butyl]-phenyl}-3-(4-trifluoromethyl- |
| | phenoxy)-propyl]-dimethyl-amine; |
| 35 | Dimethyl-[3-[4-(4-morpholin-4-yl-butyl)-phenyl]-3-(4-trifluoromethyl- |
| | phenoxy)-propyl]-amine; |
| 36 | {3-[4-(1-Isopropyl-piperidin-4-yloxy)-phenyl]-3-phenoxy-propyl}-dimethyl- |
| | amine; |
| 37 | Dimethyl-{3-phenyl-3-[3-(3-piperidin-1-yl-propoxy)-phenoxy]-propyl}- |
| | amine; |
| 38 | [4-(3-Dimethylamino-1-phenoxy-propyl)-phenyl]-(4-isopropyl-piperazin- |
| | 1-yl)-methanone; |
| 39 | {4-[3-Dimethylamino-1-(4-trifluoromethyl-phenoxy)-propyl]-phenyl}-(4- |
| | isopropyl-piperazin-1-yl)-methanone; |
| 40 | {4-[1-(3,4-Dichloro-phenoxy)-3-dimethylamino-propyl]-phenyl}-(4- |
| | isopropyl-piperazin-1-yl)-methanone; |
| 41 | Dimethyl-{3-phenyl-3-[4-(3-piperidin-1-yl-propoxy)-phenoxy]-propyl}- |
| | amine; |
| 42 | Dimethyl-[3-[4-(3-piperidin-1-yl-propoxy)-phenoxy]-3-(4-trifluoromethyl- |
| | phenyl)-propyl]-amine; |
| 43 | {3-(4-Chloro-phenyl)-3-[4-(3-piperidin-1-yl-propoxy)-phenoxy]-propyl}- |
| | dimethyl-amine; |
| 44 | {3-[4-(1-Isopropyl-piperidin-4-yloxy)-phenoxy]-3-phenyl-propyl}-dimethyl- |
| | amine; |
| 45 | [3-[4-(1-Isopropyl-piperidin-4-yloxy)-phenoxy]-3-(4-trifluoromethyl- |
| | phenyl)-propyl]-dimethyl-amine; |
| 46 | {4-[3-Dimethylamino-1-(4-trifluoromethyl-phenyl)-propoxy]-phenyl}-(4- |
| | isopropyl-piperazin-1-yl)-methanone; |
| 47 | {3-(3,4-Dichloro-phenoxy)-3-[4-(4-isopropyl-piperazin-1-ylmethyl)- |
| | phenyl]-propyl}-dimethyl-amine; |
| 48 | (4-Butyl-piperazin-1-yl)-{4-[3-dimethylamino-1-(4-trifluoromethyl- |
| | phenoxy)-propyl]-phenyl}-methanone; |
| 49 | [3-[4-(4-Butyl-piperazin-1-ylmethyl)-phenyl]-3-(4-trifluoromethyl- |
| | phenoxy)-propyl]-dimethyl-amine; |
| 50 | (4-Cyclopentyl-piperazin-1-yl)-{4-[3-dimethylamino-1-(4-trifluoromethyl- |
| | phenoxy)-propyl]-phenyl}-methanone; |
| 51 | [3-[4-(4-Cyclopentyl-piperazin-1-ylmethyl)-phenyl]-3-(4-trifluoromethyl- |
| | phenoxy)-propyl]-dimethyl-amine; |
| 52 | (4-sec-Butyl-piperazin-1-yl)-{4-[3-dimethylamino-1-(4-trifluoromethyl- |
| | phenoxy)-propyl]-phenyl}-methanone; |
| 53 | {4-[3-Dimethylamino-1-(4-trifluoromethyl-phenoxy)-propyl]-phenyl}-[4-(1- |
| | ethyl-propyl)-piperazin-1-yl]-methanone; |
| 54 | [3-{4-[4-(1-Ethyl-propyl)-piperazin-1-ylmethyl]-phenyl}-3-(4- |
| | trifluoromethyl-phenoxy)-propyl]-dimethyl-amine; |
| 55 | [3-[4-(4-Isopropyl-piperazin-1-ylmethyl)-phenyl]-3-(4-trifluoromethyl- |
| | phenoxy)-propyl]-dimethyl-amine; |
| 56 | {3-[4-(4-Isopropyl-piperazin-1-ylmethyl)-phenyl]-3-phenoxy-propyl}- |
| | dimethyl-amine; |
| 57 | Dimethyl-{3-phenyl-3-[4-(4-piperidin-1-yl-but-1-ynyl)-phenoxy]-propyl}- |
| | amine; |
| 58 | {3-(4-Chloro-phenyl)-3-[4-(4-thiomorpholin-4-yl-but-1-ynyl)-phenoxy]- |
| | propyl}-dimethyl-amine; |
| 59 | {3-(4-Chloro-phenyl)-3-[4-(4-morpholin-4-yl-but-1-ynyl)-phenoxy]- |
| | propyl}-dimethyl-amine; |
| 60 | Dimethyl-[3-[4-(4-piperidin-1-yl-but-1-ynyl)-phenoxy]-3-(4- |
| | trifluoromethyl-phenyl)-propyl]-amine; |
| 61 | Dimethyl-{3-[4-(4-morpholin-4-yl-but-1-ynyl)-phenoxy]-3-phenyl-propyl}- |
| | amine; |
| 62 | [3-{4-[4-(4-Isopropyl-piperazin-1-yl)-but-1-ynyl]-phenoxy}-3-(4- |
| | trifluoromethyl-phenyl)-propyl]-dimethyl-amine; |
| 63 | Dimethyl-{3-phenyl-3-[4-(4-thiomorpholin-4-yl-but-1-ynyl)-phenoxy]- |
| | propyl}-amine; |
| 64 | Dimethyl-[3-[4-(4-morpholin-4-yl-but-1-ynyl)-phenoxy]-3-(4- |
| | trifluoromethyl-phenyl)-propyl]-amine; |
| 65 | Dimethyl-{4-phenyl-3-[4-(3-piperidin-1-yl-propoxy)-phenyl]-butyl}-amine; |
| 66 | N³,N³-Dimethyl-N¹-phenyl-1-[4-(3-piperidin-1-yl-propoxy)-phenyl]- |
| | propane-1,3-diamine; |
| 67 | Dimethyl-{3-phenylsulfanyl-3-[4-(3-piperidin-1-yl-propoxy)-phenyl]- |
| | propyl}-amine; |
| 68 | 3-Phenoxy-3-[4-(3-piperidin-1-yl-propoxy)-phenyl]-propylamine; |
| 69 | 3-[4-(3-Piperidin-1-yl-propoxy)-phenyl]-3-(4-trifluoromethyl-phenoxy)- |
| | propylamine; |
| 70 | Methyl-[3-[4-(3-piperidin-1-yl-propoxy)-phenyl]-3-(4-trifluoromethyl- |
| | phenoxy)-propyl]-amine; |
| 71 | {3-(3,4-Dichloro-phenoxy)-3-[4-(3-piperidin-1-yl-propoxy)-phenyl]- |
| | propyl}-methyl-amine; |
| 72 | Methyl-{3-phenoxy-3-[4-(3-piperidin-1-yl-propoxy)-phenyl]-propyl}- |
| | amine; |
| 73 | 1-{3-[4-(1-Phenoxy-3-pyrrolidin-1-yl-propyl)-phenoxy]-propyl}-piperidine; |
| 74 | (−)-{3-(3,4-Dichloro-phenoxy)-3-[4-(3-piperidin-1-yl-propoxy)-phenyl]- |
| | propyl}-dimethyl-amine; |
| 75 | (+)-{3-(3,4-Dichloro-phenoxy)-3-[4-(3-piperidin-1-yl-propoxy)-phenyl]- |
| | propyl}-dimethyl-amine; |
| 76 | Dimethyl-[3-[4-(4-thiomorpholin-4-yl-but-1-ynyl)-phenoxy]-3-(4- |
| | trifluoromethyl-phenyl)-propyl]-amine; |
| 77 | {3-(4-Fluoro-phenyl)-3-[4-(4-piperidin-1-yl-but-1-ynyl)-phenoxy]-propyl}- |
| | dimethyl-amine; |
| 78 | (4-Cyclohexyl-piperazin-1-yl)-{4-[3-dimethylamino-1-(4-trifluoromethyl- |
| | phenoxy)-propyl]-phenyl}-methanone; |
| 79 | {3-[4-(4-Cyclohexyl-piperazin-1-ylmethyl)-phenyl]-3-phenoxy-propyl}- |
| | dimethyl-amine; |
| 80 | (3-{4-[3-(4-Fluoro-piperidin-1-yl)-propoxy]-phenyl}-3-phenoxy-propyl)- |
| | dimethyl-amine; |
| 81 | 1-{3-[4-(3-Dimethylamino-1-phenoxy-propyl)-phenoxy]-propyl}- |
| | piperidine-4-carbonitrile; |
| 82 | Dimethyl-(3-{4-[3-(2-methyl-morpholin-4-yl)-propoxy]-phenyl}-3- |
| | phenoxy-propyl)-amine; |
| 83 | (4-Cyclopropyl-[1,4]diazepan-1-yl)-{4-[3-dimethylamino-1-(4- |
| | trifluoromethyl-phenoxy)-propyl]-phenyl}-methanone; and |
| 84 | 1-{3-[4-(3-Azetidin-1-yl-1-phenoxy-propyl)-phenoxy]-propyl}-piperidine; |

and pharmaceutically acceptable salts thereof.

The invention includes also pharmaceutically acceptable salts of the compounds represented by Formula (I), preferably of those described above and of the specific compounds exemplified herein, and methods of treatment using such salts.

A “pharmaceutically acceptable salt” is intended to mean a salt of a free acid or base of a compound represented by Formula (I) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

If the compound of Formula (I) is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to pharmaceutically acceptable prodrugs of the compounds of Formula (I), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of a compound of Formula (I). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone.

Additional types of prodrugs may be produced, for instance, by derivatizing free carboxyl groups of structures of Formula (I) as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Adv. Drug Delivery Rev. 1996, 19, 115. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in J. Med. Chem. 1996, 39, 10. Free amines can also be derivatized as amides, sulfonamides or phosphonamides. All of these prodrug moieties may incorporate groups including ether, amine, and carboxylic acid functionalities.

The present invention also relates to pharmaceutically active metabolites of compounds of Formula (I), and uses of such metabolites in the methods of the invention. A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini, et al. J. Med. Chem. 1997, 40, 2011-2016; Shan, et al. J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

The compounds of Formula (I) and their pharmaceutically acceptable salts, pharmaceutically acceptable prodrugs, and pharmaceutically active metabolites of the present invention are useful as modulators of the histamine H₃ receptor and/or the serotonin transporter in the methods of the invention. Accordingly, the invention relates to methods of using the compounds of the invention to treat subjects diagnosed with or suffering from a disease, disorder, or condition mediated by histamine H₃ receptor and/or serotonin transporter activity, such as those described herein.

The term “treat” or “treating” as used herein is intended to refer to administration of a compound or composition of the invention to a subject for the purpose of effecting a therapeutic or prophylactic benefit through modulation of histamine H₃ receptor and/or serotonin transporter activity. Treating includes reversing, ameliorating, alleviating, inhibiting the progress of, lessening the severity of, or preventing a disease, disorder, or condition, or one or more symptoms of such disease, disorder or condition mediated through modulation of histamine H₃ receptor and/or serotonin transporter activity. The term “subject” refers to a mammalian patient in need of such treatment, such as a human. “Modulators” include both inhibitors and activators, where “inhibitors” refer to compounds that decrease, prevent, inactivate, desensitize or down-regulate histamine H₃ receptor and/or serotonin transporter expression or activity, and “activators” are compounds that increase, activate, facilitate, sensitize, or up-regulate histamine H₃ receptor and/or serotonin transporter expression or activity.

Exemplary medical conditions, diseases, and disorders include cognitive disorders, sleep disorders, psychiatric disorders, and other disorders. Symptoms or disease states are intended to be included within the scope of “medical conditions, disorders, or diseases.”

Cognitive disorders include, for example, dementia, Alzheimer's disease (Panula, P. et al. Soc. Neurosci. Abstr. 1995, 21, 1977), cognitive dysfunction, mild cognitive impairment (pre-dementia), attention deficit hyperactivity disorders (ADHD), attention-deficit disorders, and learning and memory disorders (Barnes, J. C. et al. Soc. Neurosci. Abstr. 1993, 19, 1813). Learning and memory disorders include, for example, learning impairment, memory impairment, age-related cognitive decline, and memory loss. H₃ antagonists have been shown to improve memory in a variety of memory tests, including the elevated plus maze in mice (Miyazaki, S. et al. Life Sci. 1995, 57(23), 2137-2144), a two-trial place recognition task (Orsetti, M. et al. Behav. Brain Res. 2001, 124(2), 235-242), the passive avoidance test in mice (Miyazaki, S. et al. Meth. Find. Exp. Clin. Pharmacol. 1995, 17(10), 653-658) and the radial maze in rats (Chen, Z. Acta Pharmacol. Sin. 2000, 21(10), 905-910). Also, in the spontaneously hypertensive rat, an animal model for the learning impairments in attention-deficit disorders, H₃ antagonists were shown to improve memory (Fox, G. B. et al. Behav. Brain Res. 2002, 131(1-2), 151-161).

Sleep disorders include, for example, insomnia, disturbed sleep, narcolepsy (with or without associated cataplexy), cataplexy, disorders of sleep/wake homeostasis, idiopathic somnolence, excessive daytime sleepiness (EDS), circadian rhythm disorders, fatigue, lethargy, jet lag (phase delay), and REM-behavioral disorder. Fatigue and/or sleep impairment may be caused by or associated with various sources, such as, for example, sleep apnea, perimenopausal hormonal shifts, Parkinson's disease, multiple sclerosis (MS), depression, chemotherapy, or shift work schedules.

Psychiatric disorders include, for example, schizophrenia (Schlicker, E. and Marr, I. Naunyn-Schmiedeberg's Arch. Pharmacol. 1996, 353, 290-294), including cognitive deficits and negative symptoms associated with schizophrenia, bipolar disorders, manic disorders, depression (Lamberti, C. et al. Br. J. Pharmacol. 1998, 123(7), 1331-1336; Perez-Garcia, C. et al. Psychopharmacology 1999, 142(2), 215-220) (Also see: Stark, H. et al. Drugs Future 1996, 21(5), 507-520; and Leurs, R. et al. Prog. Drug Res. 1995, 45, 107-165 and references cited therein), including bipolar depression, obsessive-compulsive disorder, and post-traumatic stress disorder.

Other disorders include, for example, motion sickness, vertigo (e.g. vertigo or benign postural vertigo), tinitus, epilepsy (Yokoyama, H. et al. Eur. J. Pharmacol. 1993, 234, 129-133), migraine, neurogenic inflammation, neuropathic pain, Down Syndrome, seizures, eating disorders (Machidori, H. et al. Brain Res. 1992, 590, 180-186), obesity, and substance abuse disorders, sexual dysfunction (including premature ejaculation, movement disorders (e.g. restless legs syndrome), and eye-related disorders (e.g. macular degeneration and retinitis pigmentosis).

Particularly, as modulators of the histamine H₃ receptor and/or the serotonin transporter, the compounds of the present invention are useful in the treatment or prevention of depression, disturbed sleep, narcolepsy, fatigue, lethargy, cognitive impairment, memory impairment, memory loss, learning impairment, attention-deficit disorders, and eating disorders.

In a treatment method according to the invention, an effective amount of a compound according to the invention is administered to a subject suffering from or diagnosed as having such a disease, disorder, or condition. An “effective amount” means an amount or dose sufficient to generally bring about the desired therapeutic or prophylactic benefit in patients in need of such treatment.

Effective amounts or doses of the compounds of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. An exemplary dose is in the range of from about 0.001 to about 200 mg of compound per kg of subject's body weight per day, preferably about 0.01 to 100 mg/kg/day, or about 1 to 35 mg/kg/day, or about 0.1 to 10 mg/kg daily in single or divided dosage units (e.g., BID, TID, QID). For a 70-kg human, an illustrative range for a suitable dosage amount is from about 0.05 to about 7 g/day, or about 0.2 to about 2.5 g/day.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In addition, the compounds of the invention may be used in combination with additional active ingredients in the treatment of the above conditions. In an exemplary embodiment, additional active ingredients are those that are known or discovered to be effective in the treatment of conditions, disorders, or diseases mediated by histamine H₃ receptor and/or serotonin transporter activity or that are active against another target associated with the particular condition, disorder, or disease, such as H₁ receptor antagonists, H₂ receptor antagonists, H₄ receptor antagonists, and neurotransmitter modulators such as serotonin-norepinephrine reuptake inhibitors, selective serotonin reuptake inhibitors (SSRIs), noradrenergic reuptake inhibitors, non-selective serotonin re-uptake inhibitors (NSSRIs), acetylcholinesterase inhibitors (such as tetrahydroaminoacridine, donepezil, rivastigmine, or galantamine), or modafinil. The combination may serve to increase efficacy (e.g., by including in the combination a compound potentiating the potency or effectiveness of a compound according to the invention), decrease one or more side effects, or decrease the required dose of the compound according to the invention.

More particularly, compounds of the invention in combination with modafinil are useful for the treatment of narcolepsy, excessive daytime sleepiness (EDS), Alzheimer's disease, depression, attention-deficit disorders, MS-related fatigue, post-anesthesia grogginess, cognitive impairment, schizophrenia, spasticity associated with cerebral palsy, age-related memory decline, idiopathic somnolence, or jet-lag. Preferably, the combination method employs doses of modafinil in the range of about 20 to 300 mg per dose.

The compounds of the invention are used, alone or in combination with one or more other active ingredients, to formulate pharmaceutical compositions of the invention. A pharmaceutical composition of the invention comprises: (a) an effective amount of a compound of Formula (I), or a pharmaceutically acceptable salt, pharmaceutically acceptable prodrug, or pharmaceutically active metabolite thereof; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a compound of the invention and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the compounds of the invention may be prepared using suitable pharmaceutical excipients and compounding techniques now or later known or available to those skilled in the art. The compositions may be administered in the inventive methods by oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the invention can be provided in the form of tablets or capsules, or as a solution, emulsion, or suspension. To prepare the oral compositions, the compounds may be formulated to yield a dosage of, e.g., from about 0.01 to about 100 mg/kg daily, or from about 0.05 to about 35 mg/kg daily, or from about 0.1 to about 10 mg/kg daily.

Oral tablets may include a compound according to the invention mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, compounds of the invention may be mixed with a solid, semi-solid, or liquid diluent. Soft gelatin capsules may be prepared by mixing the compound of the invention with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compounds of this invention may also be administered by non-oral routes. For example, the compositions may be formulated for rectal administration as a suppository. For parenteral use, including intravenous, intramuscular, intraperitoneal, or subcutaneous routes, the compounds of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the invention may utilize a patch formulation to affect transdermal delivery.

Compounds of the invention may alternatively be administered in methods of this invention by inhalation, via the nasal or oral routes, e.g., in a spray formulation also containing a suitable carrier.

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Reactions may be performed between the melting point and the reflux temperature of the solvent, and preferably between 0° C. and the reflux temperature of the solvent. One skilled in the art will also recognize that it may be advantageous to perform the reactions shown in the Schemes in an order different from that depicted. Unless otherwise specified, the variables are as defined above in reference to Formula (I).

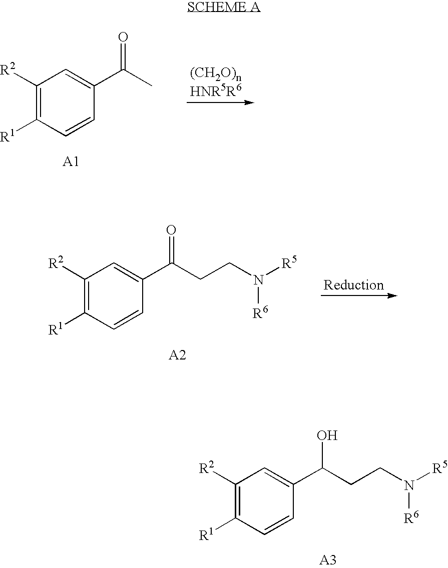

Referring to Scheme A, acetophenones A1 are condensed with paraformaldehyde (or an equivalent) in the presence of HNR⁵R⁶, a suitable acid catalyst such as HCl, in a solvent such as ethanol (EtOH), to form aminoketones A2. Aminoketones A2 are reduced to alcohols A3 using a hydride reagent such as NaBH₄ in a solvent such as EtOH.

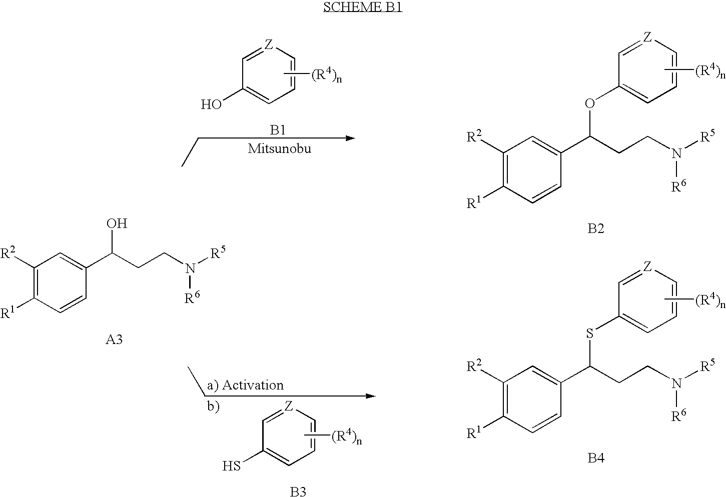

Referring to Scheme B1, conversion of alcohols A3 to ethers B2 is accomplished by a Mitsunobu reaction with aryl alcohols B1, in the presence of triphenylphosphine (optionally resin-bound) and a dialkyl-diazodicarboxylate reagent such as diethyl- or di-tert-butyl-azodicarboxylate, and in a solvent such as tetrahydrofuran (THF) or CH₂Cl₂. Thioethers B4 are prepared by activation activation of the secondary alcohol to a suitable leaving group, such as a mesylate, tosylate, or halide, followed in the same or a separate step by displacement with thiols B3. Alternatively, reaction of alcohols A3 with thiophenols B3 under Mitsunobu conditions provides thioethers B4 in one step.

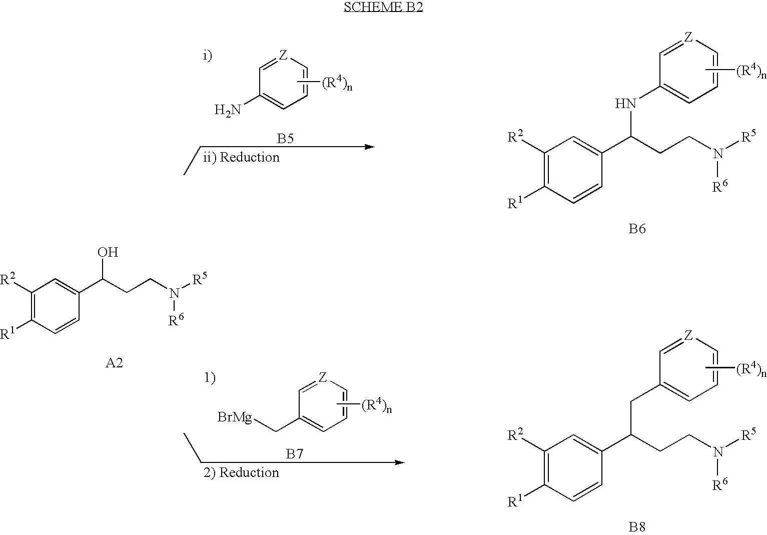

As depicted in Scheme B2, amines B6 are prepared by treating aminoketones A2 with amines B5 under reductive amination conditions known in the art. Preferred conditions include the use of a Lewis acid such as Ti(OiPr)₄, followed by the addition of a reducing agent such as NaBH₄, in a solvent such as methanol (MeOH) or EtOH. Carbon-linked analogs B8 are prepared by: (a) reaction of aminoketones A2 with an arylmethyl organometallic reagent, such as a Grignard reagent (B7), in a solvent such as THF or diethyl ether (Et₂O), at temperatures between about −78° C. and 0° C.; and either (b) treating the resulting tertiary alcohols (not shown) with H₂SO₄ and triethylsilane in a solvent such as trifluoroacetic acid (TFA). Where these procedures result in dehydration of the tertiary alcohol to an alkene (not shown), the alkene is reduced to the corresponding alkane B8 using standard method, such as hydrogenation.

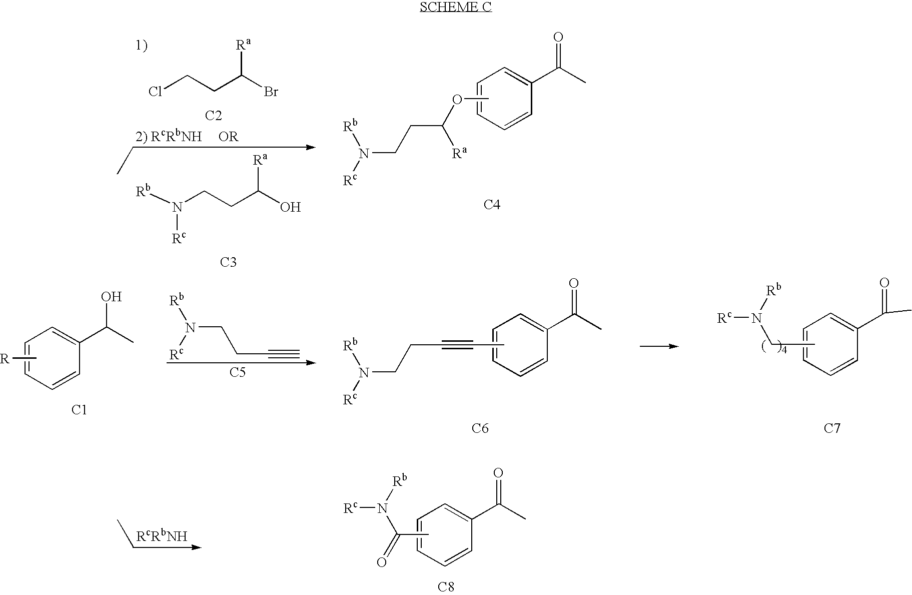

Groups “Q” in Formula (I) are installed as shown in Scheme C, starting from suitably substituted acetophenones C1. Where R is OH, acetophenones C1 are reacted with alkylating agents such as C2 in the presence of a suitable base such as K₂CO₃ or Na₂CO₃, in a solvent such as acetone, and at temperatures between about room temperature and the boiling point of the solvent. The resulting chloroethers (not shown) are converted into ethers C4 by reaction with amines HNR^{b}R^{c}, in the presence of a suitable base such as K₂CO₃ or Na₂CO₃, in a solvent such as n-butanol (n-BuOH), at temperatures between about room temperature and the boiling point of the solvent, and optionally with an activating agent such as NaI or KI. One skilled in the art will recognize this transformation may also be performed on a tosylate or mesylate (compounds C2 where Cl is replaced by methanesulfonyloxy or p-toluenesulfonyloxy), or by oxidation of a primary alcohol to the corresponding aldehyde followed by reductive amination with amines HNR^{b}R^{c}. Alternatively, ethers C4 are accessed by reaction of acetophenones C1 with pre-formed aminoalcohols C3 under Mitsunobu conditions such as those described for Scheme B1.

In another embodiment, where R is Br or I, acetophenones C1 are coupled with alkynes C5 in the presence of palladium catalyst such as PdCl₂(PPh₃)₂ or Pd(PPh₃)₄, a phosphine ligand such as PPh₃, optional additives such as copper(I) iodide and diethylamine, in a solvent such as N,N-dimethylformamide (DMF) or ethylene glycol dimethyl ether (DME), at temperatures between room temperature and the reflux temperature of the solvent, or using a high pressure reaction vessel or microwave reactor, to give alkynes C6. One skilled in the art will recognize that these palladium couplings may alternatively be performed using but-3-yn-1-ol, where the resulting hydroxyl group is subsequently converted into —NR^{b}R^{c} as described above. Alkynes C6 are reduced under hydrogenation conditions to give alkyl amines C7. Amides C8 are reduced to the corresponding benzyl amines C9 using a reducing agent such as LiAlH₄, preferably after the ketone group has been appropriately functionalized.

In another embodiment, where R is I, transition metal-catalyzed reaction of acetophenones C1 with amines HNR^{b}R^{c} and a CO equivalent, such as CO gas or Mo(CO)₆, in the presence of a suitable palladium (II) catalyst, such as Pd(OAc)₂, and optional additives such as 1,8-diazabicyclo[4.3.0]undec-7-ene (DBU) or (t-Bu)₃PHBF₄⁺, provides amides C8. Alternatively, where R is Br, halogen-metal exchange of acetophenones C1 by treatment with n-BuLi or t-BuLi, followed by quenching of the resulting anion with a CO₂ equivalent, provides the corresponding carboxylic acids (not shown). Amide coupling of such acids with amines HNR^{b}R^{c}, in the presence of coupling agents known to one skilled in the art, also provides amides C8. Where R is —CO₂H, some embodiments of acetophenones C1 are commercially available, and they are converted directly into amides C8 under peptide coupling conditions.

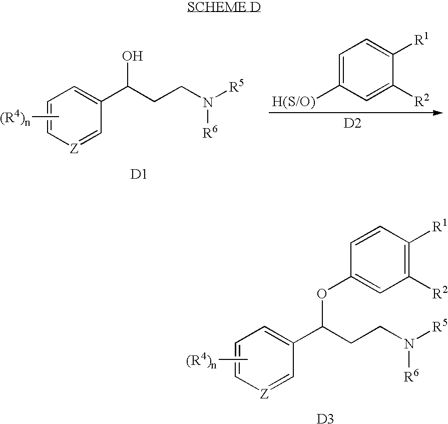

Referring to Scheme D, compounds D3 are prepared by Mitsunobu reaction of secondary alcohols D1 with phenols or thiophenols D2, using conditions such as those described in the preceding Schemes.

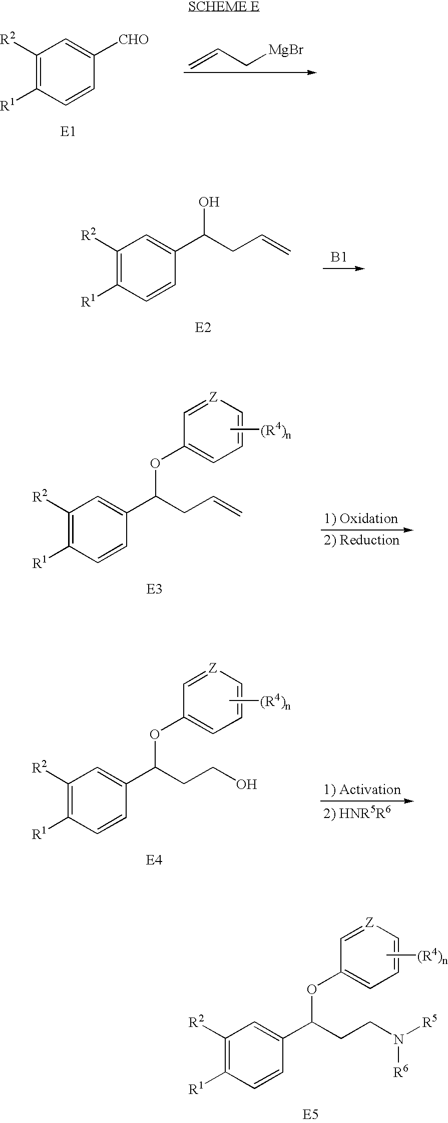

Referring to Scheme E, addition of an allyl organometallic reagent such as an allyl Grignard reagent to benzaldehydes E1 provides alcohols E2. Reactions are preferably performed in a solvent such as THF or Et₂O at temperatures between about −78° C. and 0° C. Oxidative cleavage of the double bond provides alcohols E4, and is accomplished by treatment with 03 followed by reduction of the resulting ozonide with, for example, NaBH₄ in MeOH. Activation of the alcohol and displacement with amines HNR⁵R⁶ as described in the preceding Schemes provides compounds E5.

Compounds of Formula (I) may be converted to their corresponding salts using methods known to those skilled in the art. For example, amines of Formula (I) may be treated with TFA, HCl, maleic acid, or citric acid in a solvent such as Et₂O, CH₂Cl₂, THF, or MeOH to provide the corresponding salt forms.

Compounds prepared according to the schemes described above may be obtained as single enantiomers, diastereomers, or regioisomers, by enantio-, diastero-, or regiospecific synthesis, or by resolution. Compounds prepared according to the schemes above may alternately be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation methods known to one skilled in the art, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using conventional methods such as chromatography or crystallization.

The following examples are provided to further illustrate the invention and various preferred embodiments.

### EXAMPLES

### Chemistry

In obtaining the characterization data described in the examples below, the following analytical and experimental protocols were followed as indicated.

Where solutions or mixtures were “concentrated”, they were typically concentrated under reduced pressure using a rotary evaporator. Where solutions were dried, they were generally dried over anhydrous Na₂SO₄. Reaction mixtures were magnetically stirred at room temperature (rt) unless otherwise specified.

Mass spectra were obtained on an Agilent series 1100 MSD using electrospray ionization (ESI) in positive mode unless otherwise indicated. Calcd. mass corresponds to the exact mass.

NMR spectra were obtained on either a Bruker model DPX400 (400 MHz), DPX500 (500 MHz), or DPX600 (600 MHz) spectrometer. The format of the ¹H NMR data below is: chemical shift in ppm down field of the tetramethylsilane reference (multiplicity, coupling constant J in Hz, integration).

Thin-layer chromatography was performed using Merck silica gel 60 F₂₅₄ 2.5 cm×7.5 cm 250 μm or 5.0 cm×10.0 cm 250 μm pre-coated silica gel plates. Preparative thin-layer chromatography was performed using EM Science silica gel 60 F₂₅₄ 20 cm×20 cm 0.5 mm pre-coated plates with a 20 cm×4 cm concentrating zone.

Unless otherwise specified, normal phase flash column chromatography (FCC) was typically performed with RediSep® silica gel columns using 2 M NH₃ in MeOH/CH₂Cl₂ as eluent.

Preparative Reverse Phase HPLC was performed under the following conditions: Instrument: Gilson®; Column: YMC-Pack ODS-A, 5 μm, 75×30 mm; flow rate: 25 mL/min; detection: λ=220 & 254 nm; gradient: 15% to 99% CH₃CN/H₂O (0.05% TFA) over 20 min.

### Example 1

### Dimethyl-[3-phenoxy-3-[4-(3-piperidin-1-yl-propoxy)-phenyl]-propyl]-amine maleic acid salt

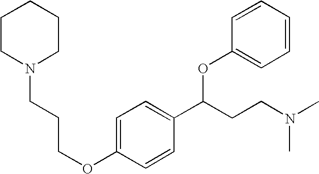

Step A: 1-[4-(3-Chloro-propoxy)-phenyl]-ethanone. A solution of 1-(4-hydroxy-phenyl)-ethanone (5.0 g, 37 mmol), K₂CO₃ (15.2 g, 110 mmol), and 1-bromo-3-chloro-propane (11.6 g, 73.4 mmol) in acetone (105 mL) was heated at reflux for 16 h. After cooling to room temperature (rt), a white solid was removed by filtration and the filtrate was concentrated. Purification of the residue by FCC (ethyl acetate (EtOAc)/hexanes) gave the desired product (7.6 g, 97%). MS (ESI): mass calcd. for C₁₁H₁₃Cl O₂, 212.06; m/z found, 213.1 [M+H]⁺. ¹H NMR (CDCl₃): 7.92 (d, J=8.8, 2H), 6.92 (d, J=8.8, 2H), 4.17 (t, J=5.9, 2H), 3.74 (t, J=7.8, 2H), 2.54 (s, 3H), 2.25 (quint, J=6.0, 2H).

Step B: 1-[4-(3-Piperidin-1-yl-propoxy)-phenyl]-ethanone. A mixture of 1-[4-(3-chloro-propoxy)-phenyl]-ethanone (7.37 g, 34.6 mmol), piperidine (4.43 g, 52.0 mmol), Na₂CO₃ (5.51 g, 52.0 mmol), and KI (287 mg, 1.73 mmol) in n-BuOH (42 mL) was heated at 105° C. for 36 h. After cooling to rt, the mixture was diluted with water and extracted with CH₂Cl₂. The combined organic layers were washed with satd. aq. NaCl, dried, and concentrated, and the residue was purified by FCC to give the desired product (9.07 g, 100%). MS (ESI): mass calcd. for C₁₆H₂₃NO₂, 261.17; m/z found, 262.2 [M+H]⁺. ¹H NMR (CDCl₃): 7.82 (d, J=7.2, 2H), 6.84 (d, J=7.4, 2H), 4.07-3.94 (m, 2H), 2.46 (s, 3H), 2.39 (t, J=6.4, 2H), 2.35-2.27 (m, 4H), 1.98-1.86 (m, 2H), 1.55-1.47 (m, 4H), 1.40-1.32 (m, 2H).

Step C: 3-Dimethylamino-1-[4-(3-piperidin-1-yl-propoxy)-phenyl]-propan-1-one. A mixture of 1-[4-(3-piperidin-1-yl-propoxy)-phenyl]-ethanone (3.0 g, 11 mmol), paraformaldehyde (452 mg, 14.9 mmol), HNMe₂.HCl (1.2 g, 15 mmol), conc. HCl (1.24 mL, 14.9 mmol) and EtOH (19 mL) was heated at reflux overnight.

Workup 1: After cooling to rt, the mixture was concentrated, diluted with satd. aq. NaHCO₃ and extracted with EtOAc. The combined organic layers were dried and concentrated. Purification by FCC gave the desired product (1.16 g, 32%). MS (ESI): mass calcd. for C₁₉H₃₀N₂O₂, 318.23; m/z found, 319.2 [M+H]⁺. ¹H NMR (CDCl₃): 7.93 (d, J=8.9, 2H), 6.93 (d, J=8.9, 2H), 4.08 (t, J=6.4, 2H), 3.11 (t, J=7.2, 2H), 2.75 (t, J=7.7, 2H), 2.48 (t, J=7.2, 2H), 2.45-2.35 (m, 4H), 2.29 (s, 6H), 2.03-1.95 (m, 2H), 1.62-1.56 (m, 4H), 1.48-1.40 (m, 2H).

Workup 2: Alternatively, after cooling to rt, the reaction mixture is diluted with acetone and filtered. Collection of the solid provides the desired product.

Step D: 3-Dimethylamino-1-[4-(3-piperidin-1-yl-propoxy)-phenyl]-propan-1-ol. To a solution of 3-dimethylamino-1-[4-(3-piperidin-1-yl-propoxy)phenyl]-propan-1-one (585 mg, 1.84 mmol) in EtOH (18.4 mL) was added NaBH₄ (139 mg, 3.68 mmol). After 18 h at rt, the mixture was diluted with water and extracted with Et₂O. The combined organic layers were dried and concentrated. Purification by FCC gave the desired product (434 mg, 73%). MS (ESI): mass calcd. for C₁₉H₃₂N₂O₂, 320.25; m/z found, 321.3 [M+H]⁺. ¹H NMR (CDCl₃): 7.19 (d, J=8.6, 2H), 6.78 (d, J=8.6, 2H), 4.77-4.73 (m, 1H), 3.90 (t, J=6.4, 2H), 2.57-2.49 (m, 1H), 2.42-2.37 (m, 7H), 2.19 (m, 6H), 1.92-1.84 (m, 2H), 1.74-1.67 (m, 2H), 1.55-1.47 (m, 4H), 1.40-1.31 (m, 2H).

Step E. A mixture of 3-dimethylamino-1-[4-(3-piperidin-1-yl-propoxy)phenyl]-propan-1-ol (380 mg, 1.19 mmol), phenol (167 mg, 1.78 mmol), resin bound PPh₃ (593 mg, 1.78 mmol), and di-tert-butyl azodicarboxylate (410 mg, 1.78 mmol) in CH₂Cl₂ (11.9 mL) was placed on a shaker apparatus under argon for 3 days. The resin was removed by filtration and the filtrate was concentrated. Purification of the residue by FCC gave the desired product (662 mg, 56%). MS (ESI): mass calcd. for C₂₅H₃₆N₂O₂, 396.28; m/z found, 397.2 [M+H]⁺. ¹H NMR (CDCl₃): 7.26 (d, J=8.6, 2H), 7.17 (t, J=7.8, 2H), 6.84 (d, J=8.7, 5H), 5.17-5.13 (m, 1H), 3.97 (t, J=6.4, 2H), 2.48-2.33 (m, 8H), 2.22 (s, 6H), 2.19-2.11 (m, 1H), 1.98-1.89 (m, 3H), 1.61-1.54 (m, 4H), 1.47-1.39 (m, 2H).

The compounds in Examples 2-19 were prepared according to the procedures described in Example 1. Where trifluoroacetatic acid salts were obtained, they were isolated by preparative reverse-phase HPLC.

### Example 2

### Dimethyl-[3-[4-(3-piperidin-1-yl-propoxy)-phenyl]-3-(4-trifluoromethyl-phenoxy)-propyl]-amine

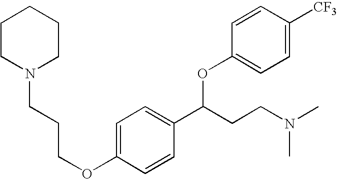

MS (ESI): mass calcd. for C₂₆H₃₅F₃N₂O₂, 464.27; m/z found, 465.2 [M+H]⁺. ¹H NMR (CDCl₃): 7.42 (d, J=8.6, 2H), 7.24 (d, J=8.7, 2H), 6.90 (d, J=8.6, 2H), 6.85 (d, J=8.7, 2H), 5.24-5.21 (m, 1H), 3.98 (t, J=6.4, 2H), 2.48-2.32 (m, 8H), 2.22 (s, 6H), 2.21-2.13 (m, 1H), 1.99-1.89 (m, 3H), 1.62-1.54 (m, 4H), 1.47-1.38 (m, 2H).

### Example 3

### Dimethyl-[3-phenoxy-3-[3-(3-piperidin-1-yl-propoxy)-phenyl]-propyl]-amine

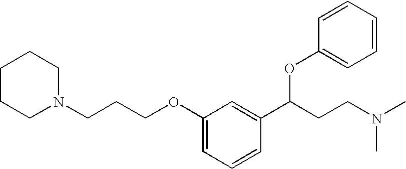

MS (ESI): mass calcd. for C₂₅H₃₆N₂O₂, 396.28; m/z found, 397.2 [M+H]⁺. ¹H NMR (CDCl₃): 7.23-7.13 (m, 3H), 6.95-6.90 (m, 2H), 6.89-6.82 (m, 3H), 6.80-6.74 (m, 1H), 5.17-5.13 (m, 1H), 3.96 (td, J=2.3, 6.3, 2H), 2.48-2.33 (m, 8H), 2.22 (s, 6H), 2.19-2.09 (m, 1H), 1.98-1.89 (m, 3H), 1.62-1.54 (m, 4H), 1.47-1.39 (m, 2H). The free base was converted to the maleic acid salt for biological testing.

### Example 4

### Dimethyl-{3-(4-methylsulfanyl-phenoxy)-3-[4-(3-piperidin-1-yl-propoxy)-phenyl]-propyl}-amine trifluoroacetic acid salt

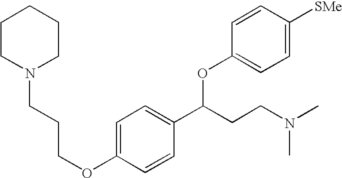

MS (ESI): mass calcd. for C₂₆H₃₈N₂O₂S, 442.27; m/z found, 443.3 [M+H]⁺. ¹H NMR (acetone-d₆): 7.37 (d, J=8.7, 2H), 7.16 (d, J=8.9, 2H), 6.92 (d, J=8.7, 2H), 6.89 (d, J=8.8, 2H), 5.45-5.41 (m, 1H), 4.10 (t, J=6.0, 2H), 3.67-3.58 (m, 2H), 3.44-3.26 (m, 4H), 2.99-2.90 (m, 2H), 2.92 (s, 6H), 2.51-2.42 (m, 1H), 2.39 (s, 3H), 2.36-2.26 (m, 3H), 2.00-1.76 (m, 5H), 1.57-1.44 (m, 1H).

### Example 5

### 4-{3-Dimethylamino-1-[4-(3-piperidin-1-yl-propoxy)-phenyl]-propoxy}-benzonitrile trifluoroacetic acid salt

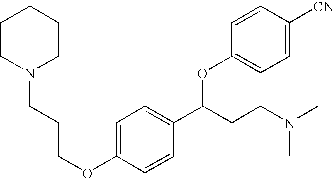

MS (ESI): mass calcd. for C₂₆H₃₅N₃O₂, 421.27; m/z found, 422.2 [M+H]⁺. ¹H NMR (MeOD): 7.56 (d, J=9.0, 2H), 7.37 (d, J=8.7, 2H), 7.03 (d, J=8.9, 2H), 6.96 (d, J=8.7, 2H), 5.50-5.46 (m, 1H), 4.10 (t, J=5.7, 2H), 3.60 (d, J=12.2, 2H), 3.44-3.36 (m, 1H), 3.34-3.27 (m, 3H), 3.00-2.95 (m, 2H), 2.93 (s, 6H), 2.50-2.41 (m, 1H), 2.34-2.19 (m, 3H), 2.01-1.94 (m, 2H), 1.90-1.73 (m, 3H), 1.59-1.48 (m, 1H).

### Example 6

### Dimethyl-{3-[4-(3-piperidin-1-yl-propoxy)-phenyl]-3-p-tolyloxy-propyl}-amine trifluoroacetic acid salt

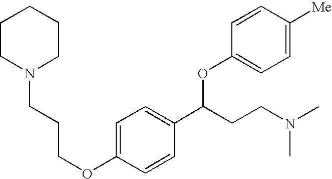

MS (ESI): mass calcd. for C₂₆H₃₈N₂O₂, 410.29; m/z found, 411.2 [M+H]⁺. ¹H NMR (acetone-d₆): 7.36 (d, J=8.7, 2H), 6.99 (d, J=8.3, 2H), 6.91 (d, J=8.7, 2H), 6.79 (d, J=8.6, 2H), 5.41-5.36 (m, 1H), 4.09 (t, J=6.1, 2H), 3.66-3.57 (m, 2H), 3.41-3.24 (m, 4H), 2.98-2.90 (m, 2H), 2.91 (s, 6H), 2.49-2.40 (m, 1H), 2.35-2.25 (m, 3H), 2.19 (s, 3H), 1.99-1.77 (m, 5H), 1.56-1.45 (m, 1H).

### Example 7

### {3-(4-Methoxy-phenoxy)-3-[4-(3-piperidin-1-yl-propoxy)-phenyl]-propyl}-dimethyl-amine trifluoroacetic acid salt

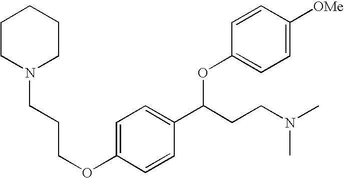

MS (ESI): mass calcd. for C₂₆H₃₈N₂O₃, 426.29; m/z found, 427.3 [M+H]⁺. ¹H NMR (acetone-d₆): 7.35 (d, J=8.6, 2H), 6.91 (d, J=8.6, 2H), 6.84 (d, J=9.1, 2H), 6.75 (d, J=9.0, 2H), 5.35-5.29 (m, 1H), 4.11 (t, J=5.9, 2H), 3.70-3.62 (m, 5H), 3.48-3.29 (m, 4H), 3.04-2.93 (m, 8H), 2.50-2.41 (m, 1H), 2.35-2.26 (m, 3H), 1.95-1.86 (m, 4H), 1.84-1.77 (m, 1H), 1.58-1.46 (m, 1H).

### Example 8

### {3-(4-Chloro-phenoxy)-3-[4-(3-piperidin-1-yl-propoxy)-phenyl]-propyl}-dimethyl-amine trifluoroacetic acid salt

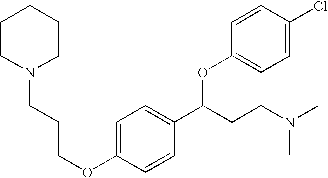

MS (ESI): mass calcd. for C₂₅H₃₅ClN₂O₂, 430.24; m/z found, 431.2 [M+H]⁺. ¹H NMR (MeOD): 7.35 (d, J=8.7, 2H), 7.16 (d, J=9.0, 2H), 6.95 (d, J=8.7, 2H), 6.86 (d, J=9.0, 2H), 5.35-5.31 (m, 1H), 4.10 (d, J=5.6, 2H), 3.59 (d, J=12.1, 2H), 3.50-3.30 (m, 4H), 3.00-2.91 (m, 8H), 2.45-2.36 (m, 1H), 2.29-2.19 (m, 3H), 1.97 (d, J=14.4, 2H), 1.90-1.73 (m, 3H), 1.59-1.48 (m, 1H).

### Example 9

### {3-(3,4-Dichloro-phenoxy)-3-[4-(3-piperidin-1-yl-propoxy)-phenyl]-propyl}-dimethyl-amine

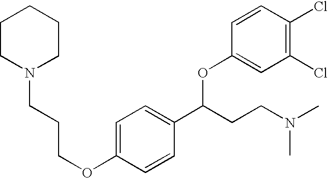

MS (ESI): mass calcd. for C₂₅H₃₄Cl₂N₂O₂, 464.20; m/z found, 465.2 [M+H]⁺. ¹H NMR (acetone-d₆): 7.41-7.35 (m, 3H), 7.13 (d, J=2.8, 1H), 6.96-6.91 (m, 3H), 5.55-5.51 (m, 1H), 4.11 (t, J=6.0, 2H), 3.65-3.60 (m, 2H), 3.43-3.26 (m, 4H), 2.99-2.91 (m, 8H), 2.55-2.45 (m, 1H), 2.40-2.26 (m, 3H), 2.00-1.77 (m, 5H), 1.57-1.46 (m, 1H). The free base was converted to the maleic acid salt for biological testing.

### Example 10

### {3-(4-Fluoro-phenoxy)-3-[4-(3-piperidin-1-yl-propoxy)-phenyl]-propyl}-dimethyl-amine trifluoroacetic acid salt

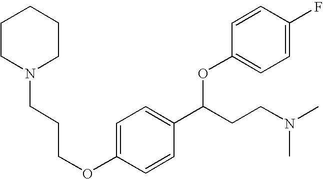

MS (ESI): mass calcd. for C₂₅H₃₅FN₂O₂, 414.27; m/z found, 415.2 [M+H]⁺. ¹H NMR (acetone-d₆): 7.34 (d, J=8.7, 2H), 6.96-6.88 (m, 6H), 5.39-5.35 (m, 1H), 4.08 (t, J=6.1, 2H), 3.41-2.90 (m, 8H), 2.82 (s, 6H), 2.46-2.37 (m, 1H), 2.31-2.21 (m, 3H), 1.89-1.83 (m, 4H), 1.75-1.55 (m, 2H).

### Example 11

### {3-(4-Bromo-phenoxy)-3-[4-(3-piperidin-1-yl-propoxy)-phenyl]-propyl}-dimethyl-amine trifluoroacetic acid salt

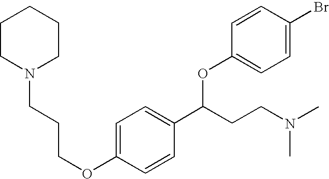

MS (ESI): mass calcd. for C₂₅H₃₅BrN₂O₂, 474.19; m/z found, 475.3, 477.3 [M+H]⁺. ¹H NMR (MeOD): 7.32 (d, J=8.6, 2H), 7.27 (d, J=8.9, 2H), 6.92 (d, J=8.6, 2H), 6.79 (d, J=8.9, 2H), 5.36-5.28 (m, 1H), 4.07 (t, J=5.6, 2H), 3.63-3.54 (m, 2H), 3.42-3.24 (m, 4H), 3.01-2.88 (m, 8H), 2.44-2.33 (m, 1H), 2.30-2.16 (m, 3H), 2.01-1.90 (m, 2H), 1.90-1.70 (m, 3H), 1.58-1.46 (m, 1H).

### Example 12

### Dimethyl-{3-(4-nitro-phenoxy)-3-[4-(3-piperidin-1-yl-propoxy)phenyl]-propyl}-amine trifluoroacetic acid salt

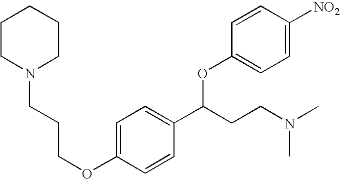

MS (ESI): mass calcd. for C₂₅H₃₅N₃O₄, 441.26; m/z found, 442.4 [M+H]⁺. ¹H NMR (MeOD): 8.12 (d, J=9.2, 2H), 7.33 (d, J=8.6, 2H), 6.94 (d, J=8.7, 2H), 6.89 (d, J=9.2, 2H), 4.78-4.74 (m, 1H), 4.09 (t, J=5.7, 2H), 3.62-3.57 (m, 2H), 3.32-3.28 (m, 2H), 3.22-3.16 (m, 1H), 2.99-2.93 (m, 2H), 2.93-2.91 (m, 1H), 2.90 (s, 6H), 2.25-2.18 (m, 2H), 2.12-2.06 (m, 1H), 2.06-2.00 (m, 1H), 2.00-1.93 (m, 2H), 1.88-1.82 (m, 1H), 1.81-1.71 (m, 2H), 1.58-1.48 (m, 1H).

### Example 13

### {3-(3-Methoxy-phenoxy)-3-[4-(3-piperidin-1-yl-propoxy)-phenyl]-propyl}-dimethyl-amine trifluoroacetic acid salt

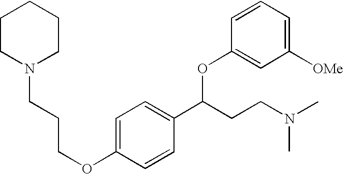

MS (ESI): mass calcd. for C₂₆H₃₈N₂O₃, 426.29; m/z found, 427.3 [M+H]⁺. ¹H NMR (MeOD): 7.24 (d, J=8.7, 2H), 6.95 (t, J=8.3, 1H), 6.83 (d, J=8.7, 2H), 6.36-6.31 (m, 3H), 5.23-5.19 (m, 1H), 3.98 (t, J=5.7, 2H), 3.58 (s, 3H), 3.50-3.44 (m, 2H), 3.31-3.24 (m, 1H), 3.20-3.14 (m, 3H), 2.87-2.82 (m, 2H), 2.80 (s, 6H), 2.30-2.23 (m, 1H), 2.16-2.06 (m, 3H), 1.88-1.81 (m, 2H), 1.77-1.61 (m, 3H), 1.47-1.36 (m, 1H).

### Example 14

### {3-(3-Chloro-phenoxy)-3-[4-(3-piperidin-1-yl-propoxy)-phenyl]-propyl}-dimethyl-amine trifluoroacetic acid salt

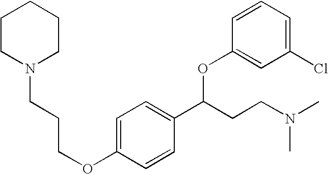

MS (ESI): mass calcd. for C₂₅H₃₅ClN₂O₂, 430.24; m/z found, 431.2 [M+H]⁺. ¹H NMR (CDCl₃): 12.61 (bs, 1H), 11.63 (bs, 1H), 7.21 (d, J=8.7, 2H), 7.10 (t, J=8.2, 1H), 6.89-6.85 (m, 1H), 6.84-6.79 (m, 3H), 6.69-6.65 (m, 1H), 5.25-5.19 (m, 1H), 4.04 (t, J=5.5, 2H), 3.67 (d, J=11.8, 2H), 3.25-3.13 (m, 4H), 2.83 (d, J=10.5, 6H), 2.71-2.60 (m, 2H), 2.41-2.20 (m, 4H), 2.05-1.85 (m, 5H), 1.48-1.35 (m, 1H).

### Example 15

### {3-(3-Bromo-phenoxy)-3-[4-(3-piperidin-1-yl-propoxy)-phenyl]-propyl}-dimethyl-amine

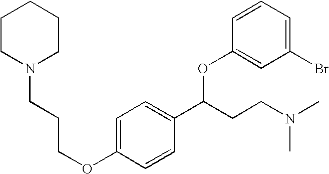

MS (ESI): mass calcd. for C₂₅H₃₅BrN₂O₂, 474.19; m/z found, 475.4, 477.4 [M+H]⁺. ¹H NMR (CDCl₃): 7.23 (d, J=8.7, 2H), 7.05-6.95 (m, 3H), 6.85 (d, J=8.7, 2H), 6.77-6.72 (m, 1H), 5.16-5.11 (m, 1H), 3.98 (t, J=6.4, 2H), 2.48-2.34 (m, 8H), 2.22 (s, 6H), 2.19-2.04 (m, 1H), 1.94-1.85 (m, 3H), 1.60 (quint, J=5.8, 4H), 1.47-1.39 (m, 2H).

### Example 16

### {3-(2,4-Dichloro-phenoxy)-3-[4-(3-piperidin-1-yl-propoxy)-phenyl]-propyl}-dimethyl-amine

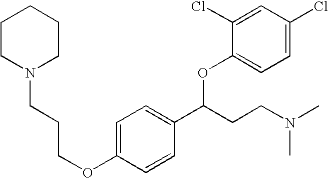

MS (ESI): mass calcd. for C₂₅H₃₄Cl₂N₂O₂, 464.20; m/z found, 465.3 [M+H]⁺. ¹H NMR (MeOD): 7.35 (d, J=2.6, 1H), 7.27 (d, J=8.7, 2H), 7.04 (dd, J=2.6, 8.9, 1H), 6.88 (d, J=8.7, 2H), 6.83 (d, J=8.9, 1H), 5.31-5.28 (m, 1H), 3.98 (t, J=6.1, 2H), 2.58-2.43 (m, 8H), 2.24 (s, 6H), 2.23-2.17 (m, 1H), 2.05-1.93 (m, 3H), 1.61 (quint, J=5.6, 4H), 1.50-1.44 (m, 2H).

### Example 17

### {3-(2-Chloro-phenoxy)-3-[4-(3-piperidin-1-yl-propoxy)-phenyl]-propyl}-dimethyl-amine trifluoroacetic acid salt

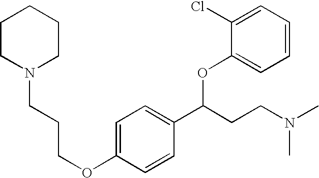

MS (ESI): mass calcd. for C₂₅H₃₅ClN₂O₂, 430.24; m/z found, 431.4 [M+H]⁺. ¹H NMR (MeOD): 7.33 (d, J=8.6, 2H), 7.27 (dd, J=1.6, 8.0, 1H), 7.12-7.08 (m, 1H), 6.94 (d, J=8.7, 2H), 6.89 (dd, J=1.4, 8.1, 1H), 6.80 (td, J=1.5, 7.9, 1H), 4.78-4.74 (m, 1H), 4.09 (t, J=5.7, 2H), 3.59 (d, J=11.9, 2H), 3.21-3.15 (m, 1H), 2.99-2.92 (m, 2H), 2.90 (s, 6H), 2.86-2.80 (m, 1H), 2.26-2.18 (m, 3H), 2.12-2.06 (m, 1H), 2.05-2.00 (m, 1H), 2.00-1.93 (m, 3H), 1.88-1.82 (m, 1H), 1.81-1.72 (m, 2H), 1.57-1.48 (m, 1H).

### Example 18

### Dimethyl-[3-[4-(3-piperidin-1-yl-propoxy)-phenyl]-3-(pyridin-3-yloxy)-propyl]-amine

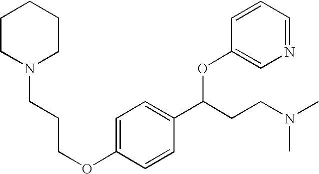

MS (ESI): mass calcd. for C₂₄H₃₅N₃O₂, 397.27; m/z found, 431.4 [M+H]⁺. ¹H NMR (CDCl₃): 8.30-8.23 (m, 1H), 8.14-8.06 (m, 1H), 7.28-7.22 (m, 2H), 7.11-7.05 (m, 2H), 6.87-6.82 (m, 2H), 5.18 (dd, J=7.8, 5.4, 1H), 3.96 (t, J=6.4, 6.4, 2H), 2.48-2.44 (m, 2H), 2.43-2.35 (m, 6H), 2.23 (s, 6H), 2.22-2.14 (m, 1H), 2.00-1.90 (m, 3H), 1.62-1.55 (m, 4H), 1.48-1.39 (m, 2H).

### Example 19

### {3-(1H-Indol-5-yloxy)-3-[4-(3-piperidin-1-yl-propoxy)-phenyl]-propyl}-dimethyl-amine trifluoroacetic acid salt

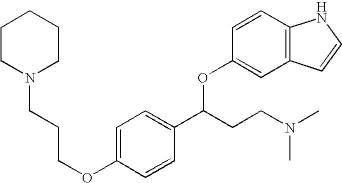

MS (ESI): mass calcd. for C₂₇H₃₇N₃O₂, 435.29; m/z found, 431.4 [M+H]⁺.

### Example 20

### Dimethyl-[3-[4-(4-piperidin-1-yl-but-1-ynyl)-phenyl]-3-(4-trifluoromethyl-phenoxy)-propyl]-amine

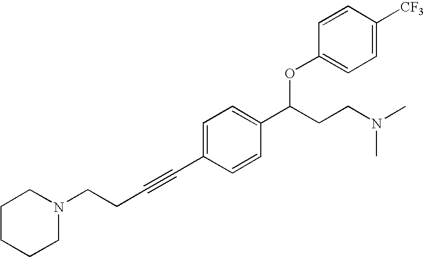

Step A: 1-(4-Bromo-phenyl)-3-dimethylamino-propan-1-one. The title compound was prepared according to Example 1, Step C, using workup 2. MS (ESI): mass calcd. for C₁₁H₁₄BrNO, 255.03; m/z found, 256.0, 258.0 [M+H]⁺. ¹H NMR (DMSO): 7.95 (d, J=8.5, 2H), 7.79 (d, J=8.5, 2H), 3.65 (t, J=7.2, 2H), 3.39 (t, J=7.5, 2H), 2.79 (s, 6H).

Step B: 1-(4-Bromo-phenyl)-3-dimethylamino-propan-1-ol. The title compound was prepared according to Example 1, Step D. MS (ESI): mass calcd. for C₁₁H₁₆BrNO, 257.04; m/z found, 258.0, 260.0 [M+H]⁺. ¹H NMR (CDCl₃): 7.46 (d, J=8.4, 2H), 7.26 (d, J=8.4, 2H), 4.91-4.87 (m, 1H), 2.70-2.62 (m, 1H), 2.49-2.43 (m, 1H), 2.29 (s, 6H), 1.85-1.69 (m, 2H).

Step C: [3-(4-Bromo-phenyl)-3-phenoxy-propyl]-dimethyl-amine. The title compound was prepared according to Example 1, Step E. MS (ESI): mass calcd. for C₁₇H₂₀BrNO, 333.07; m/z found, 334.0, 336.0 [M+H]⁺. ¹H NMR (CDCl₃): 7.44 (d, J=8.3, 2H), 7.24 (d, J=8.2, 2H), 7.18 (t, J=7.9, 2H), 6.87 (t, J=7.3, 1H), 6.82 (d, J=7.9, 2H), 5.22-5.16 (m, 1H), 2.43-2.34 (m, 2H), 2.22 (s, 6H), 2.20-2.09 (m, 1H), 1.95-1.85 (m, 1H).

Step D. To a sealed tube were added [3-(4-bromo-phenyl)-3-phenoxy-propyl]-dimethyl-amine (162 mg, 0.484 mmol), 1-but-3-ynyl-piperidine (99.8 mg, 0.727 mmol), Pd(PPh₃)₂Cl₂ (18.7 mg, 0.0266 mmol), CuI (5.1 mg, 0.027 mmol), HNEt₂ (3.23 mL, 32.2 mmol) and DMF (0.97 mL). The reaction mixture was heated in a microwave apparatus at 120° C. for 25 min. After cooling to rt, the mixture was filtered though diatomaceous earth, rinsing with Et₂O. The filtrate was concentrated and the residue purified by FCC to give the desired product (117 mg, 39%). MS (ESI): mass calcd. for C₂₇H₃₃F₃N₂O, 458.25; m/z found, 459.2 [M+H]⁺. ¹H NMR (CDCl₃): 7.42 (d, J=8.8, 2H), 7.36 (d, J=8.2, 2H), 7.26 (d, J=7.9, 2H), 6.88 (d, J=8.7, 2H), 5.28-5.23 (m, 1H), 2.67-2.54 (m, 4H), 2.50-2.30 (m, 5H), 2.22 (s, 6H), 2.20-2.11 (m, 2H), 1.98-1.88 (m, 1H), 1.60 (quint, J=5.7, 4H), 1.48-1.40 (m, 2H).

The compounds in Examples 21-31 were prepared as described in Example 20.

### Example 21

### Dimethyl-{3-phenoxy-3-[4-(4-piperidin-1-yl-but-1-ynyl)-phenyl]-propyl}-amine

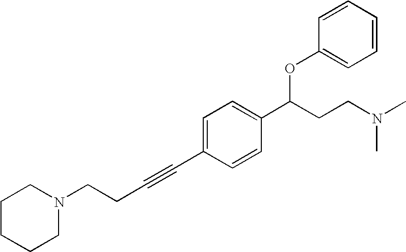

MS (ESI): mass calcd. for C₂₆H₃₄N₂O, 390.27; m/z found, 391.3 [M+H]⁺. ¹H NMR (CDCl₃): 7.34 (d, J=8.2, 2H), 7.27 (d, J=7.9, 2H), 7.19-7.13 (m, 2H), 6.88-6.79 (m, 3H), 5.21-5.16 (m, 1H), 2.67-2.55 (m, 4H), 2.48-2.36 (m, 5H), 2.21 (s, 6H), 2.20-2.08 (m, 2H), 1.96-1.87 (m, 1H), 1.60 (quint, J=5.7, 4H), 1.47-1.40 (m, 2H). The free base was converted to the maleic acid salt for biological testing.

### Example 22

### (3-{4-[4-(4-Isopropyl-piperazin-1-yl)-but-1-ynyl]-phenyl}-3-phenoxy-propyl)-dimethyl-amine

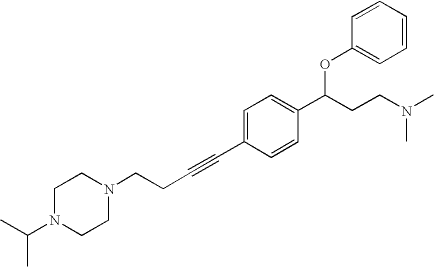

MS (ESI): mass calcd. for C₂₈H₃₉N₃O, 433.31; m/z found, 434.3 [M+H]⁺. ¹H NMR (CDCl₃): 7.34 (d, J=8.2, 2H), 7.29-7.25 (m, 2H), 7.18 (dd, J=8.4, 7.5, 2H), 6.89 (t, J=7.4, 1H), 6.79 (d, J=8.0, 2H), 5.24 (dd, J=8.2, 4.2, 1H), 3.37-3.24 (m, 1H), 3.19-2.77 (m, 10H), 2.72 (t, J=7.1, 2H), 2.63 (s, 6H), 2.58 (t, J=7.1, 2H), 2.34-2.14 (m, 2H), 1.28 (d, J=6.6, 6H).

### Example 23

### Dimethyl-{3-[4-(4-morpholin-4-yl-but-1-ynyl)-phenyl]-3-phenoxy-propyl}-amine

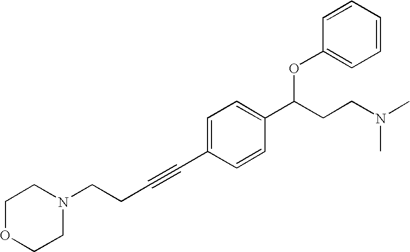

MS (ESI): mass calcd. for C₂₅H₃₂N₂O₂, 392.25; m/z found, 393.2 [M+H]⁺. ¹H NMR (CDCl₃): 7.35 (d, J=8.2, 2H), 7.29-7.25 (m, 2H), 7.21-7.15 (m, 2H), 6.92-6.86 (m, 1H), 6.79 (d, J=7.9, 2H), 5.27-5.22 (m, 1H), 3.76-3.71 (m, 4H), 3.06-2.89 (m, 2H), 2.69-2.56 (m, 10H), 2.56-2.50 (m, 4H), 2.34-2.15 (m, 2H).

### Example 24

### Dimethyl-{3-phenoxy-3-[4-(4-thiomorpholin-4-yl-but-1-ynyl)phenyl]-propyl}-amine

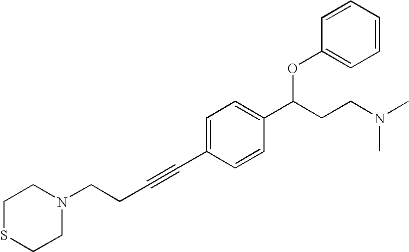

MS (ESI): mass calcd. for C₂₅H₃₂N₂OS, 408.22; m/z found, 409.2 [M+H]⁺. ¹H NMR (CDCl₃): 7.35 (d, J=8.2, 2H), 7.26 (t, J=4.1, 1H), 7.17 (dd, J=8.6, 7.5, 2H), 6.89 (t, J=7.4, 1H), 6.78 (d, J=7.9, 2H), 5.24 (dd, J=8.2, 4.3, 1H), 3.12-2.92 (m, 2H), 2.85-2.77 (m, 4H), 2.75-2.60 (m, 13H), 2.56 (t, J=7.4, 2H), 2.36-2.17 (m, 2H).

### Example 25

### {3-(4-Chloro-phenoxy)-3-[4-(4-piperidin-1-yl-but-1-ynyl)-phenyl]-propyl}-dimethyl-amine

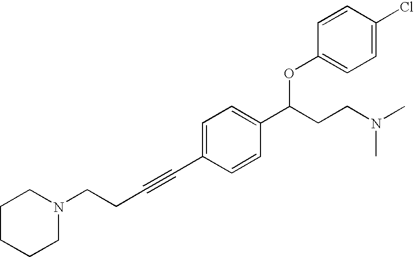

MS (ESI): mass calcd. for C₂₆H₃₃ClN₂O, 424.23; m/z found, 425.2 [M+H]⁺. ¹H NMR (CDCl₃): 7.34 (d, J=8.3, 2H), 7.24 (d, J=8.2, 2H), 7.14-7.09 (m, 2H), 6.75-6.68 (m, 2H), 5.20 (dd, J=8.2, 4.4, 1H), 3.02-2.71 (m, 10H), 2.53 (s, 6H), 2.29-2.06 (m, 2H), 1.83-1.74 (m, 4H), 1.61-1.49 (m, 2H).

### Example 26

### {3-(4-Methoxy-phenoxy)-3-[4-(4-thiomorpholin-4-yl-but-1-ynyl)phenyl]-propyl}-dimethyl-amine

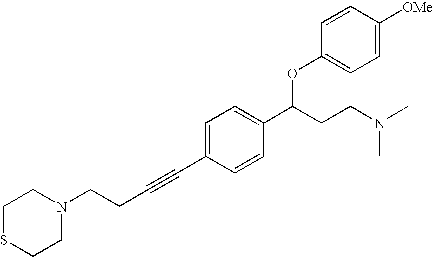

MS (ESI): mass calcd. for C₂₆H₃₄N₂O₂S, 438.23; m/z found, 439.2 [M+H]⁺. ¹H NMR (CDCl₃): 7.35 (d, J=8.3, 2H), 7.24 (d, J=8.3, 2H), 6.74-6.69 (m, 4H), 5.18-5.13 (m, 1H), 3.71 (s, 3H), 3.29-3.08 (m, 2H), 2.92-2.68 (m, 16H), 2.63-2.56 (m, 2H), 2.36-2.24 (m, 2H).

### Example 27

### Dimethyl-[3-[4-(4-morpholin-4-yl-but-1-ynyl)-phenyl]-3-(4-trifluoromethyl-phenoxy)-propyl]-amine

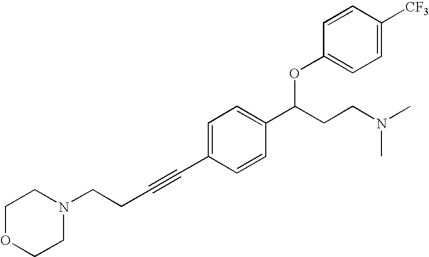

MS (ESI): mass calcd. for C₂₆H₃₁F₃N₂O₂, 460.23; m/z found, 461.2 [M+H]⁺. ¹H NMR (CDCl₃): 7.42 (d, J=8.8, 2H), 7.36 (d, J=8.2, 2H), 7.25 (d, J=7.8, 2H), 6.86 (d, J=8.7, 2H), 5.29 (dd, J=8.2, 4.60, 1H), 3.74-3.70 (m, 4H), 2.79-2.55 (m, 6H), 2.54-2.50 (m, 4H), 2.45 (s, 6H), 2.30-2.18 (m, 1H), 2.15-2.02 (m, 1H).

### Example 28

### Dimethyl-[3-[4-(4-thiomorpholin-4-yl-but-1-ynyl)-phenyl]-3-(4-trifluoromethyl-phenoxy)-propyl]-amine

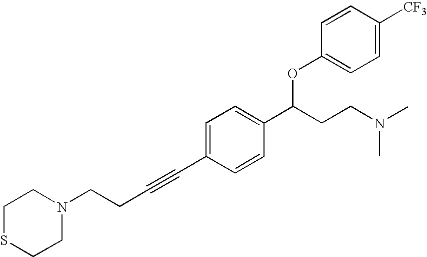

MS (ESI): mass calcd. for C₂₆H₃₁F₃N₂OS, 476.21; m/z found, 477.2 [M+H]⁺. ¹H NMR (CDCl₃): 7.42 (d, J=8.6, 2H), 7.35 (d, J=8.3, 2H), 7.25 (d, J=7.9, 2H), 6.86 (d, J=8.6, 2H), 5.29 (dd, J=8.2, 4.6, 1H), 2.82-2.77 (m, 4H), 2.76-2.60 (m, 8H), 2.56 (t, J=7.4, 2H), 2.44 (s, 6H), 2.29-2.17 (m, 1H), 2.13-2.01 (m, 1H).

### Example 29

### [3-{4-[4-(4-Isopropyl-piperazin-1-yl)-but-1-ynyl]-phenyl}-3-(4-trifluoromethyl-phenoxy)-propyl]-dimethyl-amine

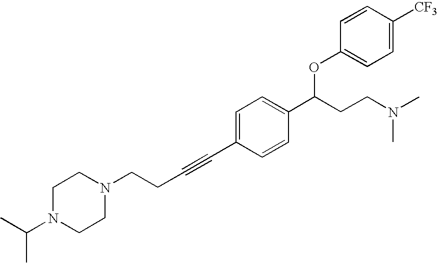

MS (ESI): mass calcd. for C₂₉H₃₈F₃N₃OS, 501.30; m/z found, 502.3 [M+H]⁺. ¹H NMR (CDCl₃): 7.43 (d, J=8.6, 2H), 7.35 (d, J=8.3, 2H), 7.27-7.23 (m, 1H), 6.87 (d, J=8.5, 2H), 5.30-5.27 (m, 1H), 3.28-3.13 (m, 1H), 3.08-2.62 (m, 12H), 2.61-2.56 (m, 2H), 2.50-2.41 (m, 6H), 2.30-2.19 (m, 1H), 2.15-2.04 (m, 1H), 1.28-1.22 (m, 7H).

### Example 30

### {3-(3,4-Dichloro-phenoxy)-3-[4-(4-piperidin-1-yl-but-1-ynyl)phenyl]-propyl}-dimethyl-amine trifluoroacetic acid salt

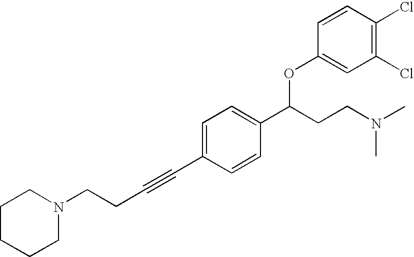

MS (ESI): mass calcd. for C₂₆H₃₂Cl₂N₂O, 458.19; m/z found, 459.4 [M+H]⁺.

### Example 31

### Dimethyl-{3-phenoxy-3-[3-(4-piperidin-1-yl-but-1-ynyl)-phenyl]-propyl}-amine trifluoroacetic acid salt

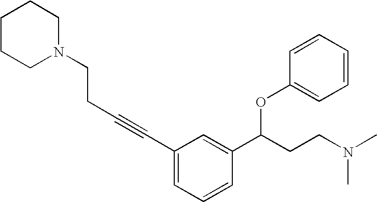

MS (ESI): mass calcd. for C₂₆H₃₄N₂O, 390.27; m/z found, 391.2 [M+H]⁺. ¹H NMR (MeOD): 7.36 (s, 1H), 7.33-7.31 (m, 1H), 7.27-7.24 (m, 2H), 7.11-7.07 (m, 2H), 6.80-6.74 (m, 3H), 5.29-5.26 (m, 1H), 3.52-3.49 (m, 2H), 3.27-3.24 (m, 2H), 3.22-3.20 (m, 2H), 2.93-2.88 (m, 2H), 2.86-2.82 (m, 8H), 2.30-2.22 (m, 1H), 2.20-2.13 (m, 1H), 1.87-1.84 (m, 2H), 12.75-1.63 (m, 3H), 1.47-1.37 (m, 1H).

### Example 32

### Dimethyl-{3-phenoxy-3-[4-(4-piperidin-1-yl-butyl)-phenyl]-propyl}-amine

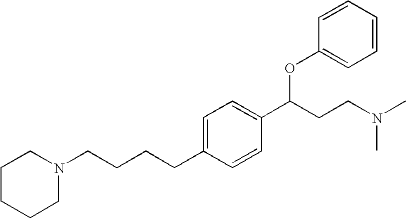

To a degassed solution of dimethyl-{3-phenoxy-3-[4-(4-piperidin-1-yl-but-1-ynyl)-phenyl]-propyl}-amine (64.1 mg, 0.164 mmol) in MeOH (1 mL) and EtOH (1 mL) was added 5% Pd on BaSO₄ (22 mg). A H₂ balloon was fitted to the flask and the reaction was stirred overnight. The reaction mixture was filtered through diatomaceous earth, the filtrate was concentrated, and the residue purified by FCC to give the desired product (51.6 mg, 80%). MS (ESI): mass calcd. for C₂₆H₃₈N₂O, 394.30; m/z found, 395.6 [M+H]⁺. ¹H NMR (CDCl₃): 7.32-7.28 (m, 2H), 7.24-7.14 (m, 4H), 6.92-6.86 (m, 3H), 5.24-5.20 (m, 1H), 2.64 (t, J=7.0, 2H), 2.48-2.32 (m, 8H), 2.27 (s, 6H), 2.24-2.15 (m, 1H), 2.03-1.93 (m, 1H), 1.68-1.52 (m, 8H), 1.51-1.42 (m, 2H).

The compounds in Examples 33-35 were prepared as in Example 32.

### Example 33

### Dimethyl-[3-[4-(4-piperidin-1-yl-butyl)-phenyl]-3-(4-trifluoromethylphenoxy)-propyl]-amine

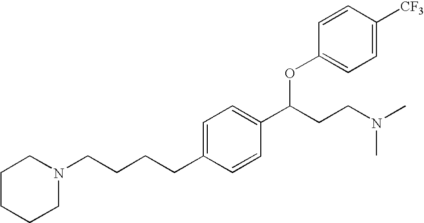

MS (ESI): mass calcd. for C₂₇H₃₇F₃N₂O, 462.29; m/z found, 463.5 [M+H]⁺. ¹H NMR (CDCl₃): 7.41 (d, J=8.5, 2H), 7.23 (d, J=8.1, 2H), 7.13 (d, J=8.1, 2H), 6.90 (d, J=8.6, 2H), 5.23 (dd, J=8.1, 5.0, 1H), 2.59 (t, J=7.4, 2H), 2.43-2.25 (m, 8H), 2.22 (s, 6H), 2.20-2.11 (m, 1H), 1.98-1.90 (m, 1H), 1.63-1.47 (m, 8H), 1.46-1.37 (m, 2H). The free base was converted to the maleic acid salt for biological testing.

### Example 34

### [3-[4-[4-(4-Isopropyl-piperazin-1-yl)-butyl]-phenyl]-3-(4-trifluoromethyl-phenoxy)-propyl]-dimethyl-amine trifluoroacetic acid salt

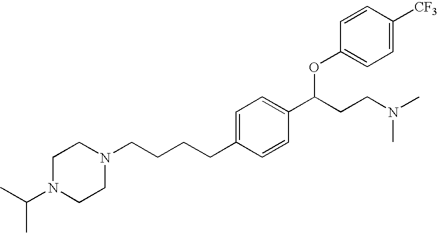

MS (ESI): mass calcd. for C₂₉H₄₂F₃N₃O, 505.33; m/z found, 507.0 [M+H]⁺. ¹H NMR (CDCl₃): 12.46 (bs, 2H), 7.40 (d, J=8.6, 2H), 7.21 (d, J=7.8, 2H), 7.10 (d, J=7.8, 2H), 6.85 (d, J=8.6, 2H), 5.32-5.26 (m, 1H), 3.68-3.40 (m, 9H), 3.26-3.14 (m, 2H), 3.05-2.94 (m, 2H), 2.88-2.74 (m, 6H), 2.64-2.56 (m, 2H), 2.44-2.23 (m, 2H), 1.73-1.59 (m, 4H), 1.33 (d, J=6.3, 6H).

### Example 35

### Dimethyl-[3-[4-(4-morpholin-4-yl-butyl)-phenyl]-3-(4-trifluoromethyl-phenoxy)-propyl]-amine trifluoroacetic acid salt

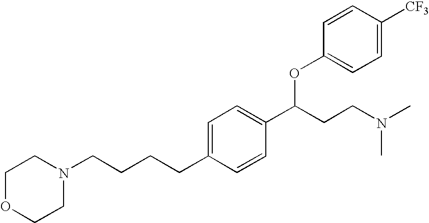

MS (ESI): mass calcd. for C₂₆H₃₅F₃N₂O₂, 464.27; m/z found, 465.5 [M+H]⁺. ¹H NMR (CDCl₃): 12.64-12.29 (m, 2H), 7.43 (d, J=8.6, 2H), 7.22 (d, J=8.0, 2H), 7.12 (d, J=8.0, 2H), 6.86 (d, J=8.6, 2H), 5.32 (dd, J=7.4, 4.4, 1H), 4.03-3.86 (m, 4H), 3.51 (d, J=11.9, 2H), 3.28-3.16 (m, 2H), 3.00-2.93 (m, 2H), 2.90-2.73 (m, 8H), 2.62 (t, J=6.7, 2H), 2.45-2.29 (m, 2H), 1.76-1.60 (m, 4H).

### Example 36

### {3-[4-(1-Isopropyl-piperidin-4-yloxy)-phenyl]-3-phenoxy-propyl}-dimethyl-amine

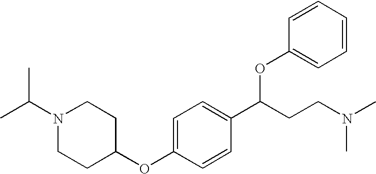

Step A: 1-[4-(1-Isopropyl-piperidin-4-yloxy)-phenyl]-ethanone. To a solution of 1-isopropyl-piperidin-4-ol (2.5 g, 17 mmol) in DMF (34.75 mL) was added slowly NaH (60%; 694 mg, 17.4 mmol). After 30 min at rt, the mixture was treated with 1-(4-fluoro-phenyl)-ethanone (1.5 g, 8.68 mmol). After 20 h at 120° C., the mixture was cooled to rt, diluted with water, and extracted with CH₂Cl₂. The combined organic layers were washed with brine, dried, and concentrated. Purification by FCC gave the desired product (1.52 g, 67%). MS (ESI): mass calcd. for C₁₆H₂₃NO₂, 261.17; m/z found, 262.1 [M+H]⁺. ¹H NMR (CDCl₃): 7.92 (d, J=8.7, 2H), 6.93 (d, J=8.7, 2H), 4.47-4.36 (m, 1H), 2.85-2.74 (m, 3H), 2.55 (s, 3H), 2.46-2.38 (m, 2H), 2.08-2.00 (m, 2H), 1.88-1.80 (m, 2H), 1.07 (s, J=6.5, 6H).

Step B: 1-[4-(1-Isopropyl-piperidin-4-yloxy)-phenyl]-ethanone. The title compound was prepared according to Example 1, Step C, using workup 2. MS (ESI): mass calcd. for C₁₉H₃₀N₂O₂, 318.23; m/z found, 319.2 [M+H]⁺. ¹H NMR (CDCl₃): 7.93 (d, J=8.9, 2H), 6.93 (d, J=8.9 z, 2H), 4.43-4.37 (m, 1H), 3.10 (t, J=7.1, 2H), 2.82-2.72 (m, 5H), 2.45-2.38 (m, 2H), 2.29 (s, 6H), 2.07-2.01 (m, 2H), 1.88-1.80 (m, 2H), 1.06 (d, J=6.6, 6H).

Step C: 3-Dimethylamino-1-[4-(1-isopropyl-piperidin-4-yloxy)-phenyl]-propan-1-ol. The title compound was prepared according to Example 1, Step D. MS (ESI): mass calcd. for C₁₉H₃₂N₂O₂, 320.25; m/z found, 321.2 [M+H]⁺. ¹H NMR (CDCl₃): 7.27 (d, J=8.5, 2H), 6.88 (d, J=8.6, 2H), 4.87-4.84 (m, 1H), 4.30-4.24 (m, 1H), 2.81-2.70 (m, 3H), 2.67-2.61 (m, 1H), 2.48-2.43 (m, 1H), 2.41-2.34 (m, 2H), 2.29 (s, 6H), 2.03-1.96 (m, 2H), 1.85-1.75 (m, 4H), 1.05 (d, J=6.6, 6H).

Step D. The title compound was prepared according to Example 1, Step E. MS (ESI): mass calcd. for C₂₅H₃₆N₂O₂, 396.28; m/z found, 397.2 [M+H]⁺. ¹H NMR (CDCl₃): 7.27-7.23 (m, 2H), 7.18 (t, J=7.9, 2H), 6.87-6.82 (m, 5H), 5.16-5.13 (m, 1H), 4.27-4.21 (m, 1H), 2.80-2.70 (m, 3H), 2.42-2.33 (m, 4H), 2.22 (s, 6H), 2.20-2.10 (m, 1H), 2.02-1.87 (m, 3H), 1.83-1.74 (m, 3H), 1.05 (d, J=6.5, 6H).

### Example 37

### Dimethyl-{3-phenyl-3-[3-(3-piperidin-1-yl-propoxy)-phenoxy]-propyl}-amine

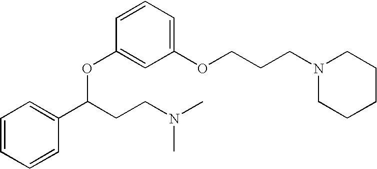

Step A: [3-(3-Methoxy-phenoxy)-3-phenyl-propyl]-dimethyl-amine. The title compound was prepared according to Example 1, Steps C-E.

Step B: 3-(3-Dimethylamino-1-phenyl-propoxy)-phenol. To a solution of [3-(3-methoxy-phenoxy)-3-phenyl-propyl]-dimethyl-amine (500 mg, 1.75 mmol) in CH₂Cl₂ (17.5 mL) was added BBr₃ (0.83 mL, 8.8 mmol). After 3 h at rt, the mixture was diluted with satd. aq. NaHCO₃ and extracted with CH₂Cl₂. The combined organic layers were washed with satd. aq. NaCl, dried, and concentrated. Purification by FCC gave the desired product (65 mg, 14%). MS (ESI): mass calcd. for C₁₇H₂₁NO₂; 271.16; m/z found, 272.1. ¹H NMR (CDCl₃): 7.37-7.29 (m, 5H), 7.28-7.21 (m, 1H), 6.55 (d, J=8.4, 1H), 6.49 (d, J=8.4, 1H), 6.27 (dd, J=8.4, 2.6 Hz, 1H), 4.43 (dd, J=13.0, 3.4, 1H), 2.55-2.48 (m, 1H), 2.45-2.37 (m, 1H), 2.35 (s, 6H), 2.30-2.24 (m, 1H), 2.09-2.01 (m, 1H).

Step C. The title compound was prepared according to Example 1, Step E. MS (ESI): mass calcd. for C₂₅H₃₆N₂O₂, 396.28; m/z found, 397.2 [M+H]⁺. ¹H NMR (CDCl₃): 7.34-7.28 (m, 5H), 7.23-7.19 (m, 1H), 6.54-6.50 (m, 2H), 6.29-6.27 (m, 1H), 4.40-4.4.37 (m, 1H), 3.94-3.90 (m, 2H), 2.49-2.42 (m, 4H), 2.39-2.35 (m, 4H), 2.30 (s, 6H), 2.22-2.19 (m, 1H), 2.03-1.99 (m, 1H), 1.97-1.89 (m, 2H), 1.62-1.54 (m, 6H).

### Example 38

### [4-(3-Dimethylamino-1-phenoxy-propyl)-phenyl]-(4-isopropylpiperazin-1-yl)-methanone

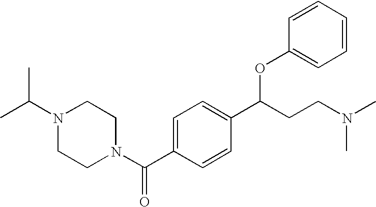

Step A: 1-[4-(4-Isopropyl-piperazine-1-carbonyl)-phenyl]-ethanone. To a 0° C. solution of 4-acetyl-benzoic acid (1.5 g, 9.1 mmol), 1-isopropyl-piperazine dihydrochloride (1.83 g, 9.14 mmol), and N-methylmorpholine (3.0 mL, 27 mmol) in DMF (18.2 mL) was added 1-hydroxybenzotriazole (2.1 g, 15 mmol) and 1-(3-dimethylaminopropyl)-3-ethylcarbodiimide hydrochloride (2.63 g, 13.7 mmol). The reaction mixture was allowed to stir overnight with gradual warming to rt. The mixture was concentrated and the residue was diluted with water, basified to pH ˜9-10 with 1 M NaOH, and extracted with EtOAc. The combined organic layers were dried and concentrated. Purification by FCC gave the desired product (1.99 g, 79%). MS (ESI): mass calcd. for C₁₆H₂₂N₂O₂, 274.17; m/z found, 275.3 [M+H]⁺. ¹H NMR (CDCl₃): 8.08-7.98 (m, 2H), 7.57-7.48 (m, 2H), 3.87-3.77 (m, 2H), 3.46-3.36 (m, 2H), 3.04-2.96 (m, 2H), 2.95-2.86 (m, 2H), 2.83-2.70 (m, 1H), 2.69-2.58 (m, 4H), 1.53-2.43 (m, 1H), 1.13-1.03 (m, 4H).

Step B: 3-Dimethylamino-1-[4-(4-isopropyl-piperazine-1-carbonyl)phenyl]-propan-1-one. The title compound was prepared according to Example 1, Step C, using workup 1. MS (ESI): mass calcd. for C₁₉H₂₉N₃O₂, 331.23; m/z found, 332.2 [M+H]⁺. ¹H NMR (DMSO): 8.09 (d, J=8.2, 2H), 7.68 (d, J=8.2, 2H), 4.65-4.54 (m, 1H), 3.94-3.22 (m, 12H), 3.17-3.05 (m, 2H), 2.80 (d, J=4.9, 4H), 1.30 (d, J=6.6, 6H).

Step C: [4-(3-Dimethylamino-1-hydroxy-propyl)-phenyl]-(4-isopropylpiperazin-1-yl)-methanone. The title compound was prepared according to Example 1, Step D. MS (ESI): mass calcd. for C₁₉H₃₁N₃O₂, 333.24; m/z found, 334.2 [M+H]⁺. ¹H NMR (CDCl₃): 7.43 (m, 4H), 4.95 (dd, J=3.4, 8.1, 1H), 3.85-3.68 (m, 2H), 3.50-3.37 (m, 2H), 2.77-2.54 (m, 4H), 2.51-2.41 (m, 3H), 2.31 (s, 6H), 1.88-1.72 (m, 2H), 1.05 (d, J=6.5, 6H).

Step D. The title compound was prepared according to Example 1, Step E. MS (ESI): mass calcd. for C₂₅H₃₅N₃O₂, 409.27; m/z found, 410.2 [M+H]⁺. ¹H NMR (CDCl₃): 7.42-7.35 (m, 4H), 7.21-7.15 (m, 2H), 6.90-6.82 (m, 3H), 5.27-5.22 (m, 1H), 3.84-3.74 (m, 2H), 3.47-3.38 (m, 2H), 2.72 (heptet, J=6.5, 1H), 2.63-2.37 (m, 6H), 2.23 (s, 6H), 2.20-2.10 (m, 1H), 1.98-1.87 (m, 1H), 1.04 (d, J=6.5, 6H).

The compounds in Examples 39-40 were prepared as in Example 38.

### Example 39

### {4-[3-Dimethylamino-1-(4-trifluoromethyl-phenoxy)-propyl]-phenyl}-(4-isopropyl-piperazin-1-yl)-methanone

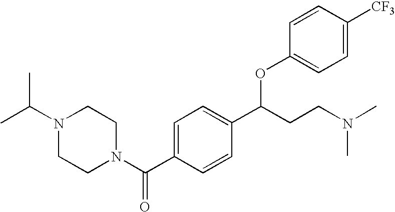

MS (ESI): mass calcd. for C₂₆H₃₄F₃N₃O₂, 477.26; m/z found, 478.2 [M+H]⁺. ¹H NMR (CDCl₃): 7.44 (d, J=8.7, 2H), 7.39 (s, 4H), 6.90 (d, J=8.7, 2H), 5.34-5.29 (m, 1H), 3.82-3.70 (m, 2H), 3.46-3.35 (m, 2H), 2.73 (heptet, J=6.5, 1H), 2.64-2.31 (m, 5H), 2.23 (s, 6H), 2.20-2.10 (m, 1H), 1.99-1.88 (m, 2H), 1.04 (d, J=6.5, 6H).

### Example 40

### {4-[1-(3,4-Dichloro-phenoxy)-3-dimethylamino-propyl]-phenyl}-(4-isopropyl-piperazin-1-yl)-methanone

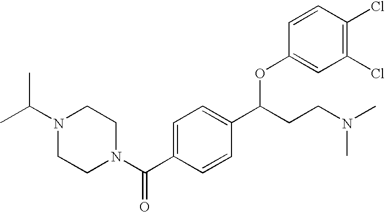

MS (ESI): mass calcd. for C₂₅H₃₃Cl₂N₃O₂, 477.19; m/z found, 478.4, 480.5 [M+H]⁺. ¹H NMR (CDCl₃): 7.40-7.33 (m, 4H), 7.21 (d, J=8.9, 1H), 6.96 (d, J=2.9, 1H), 6.68 (dd, J=8.9, 2.9, 1H), 5.23 (dd, J=8.0, 5.0, 1H), 3.84-3.70 (m, 2H), 3.49-3.35 (m, 2H), 2.76-2.67 (m, 1H), 2.62-2.53 (m, 1H), 2.49-2.30 (m, 4H), 2.25 (s, 6H), 2.19-2.07 (m, 1H), 2.03-1.88 (m, 2H), 1.04 (d, J=6.5, 6H).

### Example 41

### Dimethyl-{3-phenyl-3-[4-(3-piperidin-1-yl-propoxy)-phenoxy]-propyl}-amine

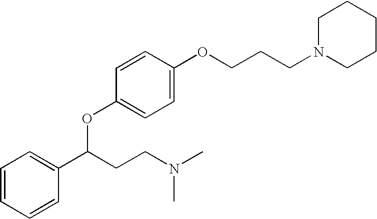

Step A: 1-[3-(4-Benzyloxy-phenoxy)-propyl]-piperidine. The title compound was prepared according to Example 1, Step E. MS (ESI): mass calcd. for C₂₁H₂₇NO₂, 325.20; m/z found, 326.2 [M+H]⁺.

Step B: 4-(3-Piperidin-1-yl-propoxy)-phenol. N₂ gas was bubbled through a solution of 1-[3-(4-benzyloxy-phenoxy)-propyl]-piperidine (290 mg, 0.891 mmol) in EtOH (2.2 mL) and CH₂Cl₂ (0.4 mL) before 10% Pd/C (101.5 mg) was added. The solution was subjected to degassing before a H₂ balloon was attached to the reaction flask. The reaction was stirred under H₂ at rt for 3 days. The reaction mixture was filtered and the filtrate was concentrated. Purification by FCC gave the desired product (179 mg, 85%). MS (ESI): mass calcd. for C₁₄H₂₁NO₂, 235.16; m/z found, 236.2 [M+H]⁺. ¹H NMR (CDCl₃): 6.66 (s, 4H), 3.90 (t, J=6.3, 2H), 2.56-2.45 (m, 5H), 1.98-1.92 (m, 2H), 1.65 (quintet, J=5.7, 4H), 1.51-1.39 (m, 3H).

Step C. The title compound was prepared according to Example 1, Step E, using 4-(3-piperidin-1-yl-propoxy)-phenol and 3-dimethylamino-1-phenylpropan-1-ol. This compound was isolated by preparative reverse phase HPLC to give the trifluoroacetic acid salt. The salt was converted to its corresponding free base using OH⁻ Dowex ion exchange resin. MS (ESI): mass calcd. for C₂₅H₃₆N₂O₂, 396.28; m/z found, 397.3 [M+H]⁺. ¹H NMR (CDCl₃): 7.44-7.33 (m, 4H), 7.31-7.27 (m, 1H), 6.84-6.80 (m, 2H), 6.80-6.75 (m, 2H), 5.31-5.27 (m, 1H), 4.01-3.97 (m, 2H), 3.60-3.55 (m, 2H), 3.45-3.39 (m, 1H), 3.37-3.32 (m, 1H), 3.29-3.24 (m, 2H), 2.97-2.91 (m, 8H), 2.42-2.34 (m, 1H), 2.31-2.22 (m, 1H), 2.20-2.13 (m, 2H), 2.00-1.92 (m, 2H), 1.88-1.71 (m, 3H), 1.58-1.47 (m, 1H).

The compounds in Examples 42-45 were prepared as in Example 41.

### Example 42

### Dimethyl-[3-[4-(3-piperidin-1-yl-propoxy)-phenoxy]-3-(4-trifluoromethyl-phenyl)-propyl]-amine

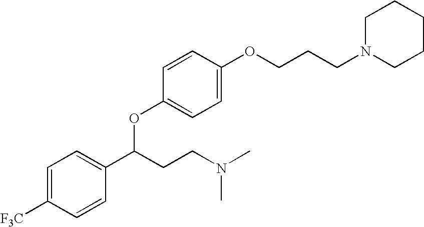

MS (ESI): mass calcd. for C₂₆H₃₅F₃N₂O₂, 464.27; m/z found, 465.2 [M+H]⁺. ¹H NMR (CDCl₃): 7.58 (d, J=8.2, 2H), 7.47 (d, J=8.1, 2H), 6.75-6.71 (m, 4H), 5.19-5.16 (m, 1H), 3.89 (t, J=6.4, 2H), 2.47-2.34 (m, 8H), 2.22 (s, 6H), 2.17-2.09 (m, 1H), 1.94-1.88 (m, 3H), 1.59-1.54 (m, 4H), 1.46-1.38 (m, 2H).

### Example 43

### {3-(4-Chloro-phenyl)-3-[4-(3-piperidin-1-yl-propoxy)-phenoxy]-propyl}-dimethyl-amine

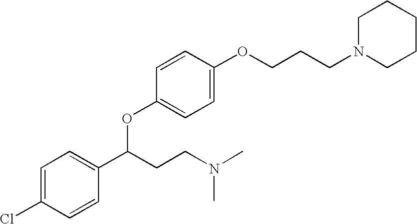

¹H NMR (CDCl₃): 7.32-7.26 (m, 4H), 6.76-6.68 (m, 4H), 5.08 (dd, J=8.1, 5.1, 1H), 3.89 (t, J=6.4, 2H), 2.49-2.34 (m, 8H), 2.22 (s, 6H), 2.21-2.08 (m, 2H), 1.97-1.86 (m, 2H), 1.62-1.55 (m, 4H), 1.49-1.38 (m, 2H).

### Example 44

### {3-[4-(1-Isopropyl-piperidin-4-yloxy)-phenoxy]-3-phenyl-propyl}-dimethyl-amine

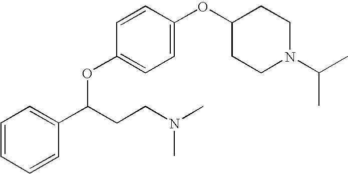

MS (ESI): mass calcd. for C₂₅H₃₆N₂O₂, 396.28; m/z found, 397.3 [M+H]⁺. ¹H NMR (CDCl₃): 7.39-7.29 (m, 4H), 7.26-7.22 (m, 1H), 6.77-6.70 (m, 4H), 5.09 (dd, J=8.2, 4.9, 1H), 4.13-4.05 (m, 1H), 2.79-2.66 (m, 3H), 2.41 (t, J=7.2, 2H), 2.31 (t, J=11.5, 2H), 2.22 (s, 6H), 2.18-2.08 (m, 1H), 1.97-1.88 (m, 3H), 1.81-1.68 (m, 2H), 1.03 (d, J=6.6, 6H).

### Example 45

### [3-[4-(1-Isopropyl-piperidin-4-yloxy)-phenoxy]-3-(4-trifluoromethylphenyl)-propyl]-dimethyl-amine

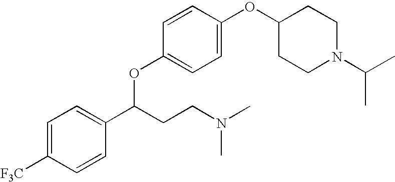

MS (ESI): mass calcd. for C₂₆H₃₅F₃N₂O₂, 464.27; m/z found, 465.3 [M+H]⁺. ¹H NMR (CDCl₃): 7.58 (d, J=8.2, 2H), 7.47 (d, J=8.1, 2H), 6.73 (s, 4H), 5.19-5.16 (m, 1H), 4.12-4.08 (m, 1H), 2.77-2.69 (m, 3H), 2.47-2.30 (m, 4H), 2.22 (s, 6H), 2.17-2.09 (m, 1H), 1.95-1.87 (m, 3H), 1.77-1.70 (m, 2H), 1.03 (d, J=6.5, 6H).

### Example 46

### {4-[3-Dimethylamino-1-(4-trifluoromethyl-phenyl)-propoxy]-phenyl}-(4-isopropyl-piperazin-1-yl)-methanone

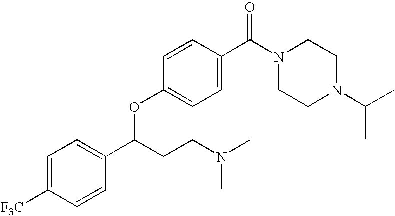

Step A: (4-Benzyloxy-phenyl)-(4-isopropyl-piperazin-1-yl)-methanone. The title compound was prepared according to Example 38, Step A. MS (ESI): mass calcd. for C₂₁H₂₆N₂O₂, 338.20; m/z found, 339.2 [M+H]⁺.

Step B: ((4-Hydroxy-phenyl)-(4-isopropyl-piperazin-1-yl)-methanone. The title compound was prepared according to Example 41, Step B. MS (ESI): mass calcd. for C₁₄H₂₀N₂O₂, 248.15; m/z found, 249.2 [M+H]⁺.

Step C. The title compound was prepared according to Example 1, Step E. MS (ESI): mass calcd. for C₂₆H₃₄F₃N₃O₂, 477.26; m/z found, 478.2 [M+H]⁺. ¹H NMR (CDCl₃): 7.59 (d, J=8.2, 2H), 7.48 (d, J=8.1, 2H), 7.29-7.27 (m, 2H), 6.84-6.82 (m, 2H), 5.35-5.32 (m, 1H), 3.35-3.85 (m, 4H), 2.72-2.67 (m, 1H), 2.48-2.42 (m, 4H), 2.38-2.33 (m, 1H), 2.22 (s, 6H), 2.20-2.13 (m, 2H), 1.97-1.90 (m, 1H), 1.03 (d, J=6.5, 6H).

### Example 47

### {3-(3,4-Dichloro-phenoxy)-3-[4-(4-isopropyl-piperazin-1-ylmethyl)phenyl]-propyl}-dimethyl-amine trifluoroacetic acid salt

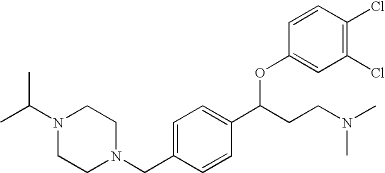

Step A: [3-(3,4-Dichloro-phenoxy)-3-(4-iodo-phenyl)-propyl]-dimethyl-amine. The title compound was prepared in an analogous fashion to Example 1, Steps C-E. MS (ESI): mass calcd. for C₁₇H₁₈Cl₂NO, 448.98; m/z found, 450.2 [M+H]⁺.

Step B: {4-[1-(3,4-Dichloro-phenoxy)-3-dimethylamino-propyl]-phenyl}-(4-isopropyl-piperazin-1-yl)-methanone. To a solution of 3-dimethylamino-1-(4-iodo-phenyl)-propan-1-ol (289 mg, 0.640 mmol) in THF (4 mL) was added DBU (293 mg, 1.93 mmol), Pd(OAc)₂ (29 mg, 0.128 mmol), a solution of 1-isopropylpiperazine (247 mg, 1.93 mmol) in THF (2 mL), and Mo(CO)₆ (169 mg, 0.640 mmol). The mixture was heated in a microwave reactor for 15 min at 100° C., cooled to rt, and filtered through a pad of diatomaceous earth. The filtrate was concentrated and the residue purified by acidic reverse phase HPLC to give the desired product (225 mg, 35%) as an orange/brown oil. MS (ESI): mass calcd. for C₂₅H₃₃Cl₂N₃O₂, 477.19; m/z found, 478.8 [M+H]⁺. ¹H NMR (MeOD): 7.54-7.49 (m, 4H), 7.28 (d, J=8.9, 1H), 7.07-7.06 (m, 1H), 6.83 (dd, J=8.9, 2.9, 1H), 5.49-5.46 (m, 1H), 3.56-3.50 (m, 2H), 3.42-3.27 (m, 7H), 2.89 (s, 6H), 2.43-2.33 (m, 2H), 2.31-2.22 (m, 2H), 1.34 (d, J=6.6, 6H).

Step C. To a solution of {4-[1-(3,4-dichloro-phenoxy)-3-dimethylamino-propyl]-phenyl}-(4-isopropyl-piperazin-1-yl)-methanone (37 mg, 0.077 mmol) in THF (0.4 mL) was added BH₃.THF (1.0 M in THF; 0.315 mL, 0.315 mmol). The mixture was heated at reflux for 5 h and additional BH₃-THF was added (0.315 mL, 0.315 mmol). The mixture was the heated at reflux for 18 h, cooled to rt, and concentrated. The residue was treated with satd. aq. NaHCO₃ and extracted with EtOAc (3×). The combined organic layers were dried and concentrated. The residue was purified by FCC followed by preparative reverse phase HPLC to give the desired product (9 mg, 24%). MS (ESI): mass calcd. for C₂₅H₃₅Cl₂N₃O, 463.22; m/z found, 464.5.

The compounds in Examples 48-56 were prepared as in Example 47.

### Example 48

### (4-Butyl-piperazin-1-yl)-{4-[3-dimethylamino-1-(4-trifluoromethylphenoxy)-propyl]-phenyl}-methanone trifluoroacetic acid salt

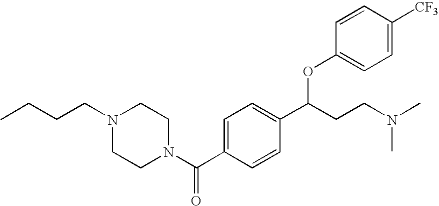

MS (ESI): mass calcd. for C₂₇H₃₆F₃N₃O₂, 491.28; m/z found, 492.5 [M+H]⁺. ¹H NMR (MeOD): 7.55-7.45 (m, 6H), 7.02 (d, J=8.7, 2H), 5.58-5.55 (m, 1H), 3.43-3.30 (m, 5H), 3.28-3.27 (m, 2H), 3.25-3.14 (m, 2H), 3.13-3.07 (m, 1H), 2.89 (s, 6H), 2.47-2.26 (m, 2H), 2.11-2.08 (m, 1H), 1.92-1.88 (m, 1H), 1.75-1.65 (m, 2H), 1.50-1.13 (m, 4H), 0.97-0.94 (m, 1H).

### Example 49

### [3-[4-(4-Butyl-piperazin-1-ylmethyl)-phenyl]-3-(4-trifluoromethylphenoxy)-propyl]-dimethyl-amine trifluoroacetic acid salt

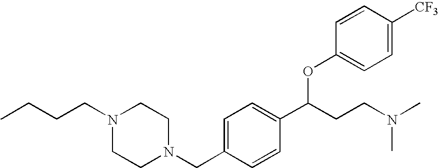

MS (ESI): mass calcd. for C₂₇H₃₈F₃N₃O, 477.30; m/z found, 478.6 [M+H]⁺. ¹H NMR (MeOD): 7.5 (d, J=8.7, 2H), 7.44 (d, J=8.2, 2H), 7.40 (d, J=8.2, 2H), 7.04 (d, J=8.7, 2H) 5.53-5.50 (m, 1H), 3.66-3.62 (m, 2H), 3.46-3.40 (m, 1H), 3.37-3.33 (m, 4H), 3.10-3.06 (m, 3H), 2.94 (s, 6H), 2.47-2.27 (m, 3H), 1.72-1.65 (m, 2H), 1.45-1.36 (m, 2H), 1.34-1.27 (m, 3H), 1.00 (t, J=7.3, 3H).

### Example 50

### (4-Cyclopentyl-piperazin-1-yl)-{4-[3-dimethylamino-1-(4-trifluoromethyl-phenoxy)-propyl]-phenyl}-methanone trifluoroacetic acid salt

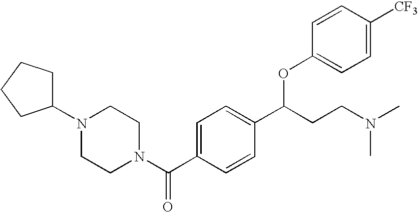

MS (ESI): mass calcd. for C₂₈H₃₆F₃N₃O₂, 503.28; m/z found, 504.6 [M+H]⁺. ¹H NMR (MeOD): 7.56-7.54 (m, 2H), 7.50-7.46 (m, 4H), 7.02 (d, J=8.6, 2H), 5.58-5.55 (m, 1H), 3.64-3.45 (m, 3H), 3.44-3.30 (m, 4H), 3.29-3.27 (m, 2H), 3.26-3.04 (m, 2H), 2.90 (s, 6H), 2.46-2.26 (m, 2H), 2.17-2.06 (m, 2H), 1.81-1.64 (m, 6H).

### Example 51

### [3-[4-(4-Cyclopentyl-piperazin-1-ylmethyl)-phenyl]-3-(4-trifluoromethyl-phenoxy)-propyl]-dimethyl-amine trifluoroacetic acid salt

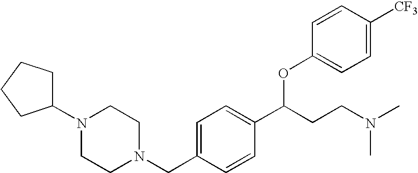

MS (ESI): mass calcd. for C₂₈H₃₈F₃N₃O, 489.30; m/z found, 490.5 [M+H]⁺. ¹H NMR (CDCl₃): 13.1 (bs, 1H), 7.47-7.43 (m, 4H), 7.39-7.38 (m, 2H), 6.85 (d, J=8.8, 2H), 5.42 (t, J=5.8, 1H), 4.14-4.08 (m, 2H), 3.67-3.43 (m, 8H), 3.37-3.30 (m, 1H), 3.28-3.20 (m, 1H), 3.19-3.12 (m, 1H), 2.84-2.82 (m, 6H), 2.40-2.34 (m, 2H), 2.07-1.80 (m, 8H).

### Example 52

### (4-sec-Butyl-piperazin-1-yl)-{4-[3-dimethylamino-1-(4-trifluoromethyl-phenoxy)-propyl]-phenyl}-methanone trifluoroacetic acid salt

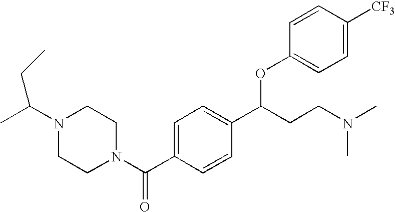

MS (ESI): mass calcd. for C₂₇H₃₆F₃N₃O₂, 491.28; m/z found, 492.5 [M+H]⁺. ¹H NMR (MeOD): 7.52-7.35 (m, 6H), 6.95 (d, J=8.7, 2H), 5.49 (dd, J=8.7, 3.7, 1H), 3.41-3.03 (m, 11H), 2.83 (s, 6H), 2.39-2.18 (m, 2H), 1.86-1.74 (m, 1H), 1.54-1.42 (m, 1H), 1.25 (d, J=6.7, 3H), 0.93 (t, J=7.4, 3H).

### Example 53

### {4-[3-Dimethylamino-1-(4-trifluoromethyl-phenoxy)-propyl]-phenyl}-[4-(1-ethyl-propyl)-piperazin-1-yl]-methanone trifluoroacetic acid salt

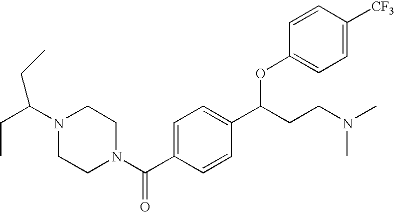

MS (ESI): mass calcd. for C₂₈H₃₈F₃N₃O₂, 505.29; m/z found, 506.5 [M+H]⁺. ¹H NMR (MeOD): 7.57-7.43 (m, 6H), 7.02 (d, J=8.7, 2H), 5.57 (dd, J=8.7, 3.8, 1H), 4.12-3.13 (m, 10H), 3.13-3.06 (m, 1H), 2.90 (s, 6H), 2.47-2.35 (m, 1H), 2.36-2.24 (m, 1H), 1.92-1.79 (m, 2H), 1.77-1.64 (m, 2H), 1.02 (t, J=7.5, 6H).

### Example 54

### [3-{4-[4-(1-Ethyl-propyl)-piperazin-1-ylmethyl]-phenyl}-3-(4-trifluoromethyl-phenoxy)-propyl]-dimethyl-amine trifluoroacetic acid salt

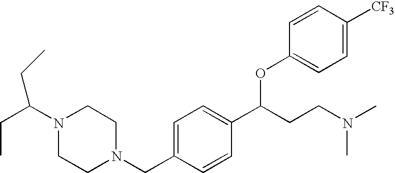

MS (ESI): mass calcd. for C₂₈H₄₀F₃N₃O, 491.31; m/z found, 492.5 [M+H]⁺. ¹H NMR (acetone-d₆): 7.56-7.51 (m, 6H), 7.10-7.07 (m, 2H), 5.70-5.67 (m, 1H), 4.20-4.18 (m, 2H), 3.57-3.48 (m, 2H), 3.47-3.34 (m, 6H), 3.08-2.99 (m, 1H), 2.98-2.84 (m, 6H), 2.55-2.40 (m, 2H), 2.07-2.02 (m, 2H), 1.90-1.82 (m, 2H), 1.71-1.62 (m, 2H), 1.04-0.94 (m, 6H).

### Example 55

### [3-[4-(4-Isopropyl-piperazin-1-ylmethyl)-phenyl]-3-(4-trifluoromethyl-phenoxy)-propyl]-dimethyl-amine trifluoroacetic acid salt

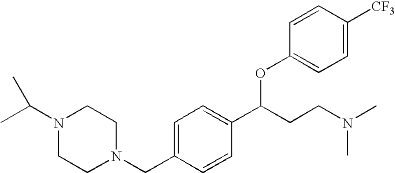

MS (ESI): mass calcd. for C₂₆H₃₆F₃N₃O, 463.28; m/z found, 464.5 [M+H]⁺.

### Example 56

### {3-[4-(4-Isopropyl-piperazin-1-ylmethyl)-phenyl]-3-phenoxy-propyl}-dimethyl-amine

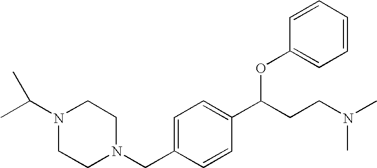

MS (ESI): mass calcd. for C₂₅H₃₇N₃O, 395.29; m/z found, 396.5 [M+H]⁺. ¹H NMR (CDCl₃): 7.23 (d, J=8.1, 2H), 7.19 (d, J=8.0, 2H), 7.10 (dd, J=8.6, 7.42 Hz, 2H), 6.81-6.76 (m, 3H), 5.13 (dd, J=8.2, 4.8, 1H), 3.42 (s, 2H), 2.71-2.42 (m, 10H), 2.39 (t, J=7.2, 2H), 2.19 (s, 1H), 2.14-2.05 (m, 1H), 1.93-1.85 (m, 1H), 1.81-1.47 (m, 4H), 1.02 (d, J=6.3, 6H).

### Example 57

### Dimethyl-{3-phenyl-3-[4-(4-piperidin-1-yl-but-1-ynyl)-phenoxy]-propyl}-amine

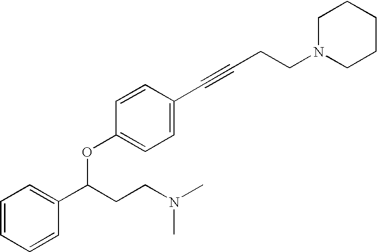

Step A: [3-(4-Bromo-phenoxy)-3-phenyl-propyl]-dimethyl-amine. The title compound was prepared in an analogous fashion to Example 1, Steps C-E.

Step B. The title compound was prepared in an analogous fashion to Example 20, Step D. MS (ESI): mass calcd. for C₂₆H₃₄N₂O, 390.27; m/z found, 391.3 [M+H]⁺. ¹H NMR (CDCl₃): 7.35-7.29 (m, 5H), 7.19 (d, J=8.6, 2H), 6.74 (d, J=8.0, 2H), 5.21-5.19 (m, 1H), 2.62-2.59 (m, 2H), 2.55-2.52 (m, 2H), 2.44-2.37 (m, 6H), 2.22 (s, 6H), 2.20-2.13 (m, 1H), 1.98-1.91 (m, 1H), 1.60-1.56 (m, 4H), 1.43-1.42 (m, 2H).

The compounds in Examples 58-64 were prepared as in Example 59.

### Example 58

### {3-(4-Chloro-phenyl)-3-[4-(4-thiomorpholin-4-yl-but-1-ynyl)phenoxy]-propyl}-dimethyl-amine trifluoroacetic acid salt

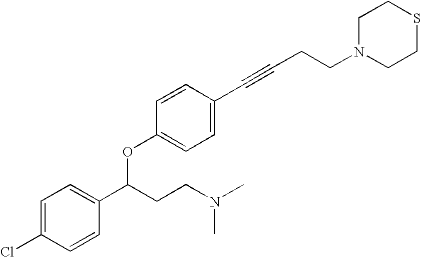

MS (ESI): mass calcd. for C₂₅H₃₁ClN₂OS, 442.18; m/z found, 443.1 [M+H]⁺. ¹H NMR (MeOD): 7.35-7.29 (m, 4H), 7.14 (d, J=8.9, 2H), 6.74 (d, J=8.9, 2H), 5.32 (dd, J=8.5, 4.0, 1H), 3.29-3.27 (m, 2H), 3.22-3.13 (m, 2H), 2.85-2.75 (m, 9H), 2.67-2.60 (m, 5H), 2.52 (t, J=7.4, 2H), 2.32-2.13 (m, 2H).

### Example 59

### {3-(4-Chloro-phenyl)-3-[4-(4-morpholin-4-yl-but-1-ynyl)-phenoxy]-propyl}-dimethyl-amine trifluoroacetic acid salt

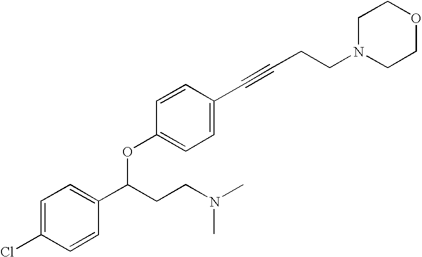

MS (ESI): mass calcd. for C₂₅H₃₁ClN₂O₂, 426.21; m/z found, 427.2 [M+H]⁺. ¹H NMR (MeOD): 7.34-7.30 (m, 4H), 7.14 (d, J=8.9, 2H), 6.74 (d, J=8.9, 2H), 5.32-5.30 (m, 1H), 3.63 (t, J=4.6, 4H), 2.74 (s, 4H), 2.58-2.51 (m, 4H), 2.31-2.22 (m, 1H), 2.20-2.12 (m, 1H), 1.97-1.96 (m, 8H).

### Example 60

### Dimethyl-[3-[4-(4-piperidin-1-yl-but-1-ynyl)-phenoxy]-3-(4-trifluoromethyl-phenyl)-propyl]-amine

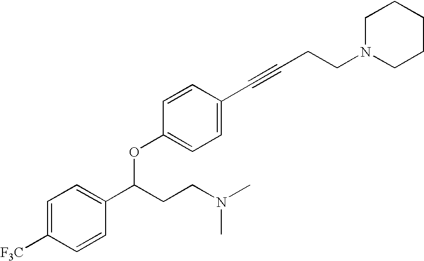

MS (ESI): mass calcd. for C₂₇H₃₃F₃N₂O, 458.25; m/z found, 459.2 [M+H]⁺. ¹H NMR (CDCl₃): 7.58 (d, J=8.2, 2H), 7.45 (d, J=8.1, 2H), 7.21 (d, J=8.8, 2H), 6.72 (d, J=8.8, 1H), 5.29 (dd, J=8.0, 5.0, 1H), 2.63-2.58 (m, 2H), 2.58-2.52 (m, 2H), 2.57-2.49 (m, 1H), 2.47-2.41 (m, 4H), 2.38-2.32 (m, 2H), 2.22 (s, 6H), 2.19-2.11 (m, 1H), 1.96-1.89 (m, 1H), 1.61-1.55 (m, 4H), 1.47-1.40 (m, 2H).

### Example 61

### Dimethyl-{3-[4-(4-morpholin-4-yl-but-1-ynyl)-phenoxy]-3-phenyl-propyl}-amine

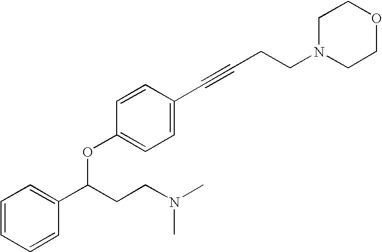

MS (ESI): mass calcd. for C₂₅H₃₂N₂O₂, 392.25; m/z found, 393.2 [M+H]⁺. ¹H NMR (CDCl₃): 7.35-7.28 (m, 4H), 7.26-7.22 (m, 1H), 7.19 (d, J=8.8 Hz, 2H), 6.75 (d, J=8.8, 2H), 5.21 (dd, J=8.1, 5.0, 1H), 2.65-2.59 (m, 2H), 2.58-2.48 (m, 7H), 2.45-2.34 (m, 3H), 2.22 (s, 6H), 2.18-2.11 (m, 1H), 1.99-1.91 (m, 1H), 1.79-1.66 (m, 2H).

### Example 62

### [3-{4-[4-(4-Isopropyl-piperazin-1-yl)-but-1-ynyl]-phenoxy}-3-(4-trifluoromethyl-phenyl)-propyl]-dimethyl-amine

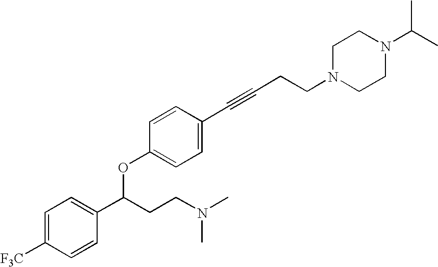

MS (ESI): mass calcd. for C₂₉H₃₈F₃N₃O, 501.30; m/z found, 502.3 [M+H]⁺. ¹H NMR (CDCl₃): 7.58 (d, J=8.2, 2H), 7.45 (d, J=8.1, 2H), 7.20 (d, J=8.5, 2H), 6.72 (d, J=8.6, 2H), 5.30-5.28 (m, 1H), 2.66-2.63 (m, 4H), 2.60-2.51 (m, 9H), 2.45-2.40 (m, 1H), 2.38-2.32 (m, 1H), 2.22 (s, 6H), 2.18-2.11 (m, 1H), 1.95-1.89 (m, 1H), 1.05 (d, J=6.5, 6H).

### Example 63

### Dimethyl-{3-phenyl-3-[4-(4-thiomorpholin-4-yl-but-1-ynyl)phenoxy]-propyl}-amine trifluoroacetic acid salt

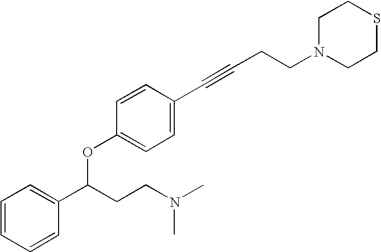

MS (ESI): mass calcd. for C₂₅H₃₂N₂OS, 408.22; m/z found, 409.2 [M+H]⁺. ¹H NMR (MeOD): 7.41-7.29 (m, 4H), 7.29-7.23 (m, 1H), 7.21 (d, J=8.8 Hz, 2H), 6.80 (d, J=8.9, 2H), 5.37 (dd, J=8.5, 4.0, 1H), 4.05-3.58 (m, 2H), 3.42-3.29 (m, 6H), 3.04-2.71 (m, 12H), 2.41-2.31 (m, 1H), 2.28-2.18 (m, 1H).

### Example 64

### Dimethyl-[3-[4-(4-morpholin-4-yl-but-1-ynyl)-phenoxy]-3-(4-trifluoromethyl-phenyl)-propyl]-amine trifluoroacetic acid salt

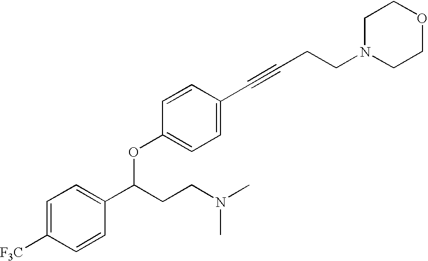

MS (ESI): mass calcd. for C₂₆H₃₁F₃N₂O₂, 460.23; m/z found, 461.2 [M+H]⁺. ¹H NMR (MeOD): 7.65-7.60 (m, 2H), 7.58-7.51 (m, 2H), 7.24-7.19 (m, 2H), 6.83-6.77 (m, 2H), 5.50-5.46 (m, 1H), 4.07-3.92 (m, 2H), 3.79-3.65 (m, 2H), 3.56-3.45 (m, 2H), 3.39-3.28 (m, 4H), 3.22-3.09 (m, 2H), 2.92-2.82 (m, 8H), 2.41-2.21 (m, 2H).

### Example 65

### Dimethyl-{4-phenyl-3-[4-(3-piperidin-1-yl-propoxy)-phenyl]-butyl}-amine

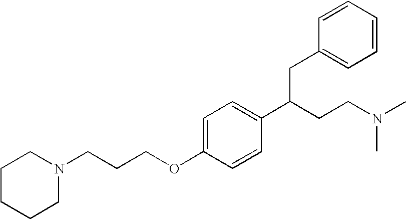

Step A. 4-Dimethylamino-1-phenyl-2-[4-(3-piperidin-1-yl-propoxy)phenyl]-butan-2-ol trifluoroacetic acid salt. To a −78° C. solution of 3-dimethylamino-1-[4-(3-piperidin-1-yl-propoxy)-phenyl]-propan-1-one (431 mg, 1.35 mmol) in THF (13.5 mL) was added benzylmagnesium bromide (1.0 M in Et₂O; 1.62 mL, 1.62 mmol). After 3 h at −78° C. and 18 h at rt, the mixture was diluted with satd. aq. NH₄Cl, EtOAc, and satd. aq. NaHCO₃ until basic. The mixture was extracted with EtOAc and the combined organic layers were dried and concentrated. The residue was purified by FCC and then by reverse phase HPLC to give the desired product (175 mg, 20%) as a TFA salt. MS (ESI): mass calcd. for C₂₆H₃₈N₂O₂, 410.29; m/z found, 411.5 [M+H]⁺.

Step B. To a solution of 4-dimethylamino-1-phenyl-2-[4-(3-piperidin-1-yl-propoxy)-phenyl]-butan-2-ol (152 mg, 0.290 mmol) in TFA (1 mL) at 0° C. was added H₂SO₄ (0.050 mL, 0.928 mmol). After 30 min at 0° C., triethylsilane (0.148 mL, 0.928 mmol) was added. After 3 h at rt, the mixture was diluted with satd. aq. NaHCO₃ and extracted with EtOAc. The combined organic layers were concentrated and purified by FCC to give the desired product (35 mg, 30%). MS (ESI): mass calcd. for C₂₆H₃₈N₂O, 394.30; m/z found, 395.5 [M+H]⁺. ¹H NMR (CDCl₃): 7.21-7.16 (m, 2H), 7.15-7.09 (m, 1H), 7.03-6.98 (m, 4H), 6.81-6.76 (m, 2H), 3.97 (t, J=6.4, 2H), 2.86-2.76 (m, 3H), 2.50-2.35 (m, 6H), 2.15-2.06 (m, 7H), 2.05-1.92 (m, 3H), 1.89-1.80 (m, 1H), 1.76-1.65 (m, 1H), 1.63-1.56 (m, 4H), 1.48-1.40 (m, 2H).

### Example 66

### N³,N³-Dimethyl-N¹-phenyl-1-[4-(3-piperidin-1-yl-propoxy)-phenyl]-propane-1,3-diamine trifluoroacetic acid salt

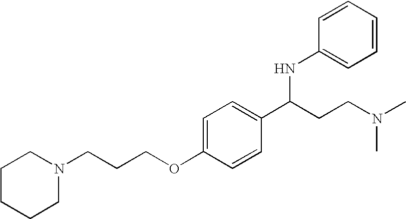

A solution of 3-dimethylamino-1-[4-(3-piperidin-1-yl-propoxy)-phenyl]-propan-1-one (316 mg, 0.99 mmol) and aniline (0.099 mL, 1.1 mmol) in Ti(OiPr)₄ (0.892 mL, 3.0 mmol) was stirred at rt overnight. MeOH (5 mL) and NaBH₄ (113 mg, 3.0 mmol) were then added and the mixture was stirred at rt for 20 min. The mixture was diluted with water and satd. aq. NaHCO₃ and extracted with CH₂Cl₂ (3×). The combined organic layers were dried and concentrated. Purification by reverse phase HPLC gave the desired product (5.7 mg, 1%) as a TFA salt. MS (ESI): mass calcd. for C₂₅H₃₇N₃O, 395.29; m/z found, 396.5 [M+H]⁺. ¹H NMR (CDCl₃): 12.31 (bs, 1H), 7.23 (d, J=8.7, 2H), 7.10 (dd, J=7.4, 8.4, 2H), 6.81 (d, J=8.7, 2H), 6.68 (t, J=7.4, 1H), 6.54 (d, J=7.7, 2H), 4.30-4.28 (m, 1H), 4.01 (t, J=5.5, 2H), 3.67-3.64 (d, J=11.5, 2H), 3.20-3.17 (m, 2H), 2.91 (ddd, J=4.4, 12.1, 12.1, 1H), 2.79-2.75 (m, 1H), 2.71 (ddd, J=5.3, 11.9, 11.9, 1H), 2.66-2.59 (m, 2H), 2.56 (s, 3H), 2.54 (s, 3H), 2.36-2.17 (m, 4H), 2.01-1.98 (m, 2H), 1.91-1.86 (m, 2H), 1.47-1.34 (m, 2H).

### Example 67

### Dimethyl-{3-phenylsulfanyl-3-[4-(3-piperidin-1-yl-propoxy)phenyl]-propyl}-amine trifluoroacetic acid salt

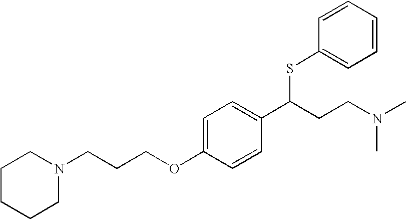

To a 0° C. solution of 3-dimethylamino-1-[4-(3-piperidin-1-yl-propoxy)phenyl]-propan-1-ol (100 mg, 0.312 mmol) and Et₃N (0.065 mL, 0.468 mmol) in CH₂Cl₂ (1.56 mL) was added MsCl (0.026 mL, 0.343 mmol) over 10 min. After 15 min, a solution of thiophenol (0.064 mL, 0.624 mmol) in CH₂Cl₂ (1.56 mL) was added. The reaction mixture was allowed to warm to rt and was stirred for 2 h. The mixture was diluted with satd. aq. NaHCO₃ and extracted with CH₂Cl₂. The combined organic layers were washed with satd. aq. NaCl, dried, concentrated, and purified by reverse phase HPLC to give the desired product (12.1 mg, 4%). MS (ESI): mass calcd. for C₂₅H₃₆N₂OS, 412.25; m/z found, 413.5 [M+H]⁺. ¹H NMR (MeOD): 7.28-7.18 (m, 7H), 6.84-6.82 (m, 2H), 4.26-4.22 (m, 1H), 4.12-4.02 (m, 2H), 3.68-3.48 (m, 2H), 3.29-3.24 (m, 4H), 2.99-2.93 (m, 2H), 2.80 (s, 6H), 2.31-2.15 (m, 4H), 2.15-1.67 (m, 6H).

### Example 68

### 3-Phenoxy-3-[4-(3-piperidin-1-yl-propoxy)-phenyl]-propylamine trifluoroacetic acid salt

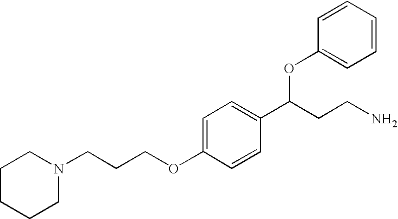

Step A: 1-[4-(3-Piperidin-1-yl-propoxy)-phenyl]-but-3-en-1-ol. To a −78° C. solution of 3-dimethylamino-1-[4-(3-piperidin-1-yl-propoxy)-phenyl]-propan-1-one (2.98 g, 12.0 mmol) in THF (60 mL) was added allylmagnesium bromide (2.0 M in THF; 12 mL, 24 mmol). After 18 h at rt, the mixture was diluted with satd. aq. NaCl and satd. aq. NaHCO₃, and was extracted with EtOAc. The combined organic layers were dried, concentrated, and purified by FCC to give the desired product (2.36 g, 68%). MS (ESI): mass calcd. for C₁₈H₂₇NO₂, 289.20; m/z found, 290.1 [M+H]⁺.

Step B: 1-{3-[4-(1-Phenoxy-but-3-enyl)-phenoxy]-propyl}-piperidine. The title compounds was prepared in an analogous fashion to Example 1, Step E. MS (ESI): mass calcd. for C₂₄H₃₁NO₂, 365.24; m/z found, 366.2 [M+H]⁺.

Step C: 3-Phenoxy-3-[4-(3-piperidin-1-yl-propoxy)-phenyl]-propan-1-ol. Ozone was bubbled through a solution of 1-{3-[4-(1-phenoxy-but-3-enyl)phenoxy]-propyl}-piperidine (445 mg, 0.93 mmol) in CH₂Cl₂ (9 mL) and MeOH (3 mL) at −78° C. until a blue color change was noticed (10 min). The mixture was then placed under a N₂ atmosphere and MeOH (50 mL) and NaBH₄ (1.05 g, 27.9 mmol) were added. After stirring for 2 h at rt, the mixture was diluted with water and satd. aq. NaHCO₃ and extracted with EtOAc (3×). The combined organic layers were dried and concentrated. Purification by FCC gave the desired product (241 mg, 70%). MS (ESI): mass calcd. for C₂₃H₃₁NO₃, 369.23; m/z found, 370.2 [M+H]⁺.

Step D. 2-{3-Phenoxy-3-[4-(3-piperidin-1-yl-propoxy)-phenyl]-propyl}-isoindole-1,3-dione. The title compound was prepared in an analogous fashion to Example 1, Step E. MS (ESI): mass calcd. for C₃₁H₃₄N₂O₄, 498.25; m/z found, 499.3 [M+H]⁺. ¹H NMR (CDCl₃): 7.80 (dd, J=3.0, 5.4, 2H), 7.68 (dd, J=3.0, 5.5, 2H), 7.26 (d, J=8.7, 2H), 7.13 (dd, J=8.0, 8.0, 2H), 6.84-6.75 (m, 5H), 5.17 (dd, J=8.4, 4.4, 1H), 3.99-3.90 (m, 3H), 3.87-3.80 (m, 1H), 2.46-2.33 (m, 7H), 2.21-2.13 (m, 1H), 1.97-1.78 (m, 2H), 1.61-1.55 (m, 4H), 1.47-1.38 (m, 2H).

Step E. To a solution of 2-{3-phenoxy-3-[4-(3-piperidin-1-yl-propoxy)phenyl]-propyl}-isoindole-1,3-dione (68 mg, 0.14 mmol) in CH₂Cl₂/MeOH (3:1, 14 mL) was added hydrazine hydrate (0.100 mL, 2.03 mmol). The mixture was stirred in a stoppered flask at rt for 18 h. An additional aliquot of hydrazine hydrate (0.200 mL, 4.06 mmol) was added and the mixture was heated at reflux for 2 h. The mixture was concentrated and the residue taken up in EtOAc and extracted with 1 M HCl. The aqueous layer was washed with EtOAc (2×) and diluted with satd. aq. NaHCO₃ until basic. The aqueous layer was extracted with EtOAc (3×) and the combined organic layers dried, concentrated, and purified by reverse phase HPLC to give the desired product (30 mg, 60%). MS (ESI): mass calcd. for C₂₃H₃₂N₂O₂, 368.25; m/z found, 369.4 [M+H]⁺. ¹H NMR (acetone-d₆): 7.28-7.24 (m, 2H), 7.09-7.02 (m, 2H), 6.81-6.76 (m, 4H), 6.76-6.71 (m, 1H), 5.39 (dd, J=8.7, 4.5, 1H), 3.96 (t, J=6.0, 2H), 3.95-3.80 (m, 2H), 3.52 (d, J=11.9, 2H), 3.22-3.14 (m, 2H), 2.86 (dt, J=11.8, 3.6, 1H), 2.45-2.22 (m, 3H), 2.20-2.12 (m, 2H), 1.97-1.94 (m, 2H), 1.85-1.73 (m, 4H), 1.71-1.65 (m, 1H), 1.44-1.33 (m, 1H).

### Example 69

### 3-[4-(3-Piperidin-1-yl-propoxy)-phenyl]-3-(4-trifluoromethylphenoxy)-propylamine

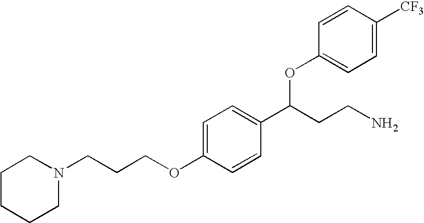

The title compound was prepared in an analogous fashion to Example 68. MS (ESI): mass calcd. for C₂₄H₃₁F₃N₂O₂, 436.23; m/z found, 437.5 [M+H]⁺. ¹H NMR (CDCl₃): 7.41 (d, J=8.7, 2H), 7.23 (d, J=8.6, 2H), 6.93-6.82 (m, 4H), 5.28-5.22 (m, 1H), 3.97 (t, J=6.4, 2H), 2.86 (t, J=6.8, 2H), 2.48-2.30 (m, 6H), 2.20-2.10 (m, 1H), 1.99-1.88 (m, 3H), 1.62-1.53 (m, 4H), 1.52-1.37 (m, 4H). The free base was converted to the maleic acid salt for biological testing.

### Example 70

### Methyl-[3-[4-(3-piperidin-1-yl-propoxy)-phenyl]-3-(4-trifluoromethyl-phenoxy)-propyl]-amine maleic acid salt

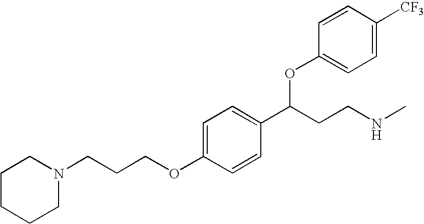

Step A: 3-[4-(3-Piperidin-1-yl-propoxy)-phenyl]-3-(4-trifluoromethylphenoxy)-propan-1-ol. The title compound was prepared in a similar manner as Example 68, Steps A-C.

Step B. Methanesulfonic acid 3-[4-(3-piperidin-1-yl-propoxy)-phenyl]-3-(4-trifluoromethyl-phenoxy)-propyl ester. To a solution of 3-[4-(3-piperidin-1-yl-propoxy)-phenyl]-3-(4-trifluoromethyl-phenoxy)-propan-1-ol (178 mg, 0.482 mmol) and Et₃N (0.445 mL, 3.19 mmol) in THF (3.2 mL) was added MsCl (92.2 mg, 0.638 mmol) in THF (0.64 mL) dropwise. After 18 h at rt, the mixture was diluted with satd. aq. NaHCO₃ and extracted with Et₂O (2×) and CH₂Cl₂ (2×). The combined organic layers were dried and concentrated to give the desired product (212 mg, 98%). MS (ESI): mass calcd. for C₂₅H₃₂F₃NO₅S, 515.20; m/z found, 516.5 [M+H]⁺.

Step C. A solution of methanesulfonic acid 3-[4-(3-piperidin-1-yl-propoxy)-phenyl]-3-(4-trifluoromethyl-phenoxy)-propyl ester (106 mg, 0.237 mmol) and 40% aq. MeNH₂ (5 mL) in THF (5 mL) was heated in a sealed tube at 70° C. for 3 h. The mixture was diluted with satd. aq. NaHCO₃ and extracted with CH₂Cl₂ (3×). The combined organic layers were dried and concentrated. Purification by FCC gave the desired product (55.5 mg, 61%). MS (ESI): mass calcd. for C₂₅H₃₃F₃N₂O₂, 450.25; m/z found, 451.5 [M+H]⁺. ¹H NMR (CDCl₃): 7.41 (d, J=8.6, 2H), 7.23 (d, J=8.7, 2H), 6.89 (d, J=8.6, 2H), 6.85 (d, J=8.7, 2H), 5.27-5.22 (m, 1H), 3.97 (t, J=6.4, 2H), 2.71 (t, J=6.7, 2H), 2.48-2.34 (m, 9H), 2.23-2.13 (m, 1H), 2.02-1.90 (m, 3H), 1.81-1.63 (bs, 1H), 1.62-1.54 (m, 4H), 1.47-1.38 (m, 2H).

Examples 71-73 were prepared in an analogous fashion to Example 70.

### Example 71

### {3-(3,4-Dichloro-phenoxy)-3-[4-(3-piperidin-1-yl-propoxy)-phenyl]-propyl}-methyl-amine

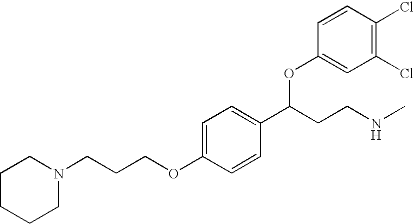

MS (ESI): mass calcd. for C₂₄H₃₂Cl₂N₂O₂, 450.18; m/z found, 451.4 [M+H]⁺. ¹H NMR (CDCl₃): 7.23-7.18 (m, 3H), 6.94 (d, J=2.8, 1H), 8.84 (d, J=8.7, 2H), 6.66 (dd, J=8.9, 2.9, 1H), 5.15-5.11 (m, 1H), 3.97 (t, J=6.4, 2H), 2.69 (t, J=6.7, 2H), 2.47-2.34 (m, 9H), 2.20-2.10 (m, 1H), 2.00-1.90 (m, 4H), 1.62-1.53 (m, 4H), 1.46-1.38 (m, 2H). The free base was converted to the maleic acid salt for biological testing.

### Example 72

### Methyl-{3-phenoxy-3-[4-(3-piperidin-1-yl-propoxy)-phenyl]-propyl}-amine

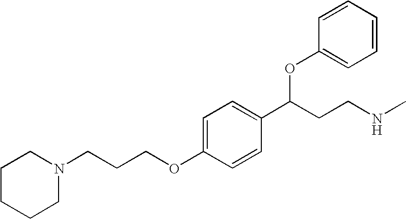

MS (ESI): mass calcd. for C₂₄H₃₄N₂O₂, 382.26; m/z found, 383.3 [M+H]⁺. ¹H NMR (CDCl₃): 7.30-7.21 (m, 2H), 7.21-7.11 (m, 2H), 6.88-6.80 (m, 5H), 5.17 (dd, J=8.1, 4.9, 1H), 3.96 (t, J=6.4, 2H), 2.72 (t, J=6.9, 2H), 2.47-2.42 (m, 2H), 2.41 (s, 3H), 2.40-2.33 (m, 3H), 2.16 (dt, J=14.4, 6.8 Hz, 1H), 2.01 (s, 3H), 1.99-1.90 (m, 3H), 1.79-1.64 (m, 2H), 1.58 (td, J=11.0, 5.6, 1H), 1.47-1.39 (m, 2H). The free base was converted to the maleic acid salt for biological testing.

### Example 73

### 1-{3-[4-(1-Phenoxy-3-pyrrolidin-1-yl-propyl)-phenoxy]-propyl}-piperidine

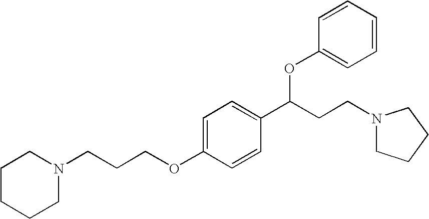

MS (ESI): mass calcd. for C₂₇H₃₈N₂O₂, 422.29; m/z found, 423.4 [M+H]⁺. ¹H NMR (CDCl₃): 7.28-7.23 (m, 2H), 7.19-7.14 (m, 2H), 6.88-6.82 (m, 5H), 5.18-5.13 (m, 1H), 3.97 (t, J=6.4, 2H), 2.64-2.32 (m, 11H), 2.27-2.06 (m, 2H), 2.04-1.90 (m, 3H), 1.80-1.72 (m, 4H), 1.62-1.54 (m, 4H), 1.47-1.39 (m, 2H).

### Example 74

### (−){3-(3,4-Dichloro-phenoxy)-3-[4-(3-piperidin-1-yl-propoxy)phenyl]-propyl}-dimethyl-amine

### Example 75

### (+)-{3-(3,4-Dichloro-phenoxy)-3-[4-(3-piperidin-1-yl-propoxy)phenyl]-propyl}-dimethyl-amine

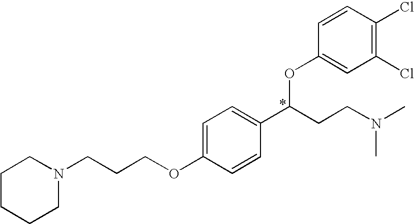

{3-(3,4-Dichloro-phenoxy)-3-[4-(3-piperidin-1-yl-propoxy)-phenyl]-propyl}-dimethyl-amine (Example 9) was separated into enantiomers by chiral chromatography using a 20×250 mm Chiralpak AD column and eluting with 65% EtOH/hexanes with 0.2% diethylamine at a flow rate of 5 mL/min. Example 74 was isolated as the faster (1st) eluting peak with an optical rotation of −44.8. Example 75 was isolated as the slower (2^{nd}) eluting peak with an optical rotation of +33.5.

The compounds in Examples 76-78 were prepared using methods analogous to those described in the preceding examples. Satisfactory analytical data were obtained for each compound.

### Example 76

### Dimethyl-[3-[4-(4-thiomorpholin-4-yl-but-1-ynyl)-phenoxy]-3-(4-trifluoromethyl-phenyl)-propyl]-amine

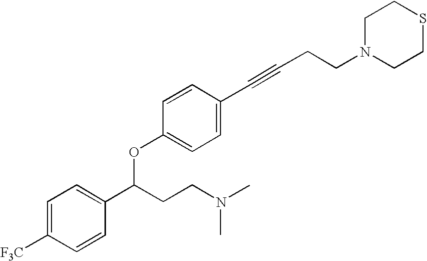

MS (ESI): mass calcd. for C₂₆H₃₁F₃N₂OS, 476.2; m/z found, 477.2 [M+H]⁺.

### Example 77

### {3-(4-Fluoro-phenyl)-3-[4-(4-piperidin-1-yl-but-1-ynyl)-phenoxy]-propyl}-dimethyl-amine

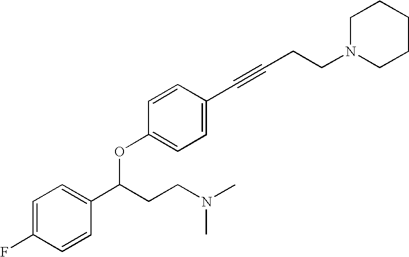

MS (ESI): mass calcd. for C₂₆H₃₃FN₂O, 408.3; m/z found, 409.2 [M+H]⁺.

### Example 78

### (4-Cyclohexyl-piperazin-1-yl)-{4-[3-dimethylamino-1-(4-trifluoromethyl-phenoxy)-propyl]-phenyl}-methanone

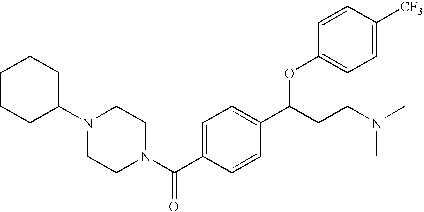

MS (ESI): mass calcd. for C₂₉H₃₈F₃N₂O₂, 517.3; m/z found, 518.6 [M+H]⁺.

The compounds in Examples 79-84 may be prepared according to the methods described in the preceding examples.

### Example 79

### [3-[4-(4-Cyclohexyl-piperazin-1-ylmethyl)-phenyl]-3-(4-trifluoromethyl-phenoxy)-propyl]-dimethyl-amine

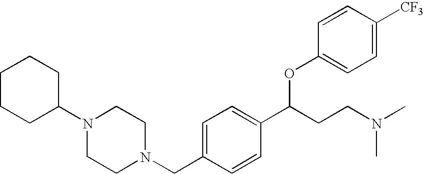

### Example 80

### (3-{4-[3-(4-Fluoro-piperidin-1-yl)-propoxy]-phenyl}-3-phenoxypropyl)-dimethyl-amine

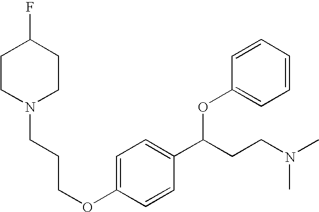

### Example 81

### 1-{3-[4-(3-Dimethylamino-1-phenoxy-propyl)-phenoxy]-propyl}-piperidine-4-carbonitrile

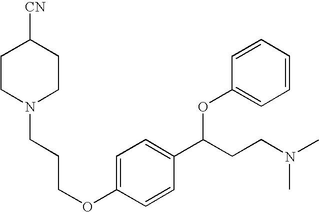

### Example 82

### Dimethyl-(3-{4-[3-(2-methyl-morpholin-4-yl)-propoxy]-phenyl}-3-phenoxy-propyl)-amine

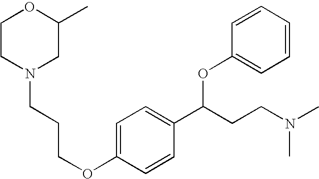

### Example 83

### (4-Cyclopropyl-[1,4]diazepan-1-yl)-{4-[3-dimethylamino-1-(4-trifluoromethyl-phenoxy)-propyl]-phenyl}-methanone

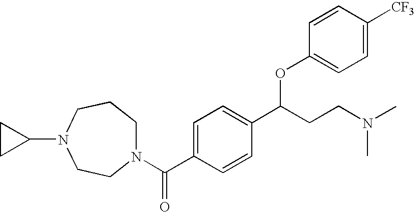

### Example 84

### 1-{3-[4-(3-Azetidin-1-yl-1-phenoxy-propyl)-phenoxy]-propyl}-piperidine

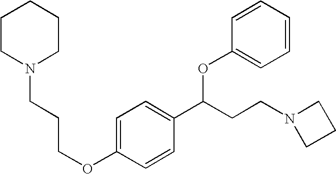

### Biological Methods:

### H₃ Receptor Binding

Binding of compounds to the cloned human H₃ receptor, stably expressed in SK-N-MC cells, was performed (Lovenberg, T. W. et al. J. Pharmacol. Exp. Ther. 2000, 293, 771-778). Briefly, cell pellets from SK-N-MC cells expressing the human H₃ receptor were homogenized in 50 mM Tris-HCl/5 mM EDTA and re-centrifuged at 30,000 g for 30 min. Pellets were re-homogenized in 50 mM Tris/5 mM EDTA (pH 7.4). Membranes were incubated with 0.8 nM N—[³H]-α-methylhistamine plus/minus test compounds for 60 min at 25° C. and harvested by rapid filtration over GF/C glass fiber filters (pretreated with 0.3% polyethylenimine) followed by four washes with ice-cold buffer. Nonspecific binding was defined in the presence of 10 μM histamine. IC₅₀ values were determined by a single site curve-fitting program (GraphPad, San Diego, Calif.) and converted to Kᵢ values based on a N—[³H]-α-methylhistamine K_{d} of 800 μM and a ligand concentration of 800 μM (Cheng & Prusoff, Biochem. Pharmacol. 1973, 22, 3099-3108). Data for compounds tested in this assay are presented in Table 1.

### Rat Brain SERT

A rat brain without cerebellum (Zivic Laboratories, Inc.—Pittsburgh, Pa.) was homogenized in a 52.6 mM Tris pH 8/126.4 mM NaCl/5.26 mM KCl mixture and centrifuged at 1,000 rpm for 5 min. The supernatant was removed and re-centrifuged at 15,000 rpm for 30 min. Pellets were re-homogenized in a 52.6 mM Tris pH8/126.4 mM NaCl/5.26 mM KCl mixture. Membranes were incubated with 0.6 nM [³H]-Citalopram plus/minus test compounds for 60 min at 25° C. and harvested by rapid filtration over GF/C glass fiber filters (pretreated with 0.3% polyethylenimine) followed by four washes with ice-cold buffer. Nonspecific binding was defined in the presence of 100 μM fluoxetine. IC₅₀ values were determined by a single site curve-fitting program (GraphPad, San Diego, Calif.) and converted to Kᵢ values based on a [³H]-Citalopram K_{d} of 0.6 nM and a ligand concentration of 0.6 nM. Data for compounds tested in this assay are presented in Table 1.

TABLE 1

| | Rat SERT | Human H₃ |
|---|---|---|
| Ex. | Kᵢ (nM) | Kᵢ (nM) |
| 1 | 1.0 | 0.7 |
| 2 | 17 | 2.5 |
| 3 | 16 | 3 |
| 4 | 1.3 | 0.9 |
| 5 | 8 | 0.2 |
| 6 | 1.5 | 0.6 |
| 7 | 2 | 0.9 |
| 8 | 2 | 1.7 |
| 9 | 6 | 5 |
| 10 | 1.3 | 1.0 |
| 11 | 5.0 | 1.7 |
| 12 | 4 | 0.9 |
| 13 | 3 | 0.7 |
| 14 | 5 | 1.7 |
| 15 | 2 | 1.0 |
| 16 | 7 | 0.9 |
| 17 | 8 | 0.9 |
| 18 | 9 | 0.3 |
| 19 | 44 | 1.0 |
| 20 | 58 | 9 |
| 21 | 5 | 1.2 |
| 22 | 6 | 49 |
| 23 | 13 | 19 |
| 24 | 18 | 11 |
| 25 | 39 | 21 |
| 26 | 43 | 11 |
| 27 | 80 | 60 |
| 28 | 170 | 122 |
| 29 | 39 | 255 |
| 30 | 21 | 24 |
| 31 | 16 | 2 |
| 32 | 7 | 6 |
| 33 | 3 | 23 |
| 34 | 7 | 12 |
| 35 | 10 | 32 |
| 36 | 36 | 0.8 |
| 37 | 65 | 0.7 |
| 38 | 161 | 0.7 |
| 39 | 42 | 2 |
| 40 | 18 | 2 |
| 41 | 18 | 1.0 |
| 42 | 9 | 2 |
| 43 | 23 | 1.0 |
| 44 | 18 | 0.9 |
| 45 | 65 | 2 |
| 46 | 58 | 1.0 |
| 47 | 6 | 5 |
| 48 | 86 | 17 |
| 49 | 48 | 73 |
| 50 | 53 | 3 |
| 51 | 30 | 7 |
| 52 | 33 | 2 |
| 53 | 102 | 8 |
| 54 | 37 | 5 |
| 55 | 27 | 2 |
| 56 | 14 | 1.0 |
| 57 | 22 | 2 |
| 58 | 150 | 29 |
| 59 | 181 | 56 |
| 60 | 168 | 24 |
| 61 | 108 | 102 |
| 62 | 166 | 143 |
| 63 | 52 | 18 |
| 64 | 342 | 4000 |
| 65 | 1.0 | 0.8 |
| 66 | 15 | 3.0 |
| 67 | 4 | 0.6 |
| 68 | 7 | 0.8 |
| 69 | 5 | 8 |
| 70 | 3 | 1.0 |
| 71 | 3 | 2 |
| 72 | 5 | 0.9 |
| 73 | 633 | 0.2 |
| 74 | 11 | 4 |
| 75 | 9.7 | 4.3 |
| 76 | 537 | 75 |
| 77 | 165 | 364 |
| 78 | 223 | 22 |

### C. Human SERT

Homogenized HEK293 (Human Embryonic Kidney) membranes expressing the human SERT were incubated with ³H-citalopram (SERT) at rt for 1 h in 50 mM Tris, 120 mM NaCl, 5 mM KCl (pH 7.4). Nonspecific binding was determined in the presence of 10 μM fluoxetine for the SERT. The membranes were washed and the radioactivity was counted as above. Calculations for Kᵢ at the SERT were based on a K_{d} value for ³H-citalopram and a ligand concentration of 3.1 nM. Data for compounds tested are presented in Table 2.

TABLE 2

| | Human SERT |
|---|---|
| Ex. | Kᵢ (nM) |
| 1 | 26 |
| 2 | 14 |
| 3 | 199 |
| 4 | 10 |
| 5 | 158 |
| 6 | 16 |
| 7 | 60 |
| 8 | 16 |
| 9 | 5 |
| 10 | 46 |
| 11 | 7 |
| 12 | 9 |
| 13 | 11 |
| 14 | 7 |
| 15 | 4 |
| 16 | 10 |
| 17 | 21 |
| 18 | 91 |
| 20 | 61 |
| 21 | 29 |
| 22 | 22 |
| 23 | 37 |
| 24 | 34 |
| 31 | 154 |
| 32 | 10 |
| 33 | 5 |
| 34 | 10 |
| 35 | 6 |
| 36 | 169 |
| 37 | 1333 |
| 38 | 245 |
| 39 | 42 |
| 40 | 7 |
| 41 | 282 |
| 42 | 180 |
| 43 | 163 |
| 44 | 90 |
| 47 | 6 |
| 56 | 133 |
| 57 | 172 |
| 65 | 14 |
| 66 | 151 |
| 67 | 53 |
| 68 | 21 |
| 69 | 1 |
| 70 | 2 |
| 71 | 0.8 |
| 72 | 25 |
| 73 | 4000 |
| 74 | 3 |
| 75 | 7.3 |

### D. Cyclic AMP Accumulation

Sublines of SK-N-MC cells were created that expressed a reporter construct and the human H₃ receptor. The reporter gene (β-galactosidase) is under the control of multiple cyclic AMP responsive elements. In 96-well plates, histamine was added directly to the cell media followed 5 min later by an addition of forskolin (5 μM final concentration). When appropriate, antagonists were added 10 min prior to agonist addition. After a 6-h incubation at 37° C., the media was aspirated and the cells washed with 200 μL of phosphate-buffered saline followed by a second aspiration. Cells were lysed with 25 μL 0.1× assay buffer (10 mM Na-phosphate, pH 8, 0.2 mM MgSO₄, 0.01 mM MnCl₂) and incubated at rt for 10 min. Cells were then incubated for 10 min with 100 μL of 1× assay buffer containing 0.5% Triton and 40 mM β-mercaptoethanol. Color was developed using 25 μL of 1 mg/mL substrate solution (chlorophenolred β-D galactopyranoside; Roche Molecular Biochemicals, Indianapolis, Ind.). Color was quantitated on a microplate reader at absorbance 570 nM. The pA₂ values were calculated by Schild regression analysis of the pEC₅₀ values. Data for compounds tested are presented in Table 3.

TABLE 3

| Ex. | pA₂ |
|---|---|
| 1 | 9.33 |
| 2 | 8.53 |
| 9 | 8.15 |
| 13 | 9.21 |
| 15 | 8.80 |
| 18 | 10.2 |
| 19 | 9.01 |
| 20 | 8.25 |
| 21 | 9.18 |
| 32 | 8.32 |
| 36 | 8.16 |
| 38 | 9.67 |
| 39 | 9.03 |
| 40 | 8.66 |
| 41 | 9.12 |
| 42 | 8.41 |
| 43 | 8.77 |
| 44 | 10.01 |
| 45 | 8.83 |
| 45 | 8.83 |
| 47 | 7.94 |
| 50 | 8.83 |
| 51 | 7.95 |
| 52 | 8.92 |
| 53 | 8.61 |
| 54 | 7.73 |
| 55 | 8.03 |
| 56 | 9.01 |
| 57 | 8.66 |
| 60 | 7.75 |
| 65 | 9.60 |
| 66 | 8.40 |
| 67 | 9.20 |
| 69 | 8.10 |
| 70 | 8.40 |
| 71 | 7.98 |
| 74 | 8.23 |
| 75 | 8.15 |

What is claimed is:

1. A compound of Formula (I): 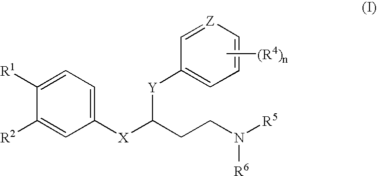
wherein:
one of X and Y is O, S, NH, or CH₂, and the other is a bond;
Z is CH or N, with the proviso that Z is N only when Y is O;
one of R¹ and R² is -Q and the other is —H;
-Q is —OCH(R^{a})(CH₂)₂NR^{b}R^{c}, —CZ-C(CH₂)₂NR^{b}R^{c}, —(CH₂)₄NR^{b}R^{c}, —CH₂NR^{b}R^{c}, or —C(O)NR^{b}R^{c};
wherein R^{a} is —H or is taken together with R^{b} to form ethylene; and
R^{b} and R^{c} are —H or —C₁₋₆alkyl, or R^{b} and R^{c} taken together with their nitrogen of attachment form a heterocycloalkyl ring, unsubstituted or substituted with C₁₋₆alkyl, C₃₋₆cycloalkyl, fluoro, or —CN;
each R⁴ substituent is independently selected from the group consisting of halo, —C₁₋₆alkyl, —CHF₂, —CF₃, —OH, —OC₁₋₆alkyl, —OCHF₂, —OCF₃, —CN, —N(R^{k})R^{l}, —NO₂, —SC₁₋₆alkyl, —SCF₃, and —S(O)₀₋₂—C₁₋₆alkyl; or, alternatively, only when Y is O, two adjacent R⁴ substituents taken together with the phenyl to which they are attached form indol-5-yl;
wherein R^{k} and R^{l} are each independently —H or —C₁₋₆alkyl;
n is 0, 1, 2, or 3;
R⁵ is —H or —C₁₋₄alkyl; and R⁶ is —C₁₋₄alkyl, or, alternatively, R⁵ and R⁶ taken together with their nitrogen of attachment form a heterocycloalkyl ring;
or a pharmaceutically acceptable salt, pharmaceutically acceptable prodrug, or pharmaceutically active metabolite of such compound.

2. A compound as defined in claim 1, wherein X is O, S, NH, or CH₂ and Y is a bond.

3. A compound as defined in claim 1, wherein Y is O, S, NH, or CH₂ and X is a bond.

4. A compound as defined in claim 1, wherein X is a bond and Y is O.

5. A compound as defined in claim 1, wherein Z is CH.

6. A compound as defined in claim 1, wherein -Q is —O(CH₂)₃NR^{b}R^{c}.

7. A compound as defined in claim 1, wherein -Q is —CZ-C(CH₂)₂NR^{b}R^{c} or —(CH₂)₄NR^{b}R^{c}.

8. A compound as defined in claim 1, wherein -Q is —CH₂NR^{b}R^{c} or —C(O)NR^{b}R^{c}.

9. A compound as defined in claim 1, wherein R¹ is -Q, R² is —H, and -Q is —O(CH₂)₃NR^{b}R^{c}.

10. A compound as defined in claim 1, wherein R^{b} and R^{c} are each independently —H, methyl, ethyl, propyl, isopropyl, butyl, isobutyl, tert-butyl, pentyl, or hexyl.

11. A compound as defined in claim 1, wherein R^{b} and R^{c} taken together with their nitrogen of attachment form a 5- to 7-membered heterocycloalkyl ring, unsubstituted or substituted with methyl, ethyl, isopropyl, butyl, isobutyl, sec-butyl, isopentyl, cyclopropyl, cyclobutyl, cyclopentyl, cyclohexyl, fluoro, or cyano.

12. A compound as defined in claim 1, wherein R^{b} and R^{c} taken together with their nitrogen of attachment form diazepine, piperidine, piperazine, morpholine, or thiomorpholine, each unsubstituted or substituted with methyl, ethyl, isopropyl, butyl, isobutyl, sec-butyl, isopentyl, cyclopropyl, cyclobutyl, cyclopentyl, cyclohexyl, fluoro, or cyano.

13. A compound as defined in claim 1, wherein R^{b} and R^{c} taken together with their nitrogen of attachment form piperidin-1-yl, 4-fluoro-piperidin-1-yl, 4-cyano-piperidin-1-yl, 4-isopropyl-piperazin-1-yl, morpholinyl, 3-methyl-morpholin-1-yl, thiomorpholinyl, 4-butyl-piperazin-1-yl, 4-cyclopropyl-piperazin-1-yl, 4-sec-butyl-piperazin-1-yl, 4-(1-ethyl-propyl)-piperazin-1-yl, 4-cyclopentyl-piperazin-1-yl, or 4-cyclohexyl-piperazin-1-yl.

14. A compound as defined in claim 1, wherein each R⁴ substituent is independently selected from the group consisting of chloro, bromo, methyl, —CF₃, methoxy, —NO₂, and methanesulfanyl.

15. A compound as defined in claim 1, wherein n is 1 or 2.

16. A compound as defined in claim 1, wherein R⁵ and R⁶ are both methyl.

17. A compound as defined in claim 1, wherein R⁵ and R⁶ taken together with their nitrogen of attachment form azetidinyl, pyrrolidinyl, or piperidinyl.

18. A compound of Formula (II): 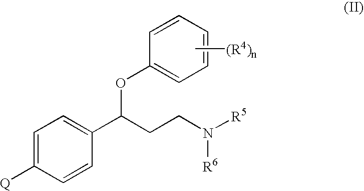
wherein:
-Q is —OCH(R^{a})(CH₂)₂NR^{b}R^{c}, —C≡C(CH₂)₂NR^{b}R^{c}, —(CH₂)₄NR^{b}R^{c}, —CH₂NR^{b}R^{c}, or —C(O)NR^{b}R^{c};
wherein R^{a} is —H or is taken together with R^{b} to form ethylene; and
R^{b} and R^{c} are —H or —C₁₋₆alkyl, or R^{b} and R^{c} taken together with their nitrogen of attachment form a heterocycloalkyl ring, unsubstituted or substituted with C₁₋₆alkyl, C₃₋₆cycloalkyl, fluoro, or —CN;
each R⁴ substituent is independently selected from the group consisting of halo, —C₁₋₆alkyl, —CHF₂, —CF₃, —OH, —OC₁₋₆alkyl, —OCHF₂, —OCF₃, —CN, —N(R^{k})R^{l}, —NO₂, —SC₁₋₆alkyl, —SCF₃, and —S(O)₀₋₂—C₁₋₆alkyl; or, alternatively, two adjacent R⁴ substituents taken together with the phenyl to which they are attached form indol-5-yl;
wherein R^{k} and R^{l} are each independently —H or —C₁₋₆alkyl;
n is 0, 1, 2, or 3;
R⁵ is —H or —C₁₋₄alkyl; and R⁶ is —C₁₋₄alkyl, or, alternatively, R⁵ and R⁶ taken together with their nitrogen of attachment form a heterocycloalkyl ring;
or a pharmaceutically acceptable salt, pharmaceutically acceptable prodrug, or pharmaceutically active metabolite of such compound.

19. A compound according to claim 18, wherein -Q is —OCH(R^{a})(CH₂)₂NR^{b}R^{c}.

20. A compound selected from the group consisting of:
Dimethyl-{3-phenoxy-3-[4-(3-piperidin-1-yl-propoxy)-phenyl]-propyl}-amine;
Dimethyl-[3-[4-(3-piperidin-1-yl-propoxy)-phenyl]-3-(4-trifluoromethyl-phenoxy)propyl]-amine;
Dimethyl-{3-phenoxy-3-[3-(3-piperidin-1-yl-propoxy)-phenyl]-propyl}-amine;
Dimethyl-{3-(4-methylsulfanyl-phenoxy)-3-[4-(3-piperidin-1-yl-propoxy)-phenyl]-propyl}-amine;
4-{3-Dimethylamino-1-[4-(3-piperidin-1-yl-propoxy)-phenyl]-propoxy}-benzonitrile;
Dimethyl-{3-[4-(3-piperidin-1-yl-propoxy)-phenyl]-3-p-tolyloxy-propyl}-amine;
{3-(4-Methoxy-phenoxy)-3-[4-(3-piperidin-1-yl-propoxy)-phenyl]-propyl}-dimethyl-amine;
{3-(4-Chloro-phenoxy)-3-[4-(3-piperidin-1-yl-propoxy)-phenyl]-propyl}-dimethyl-amine;
{3-(3,4-Dichloro-phenoxy)-3-[4-(3-piperidin-1-yl-propoxy)-phenyl]-propyl}-dimethyl-amine;
{3-(4-Fluoro-phenoxy)-3-[4-(3-piperidin-1-yl-propoxy)-phenyl]-propyl}-dimethyl-amine;
{3-(4-Bromo-phenoxy)-3-[4-(3-piperidin-1-yl-propoxy)-phenyl]-propyl}-dimethyl-amine;
Dimethyl-{3-(4-nitro-phenoxy)-3-[4-(3-piperidin-1-yl-propoxy)-phenyl]-propyl}-amine;
{3-(3-Methoxy-phenoxy)-3-[4-(3-piperidin-1-yl-propoxy)-phenyl]-propyl}-dimethyl-amine;
{3-(3-Chloro-phenoxy)-3-[4-(3-piperidin-1-yl-propoxy)-phenyl]-propyl}-dimethyl-amine;
{3-(3-Bromo-phenoxy)-3-[4-(3-piperidin-1-yl-propoxy)-phenyl]-propyl}-dimethyl-amine;
{3-(2,4-Dichloro-phenoxy)-3-[4-(3-piperidin-1-yl-propoxy)-phenyl]-propyl}-dimethyl-amine;
{3-(2-Chloro-phenoxy)-3-[4-(3-piperidin-1-yl-propoxy)-phenyl]-propyl}-dimethyl-amine;
Dimethyl-[3-[4-(3-piperidin-1-yl-propoxy)-phenyl]-3-(pyridin-3-yloxy)-propyl]-amine;
{3-(1H-Indol-5-yloxy)-3-[4-(3-piperidin-1-yl-propoxy)-phenyl]-propyl}-dimethyl-amine;
Dimethyl-[3-[4-(4-piperidin-1-yl-but-1-ynyl)-phenyl]-3-(4-trifluoromethylphenoxy)-propyl]-amine;
Dimethyl-{3-phenoxy-3-[4-(4-piperidin-1-yl-but-1-ynyl)-phenyl]-propyl}-amine;
(3-{4-[4-(4-Isopropyl-piperazin-1-yl)-but-1-ynyl]-phenyl}-3-phenoxy-propyl)dimethyl-amine;
Dimethyl-{3-[4-(4-morpholin-4-yl-but-1-ynyl)-phenyl]-3-phenoxy-propyl}-amine;
Dimethyl-{3-phenoxy-3-[4-(4-thiomorpholin-4-yl-but-1-ynyl)-phenyl]-propyl}-amine;
{3-(4-Chloro-phenoxy)-3-[4-(4-piperidin-1-yl-but-1-ynyl)-phenyl]-propyl}-dimethyl-amine;
{3-(4-Methoxy-phenoxy)-3-[4-(4-thiomorpholin-4-yl-but-1-ynyl)-phenyl]-propyl}-dimethyl-amine;
Dimethyl-[3-[4-(4-morpholin-4-yl-but-1-ynyl)-phenyl]-3-(4-trifluoromethylphenoxy)-propyl]-amine;
Dimethyl-[3-[4-(4-thiomorpholin-4-yl-but-1-ynyl)-phenyl]-3-(4-trifluoromethylphenoxy)-propyl]-amine;
[3-{4-[4-(4-Isopropyl-piperazin-1-yl)-but-1-ynyl]-phenyl}-3-(4-trifluoromethylphenoxy)-propyl]-dimethyl-amine;
{3-(3,4-Dichloro-phenoxy)-3-[4-(4-piperidin-1-yl-but-1-ynyl)-phenyl]-propyl}-dimethyl-amine;
Dimethyl-{3-phenoxy-3-[3-(4-piperidin-1-yl-but-1-ynyl)-phenyl]-propyl}-amine;
Dimethyl-{3-phenoxy-3-[4-(4-piperidin-1-yl-butyl)-phenyl]-propyl}-amine;
Dimethyl-[3-[4-(4-piperidin-1-yl-butyl)-phenyl]-3-(4-trifluoromethyl-phenoxy)propyl]-amine;
[3-{4-[4-(4-Isopropyl-piperazin-1-yl)-butyl]-phenyl}-3-(4-trifluoromethylphenoxy)-propyl]-dimethyl-amine;
Dimethyl-[3-[4-(4-morpholin-4-yl-butyl)-phenyl]-3-(4-trifluoromethyl-phenoxy)propyl]-amine;
{3-[4-(1-Isopropyl-piperidin-4-yloxy)-phenyl]-3-phenoxy-propyl}-dimethyl-amine;
Dimethyl-{3-phenyl-3-[3-(3-piperidin-1-yl-propoxy)-phenoxy]-propyl}-amine;
[4-(3-Dimethylamino-1-phenoxy-propyl)-phenyl]-(4-isopropyl-piperazin-1-yl)methanone;
{4-[3-Dimethylamino-1-(4-trifluoromethyl-phenoxy)-propyl]-phenyl}-(4-isopropyl-piperazin-1-yl)-methanone;
{4-[1-(3,4-Dichloro-phenoxy)-3-dimethylamino-propyl]-phenyl}-(4-isopropyl-piperazin-1-yl)-methanone;
Dimethyl-{3-phenyl-3-[4-(3-piperidin-1-yl-propoxy)-phenoxy]-propyl}-amine;
Dimethyl-[3-[4-(3-piperidin-1-yl-propoxy)-phenoxy]-3-(4-trifluoromethyl-phenyl)propyl]-amine;
{3-(4-Chloro-phenyl)-3-[4-(3-piperidin-1-yl-propoxy)-phenoxy]-propyl}-dimethyl-amine;
{3-[4-(1-Isopropyl-piperidin-4-yloxy)-phenoxy]-3-phenyl-propyl}-dimethyl-amine;
[3-[4-(1-Isopropyl-piperidin-4-yloxy)-phenoxy]-3-(4-trifluoromethyl-phenyl)propyl]-dimethyl-amine;
{4-[3-Dimethylamino-1-(4-trifluoromethyl-phenyl)-propoxy]-phenyl}-(4-isopropyl-piperazin-1-yl)-methanone;
{3-(3,4-Dichloro-phenoxy)-3-[4-(4-isopropyl-piperazin-1-ylmethyl)-phenyl]-propyl}-dimethyl-amine;
(4-Butyl-piperazin-1-yl)-{4-[3-dimethylamino-1-(4-trifluoromethyl-phenoxy)propyl]-phenyl}-methanone;
[3-[4-(4-Butyl-piperazin-1-ylmethyl)-phenyl]-3-(4-trifluoromethyl-phenoxy)propyl]-dimethyl-amine;
(4-Cyclopentyl-piperazin-1-yl)-{4-[3-dimethylamino-1-(4-trifluoromethylphenoxy)-propyl]-phenyl}-methanone;
[3-[4-(4-Cyclopentyl-piperazin-1-ylmethyl)-phenyl]-3-(4-trifluoromethylphenoxy)-propyl]-dimethyl-amine;
(4-sec-Butyl-piperazin-1-yl)-{4-[3-dimethylamino-1-(4-trifluoromethyl-phenoxy)propyl]-phenyl}-methanone;
{4-[3-Dimethylamino-1-(4-trifluoromethyl-phenoxy)-propyl]-phenyl}-[4-(1-ethylpropyl)-piperazin-1-yl]-methanone;
[3-{4-[4-(1-Ethyl-propyl)-piperazin-1-ylmethyl]-phenyl}-3-(4-trifluoromethylphenoxy)-propyl]-dimethyl-amine;
[3-[4-(4-Isopropyl-piperazin-1-ylmethyl)-phenyl]-3-(4-trifluoromethyl-phenoxy)propyl]-dimethyl-amine;
{3-[4-(4-Isopropyl-piperazin-1-ylmethyl)-phenyl]-3-phenoxy-propyl}-dimethyl-amine;
Dimethyl-{3-phenyl-3-[4-(4-piperidin-1-yl-but-1-ynyl)-phenoxy]-propyl}-amine;
{3-(4-Chloro-phenyl)-3-[4-(4-thiomorpholin-4-yl-but-1-ynyl)-phenoxy]-propyl}-dimethyl-amine;
{3-(4-Chloro-phenyl)-3-[4-(4-morpholin-4-yl-but-1-ynyl)-phenoxy]-propyl}-dimethyl-amine;
Dimethyl-[3-[4-(4-piperidin-1-yl-but-1-ynyl)-phenoxy]-3-(4-trifluoromethylphenyl)-propyl]-amine;
Dimethyl-{3-[4-(4-morpholin-4-yl-but-1-ynyl)-phenoxy]-3-phenyl-propyl}-amine;
[3-{4-[4-(4-Isopropyl-piperazin-1-yl)-but-1-ynyl]-phenoxy}-3-(4-trifluoromethylphenyl)-propyl]-dimethyl-amine;
Dimethyl-{3-phenyl-3-[4-(4-thiomorpholin-4-yl-but-1-ynyl)-phenoxy]-propyl}-amine;
Dimethyl-[3-[4-(4-morpholin-4-yl-but-1-ynyl)-phenoxy]-3-(4-trifluoromethylphenyl)-propyl]-amine;
Dimethyl-{4-phenyl-3-[4-(3-piperidin-1-yl-propoxy)-phenyl]-butyl}-amine;
N³,N³-Dimethyl-N¹-phenyl-1-[4-(3-piperidin-1-yl-propoxy)-phenyl]-propane-1,3-diamine;
Dimethyl-{3-phenylsulfanyl-3-[4-(3-piperidin-1-yl-propoxy)-phenyl]-propyl}-amine;
3-Phenoxy-3-[4-(3-piperidin-1-yl-propoxy)-phenyl]-propylamine;
3-[4-(3-Piperidin-1-yl-propoxy)-phenyl]-3-(4-trifluoromethyl-phenoxy)propylamine;
Methyl-[3-[4-(3-piperidin-1-yl-propoxy)-phenyl]-3-(4-trifluoromethyl-phenoxy)propyl]-amine;
{3-(3,4-Dichloro-phenoxy)-3-[4-(3-piperidin-1-yl-propoxy)-phenyl]-propyl}-methyl-amine;
Methyl-{3-phenoxy-3-[4-(3-piperidin-1-yl-propoxy)-phenyl]-propyl}-amine;
1-{3-[4-(1-Phenoxy-3-pyrrolidin-1-yl-propyl)-phenoxy]-propyl}-piperidine;
(−)-{3-(3,4-Dichloro-phenoxy)-3-[4-(3-piperidin-1-yl-propoxy)-phenyl]-propyl}-dimethyl-amine;
(+)-{3-(3,4-Dichloro-phenoxy)-3-[4-(3-piperidin-1-yl-propoxy)-phenyl]-propyl}-dimethyl-amine;
Dimethyl-[3-[4-(4-thiomorpholin-4-yl-but-1-ynyl)-phenoxy]-3-(4-trifluoromethylphenyl)-propyl]-amine;
{3-(4-Fluoro-phenyl)-3-[4-(4-piperidin-1-yl-but-1-ynyl)-phenoxy]-propyl}-dimethyl-amine;
(4-Cyclohexyl-piperazin-1-yl)-{4-[3-dimethylamino-1-(4-trifluoromethylphenoxy)-propyl]-phenyl}-methanone;
{3-[4-(4-Cyclohexyl-piperazin-1-ylmethyl)-phenyl]-3-phenoxy-propyl}-dimethyl-amine;
(3-{4-[3-(4-Fluoro-piperidin-1-yl)-propoxy]-phenyl}-3-phenoxy-propyl)-dimethyl-amine;
1-{3-[4-(3-Dimethylamino-1-phenoxy-propyl)-phenoxy]-propyl}-piperidine-4-carbonitrile;
Dimethyl-(3-{4-[3-(2-methyl-morpholin-4-yl)-propoxy]-phenyl}-3-phenoxypropyl)-amine;
(4-Cyclopropyl-[1,4]diazepan-1-yl)-{4-[3-dimethylamino-1-(4-trifluoromethylphenoxy)-propyl]-phenyl}-methanone; and
1-{3-[4-(3-Azetidin-1-yl-1-phenoxy-propyl)-phenoxy]-propyl}-piperidine;
and pharmaceutically acceptable salts thereof.

21. A pharmaceutical composition for treating a disease, disorder, or medical condition mediated by histamine H₃ receptor and/or serotonin transporter activity, comprising:
(a) an effective amount of a compound of Formula (I): 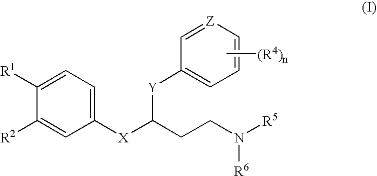
wherein:
one of X and Y is O, S, NH, or CH₂, and the other is a bond;
Z is CH or N, with the proviso that Z is N only when Y is O;
one of R¹ and R² is -Q and the other is —H;
-Q is —OCH(R^{a})(CH₂)₂NR^{b}R^{c}, —C≡C(CH₂)₂NR^{b}R^{c}, —(CH₂)₄NR^{b}R^{c}, —CH₂NR^{b}R^{c}, or —C(O)NR^{b}R^{c};
wherein R^{a} is —H or is taken together with R^{b} to form ethylene; and
R^{b} and R^{c} are —H or —C₁₋₆alkyl, or R^{b} and R^{c} taken together with their nitrogen of attachment form a heterocycloalkyl ring, unsubstituted or substituted with C₁₋₆alkyl, C₃₋₆cycloalkyl, fluoro, or —CN;
each R⁴ substituent is independently selected from the group consisting of halo, —C₁₋₆alkyl, —CHF₂, —CF₃, —OH, —OC₁₋₆alkyl, —OCHF₂, —OCF₃, —CN, —N(R^{k})R^{l}, —NO₂, —SC₁₋₆alkyl, —SCF₃, and —S(O)₀₋₂—C₁₋₆alkyl; or, alternatively, only when Y is O, two adjacent R⁴ substituents taken together with the phenyl to which they are attached form indol-5-yl;
wherein R^{k} and R^{l} are each independently —H or —C₁₋₆alkyl;
n is 0, 1, 2, or 3;
R⁵ is —H or —C₁₋₄alkyl; and R⁶ is —C₁₋₄alkyl, or, alternatively, R⁵ and R⁶ taken together with their nitrogen of attachment form a heterocycloalkyl ring;
or a pharmaceutically acceptable salt, pharmaceutically acceptable prodrug, or pharmaceutically active metabolite thereof; and
(b) a pharmaceutically acceptable excipient.

22. A method of treating a subject suffering from or diagnosed with a disease, disorder, or medical condition mediated by histamine H₃ receptor and/or serotonin transporter activity, comprising administering to the subject in need of such treatment an effective amount of a compound of Formula (I): 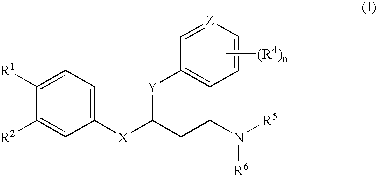
wherein:
one of X and Y is O, S, NH, or CH₂, and the other is a bond;
Z is CH or N, with the proviso that Z is N only when Y is O;
one of R¹ and R² is -Q and the other is —H;
-Q is —OCH(R^{a})(CH₂)₂NR^{b}R^{c}, —C≡C(CH₂)₂NR^{b}R^{c}, —(CH₂)₄NR^{b}R^{c}, —CH₂NR^{b}R^{c}, or —C(O)NR^{b}R^{c};
wherein R^{a} is —H or is taken together with R^{b} to form ethylene; and
R^{b} and R^{c} are —H or —C₁₋₆alkyl, or R^{b} and R^{c} taken together with their nitrogen of attachment form a heterocycloalkyl ring, unsubstituted or substituted with C₁₋₆alkyl, C₃₋₆cycloalkyl, fluoro, or —CN;
each R⁴ substituent is independently selected from the group consisting of halo, —C₁₋₆alkyl, —CHF₂, —CF₃, —OH, —OC₁₋₆alkyl, —OCHF₂, —OCF₃, —CN, —N(R^{k})R^{l}, —NO₂, —SC₁₋₆alkyl, —SCF₃, and —S(O)₀₋₂—C₁₋₆alkyl; or, alternatively, only when Y is O, two adjacent R⁴ substituents taken together with the phenyl to which they are attached form indol-5-yl;
wherein R^{k} and R^{l} are each independently —H or —C₁₋₆alkyl;
n is 0, 1, 2, or 3;
R⁵ is —H or —C₁₋₄alkyl; and R⁶ is —C₁₋₄alkyl, or, alternatively, R⁵ and R⁶ taken together with their nitrogen of attachment form a heterocycloalkyl ring;
or a pharmaceutically acceptable salt, pharmaceutically acceptable prodrug, or pharmaceutically active metabolite thereof.

23. The method according to claim 22, wherein the disease, disorder, or medical condition is selected from the group consisting of: dementia, Alzheimer's disease, cognitive dysfunction, mild cognitive impairment, pre-dementia, attention deficit hyperactivity disorders, attention-deficit disorders, and learning and memory disorders.

24. The method according to claim 22, wherein the disease, disorder, or medical condition is selected from the group consisting of: learning impairment, memory impairment, age-related cognitive decline, memory loss, insomnia, disturbed sleep, narcolepsy with or without associated cataplexy, cataplexy, disorders of sleep/wake homeostasis, idiopathic somnolence, excessive daytime sleepiness, circadian rhythm disorders, fatigue, lethargy, jet lag, REM-behavioral disorder, sleep apnea, perimenopausal hormonal shifts, Parkinson's disease, multiple sclerosis, depression, chemotherapy, shift work schedules, schizophrenia, bipolar disorders, manic disorders, depression, obsessive-compulsive disorder, post-traumatic stress disorder, motion sickness, vertigo, benign postural vertigo, tinitus, epilepsy, migraine, neurogenic inflammation, eating disorders, obesity, substance abuse disorders, sexual dysfunction, premature ejaculation, movement disorders, restless leg syndrome, eye-related disorders, macular degeneration, and retinitis pigmentosis.

25. The method according to claim 22, wherein the disease, disorder, or medical condition is selected from the group consisting of: depression, disturbed sleep, fatigue, lethargy, cognitive impairment, memory impairment, memory loss, learning impairment, attention-deficit disorders, and eating disorders.
